(19) United States
(12) Patent Application Publication
(10) Pub. No.: US 2024/0173311 A1
(43) Pub. Date: May 30, 2024

(54) METHODS AND COMPOSITIONS FOR MODULATING FGF ACTIVITY

(71) Applicant: Recovery Therapeutics, Inc., Needham, MA (US)

(72) Inventors: Seth P. FINKLESTEIN, Needham, MA (US); Gregory D. CUNY, Houston, TX (US); Renato SKERLJ, Newton, MA (US); Soumya RAY, Quincy, MA (US); Stephen Douglas BARRETT, Hartland, MI (US); Kirk Lang OLSON, Canton, MI (US)

(21) Appl. No.: 18/276,943

(22) PCT Filed: Feb. 11, 2022
(86) PCT No.: PCT/US2022/016159

§ 371 Date: Aug. 11, 2023

Related U.S. Application Data
(60) Provisional application No. 63/148,900, filed on Feb. 12, 2021.

(51) Int. Cl. A61K 31/445; A61K 31/136; A61K 31/15; A61K 31/165; A61K 31/192; A61K 31/195; A61K 31/351; A61K 31/382; A61K 31/426; A61P 25/00

(52) U.S. Cl. CPC A61K 31/445; A61K 31/136; A61K 31/15; A61K 31/165; A61K 31/192; A61K 31/195; A61K 31/351; A61K 31/382; A61K 31/426; A61P 25/00

(57) ABSTRACT

The invention features compounds and a method of treating an injury or a disease e.g., stroke, congenital hypogonadotropic hypogonadism, and viral infection, using the compounds. Also featured is a pharmaceutical composition containing one or more of the compounds, and a method of increasing spermatogenesis using the compounds.

### STATEMENT REGARDING FEDERALLY SPONSORED RESEARCH

This invention was made with government support under grant number 2R44 NS095381-02 from the National Institutes of Health. The government has certain rights to the invention.

### BACKGROUND OF THE INVENTION

Stroke is a medical condition caused by a lack of blood supply or bleeding into the brain. Stroke is a leading cause of death in the U.S., and affects approximately 800,000 people per year. Survivors of stroke live an average of seven years after stroke, and approximately 40% of survivors have severe mobility issues. There is a lack of effective treatments for stroke and methods for improving the recovery of stroke survivors.

Several growth factors, such as Fibroblast Growth Factors or FGFs, appear to stimulate the process of stroke recovery. In particular, FGF-2, a member of the FGF polypeptide family, supports the survival and outgrowth of a wide variety of neurons in the brain. Previous experimental studies in animals have shown that endogenous FGF-2 and its receptors, e.g., FGF-R1, are up-regulated after stroke, and exogenously administered FGF-2 can enhance spontaneous recovery after stroke, perhaps through increasing neuronal sprouting and new synapse formation in intact brain tissue surrounding the stroke and on the other side of the brain (Kawamata et al., Proc Natl Acad Sci. 94:8179-84, 1997). An additional mechanism may be stimulation of progenitor cell proliferation, migration, and differentiation in brain (Wada et al., Stroke. 34:2722-2728, 2003). However, FGF-2 is a 155-amino acid polypeptide of approximately 18 kDa, which makes the polypeptide challenging to use as a therapy for stroke and other brain injuries and diseases.

There exists a need for novel therapies to increase FGF-2 signaling activity and to enhance the binding between FGF-2 and its receptors, e.g., FGF-R1. Such compounds and therapies are useful in methods for treatment of stroke and other brain injuries and diseases, such as traumatic brain injury (TBI).

### SUMMARY OF THE INVENTION

The invention provides methods for treating various diseases, injuries, and disorders, e.g., modulated by FGF activity, and effecting other desirable outcomes. In particular, compounds of the invention may be used in the treatment of stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection).

In a first aspect, the invention features a method of treating a subject having a disease or injury comprising administering to the subject a therapeutically effective amount of a compound, wherein the compound is a compound of formula (I):

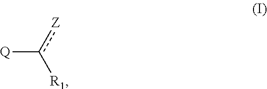

or a pharmaceutically acceptable salt or a tautomer thereof, in which Q is optionally substituted C₆-C₁₀ aryl or optionally substituted 6- to 10-membered heterocyclyl; R₁ is H, OH, optionally substituted C₁-C₆ alkyl, optionally substituted C₆-C₁₆ aryl, or optionally substituted 6- to 12-membered heteroaryl; and Z is O or NR_{c} and [symbol] is a double bond, wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein R_{d} and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, form an optionally substituted 6- to 10-membered heterocyclyl, or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol] is a single bond, and R₁ and Z, together with the carbon atom to which they are attached, form an optionally substituted oxazolidinyl or optionally substituted thiazolidinyl; or [symbol] is a single bond and Z is OH.

In some embodiments, the disease or injury is stroke (e.g., acute stroke or stroke in a recovery phase); congenital hypogonadotropic hypogonadism (e.g., Kallmann Syndrome); cerebral hemorrhage; traumatic brain injury (TBI); spinal cord injury (SCI); peripheral vascular disease (PVD); wounds; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection). In some embodiments, the disease or injury is congenital hypogonadotropic hypogonadism (e.g., Kallmann Syndrome); cerebral hemorrhage; traumatic brain injury (TBI); spinal cord injury (SCI); peripheral vascular disease (PVD); wounds; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection).

In some embodiments, the disease or injury is coronaviral infection.

In some embodiments, the disease or injury is stroke, provided that when Q is optionally substituted C₆-C₁₀ aryl, R₁ is H, Z is NR_{c}, and R_{c} is NR_{f}R_{g}, R_{f} and R_{g}, together with the nitrogen atom to which they are attached, do not form optionally substituted piperazinyl; when Z is NR_{c}, and R_{c} is NR_{f}R_{g}, one of R_{f} and R_{g} is H, and the other of R_{f} and R_{g} is C₁-C₆ alkyl substituted with one oxo, R_{g} is not further substituted with unsaturated heterocyclyl; piperazinyl; aryl; oxo; OR^{k}, wherein Rₖ is aryl or heterocyclyl; or NHRₗ, wherein Rₗ is aryl, cycloalkyl, or alkyl substituted with oxo; and when Q is optionally substituted C₆-C₁₀ aryl and Z is O, R₁ not C₁-C₆ alkyl substituted with NHRₘ, wherein Rₘ is aryl.

In a second aspect, the invention features a method of increasing spermatogenesis in a subject comprising administering to a subject a therapeutically effective amount of a compound, wherein the compound is a compound of formula (I):

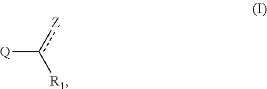

or a pharmaceutically acceptable salt or a tautomer thereof, in which Q is optionally substituted C₆-C₁₀ aryl or optionally substituted 6- to 10-membered heterocyclyl; R₁ is H, OH, optionally substituted C₁-C₆ alkyl, optionally substituted C₆-C₁₆ aryl, or optionally substituted 6- to 12-membered heteroaryl; and Z is O or NR_{c} and [symbol] is a double bond, wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein R_{d} and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, form an optionally substituted 6- to 10-membered heterocyclyl, or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol] is a single bond, and R₁ and Z, together with the carbon atom to which they are attached, form an optionally substituted oxazolidinyl or optionally substituted thiazolidinyl; or [symbol] is a single bond and Z is OH.

In some embodiments of the preceding aspects, the compound is a compound of formula (la):

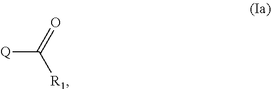

or a pharmaceutically acceptable salt thereof.

In some embodiments, R₁ is H, C₁-C₆ alkyl (e.g., methyl), or OH.

In some embodiments, R₁ is optionally substituted C₆-₁₆ aryl (e.g., phenyl). For example, R₁ is

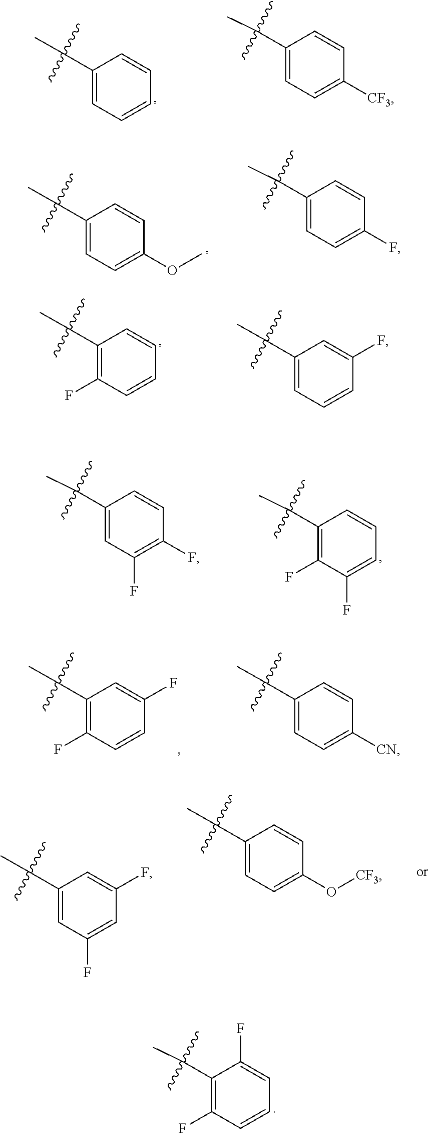

In some embodiments, R₁ is optionally substituted 6- to 12-membered heteroaryl. For example, R₁ is

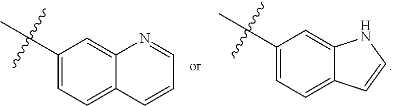 [SMILES: *c1ccc2cccnc2c1 ; *c1ccc2cc[nH]c2c1]

In some embodiments of the preceding aspects, the compound is a compound of formula (Ib):

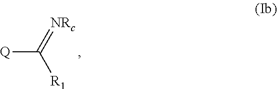

or a pharmaceutically acceptable salt or a tautomer thereof.

In some embodiments, R₁ is H.

In some embodiments, R_{c} is OR_{d}, e.g, OH.

In some embodiments, R_{c} is optionally substituted C₁-C₆ alkyl, e.g., methyl substituted with one or two optionally substituted C₆-C₁₆ aryl or C₁-C₁₅ heterocyclyl. For example, R_{c} is

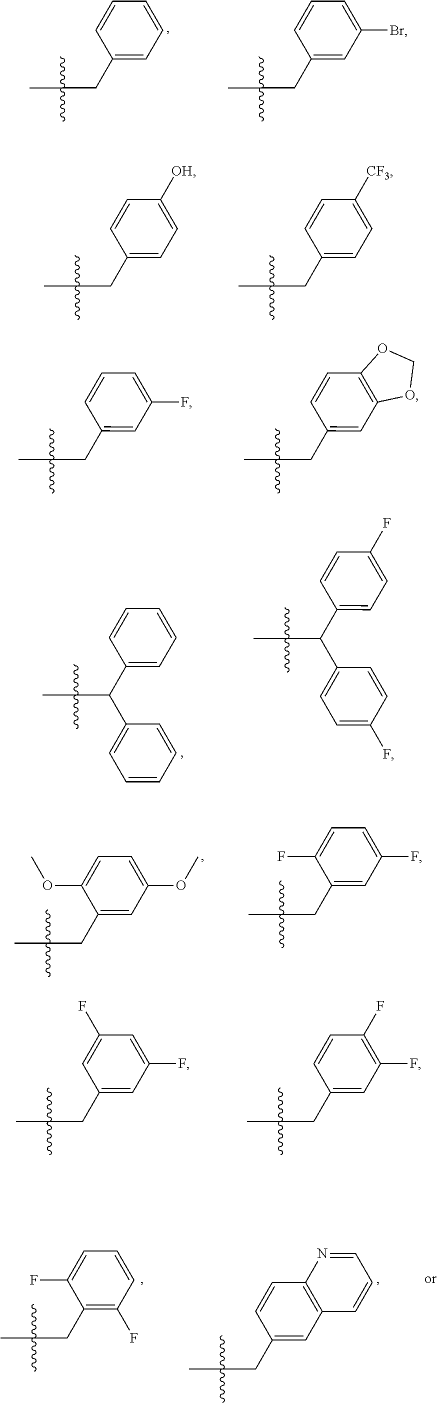

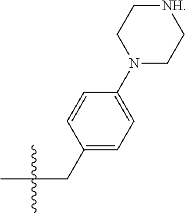 [SMILES: *Cc1ccc(N2CCNCC2)cc1]

In some embodiments, the compound is a compound of formula (Ib-1):

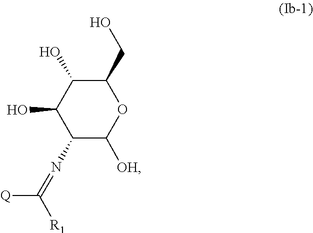

or a pharmaceutically acceptable salt or a tautomer thereof, wherein the tautomer of the compound of formula (Ib-1) is of formula:

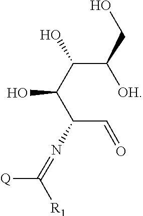

In some embodiments, R_{c} is optionally substituted C₆-C₁₆ aryl, e.g.,

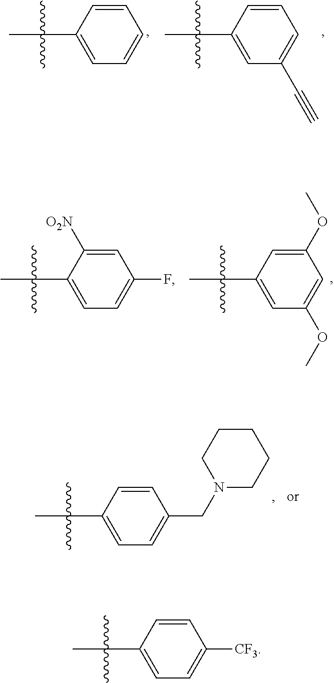

In some embodiments, R_{c} is optionally substituted C₁-C₁₅ heterocyclyl, e.g.,

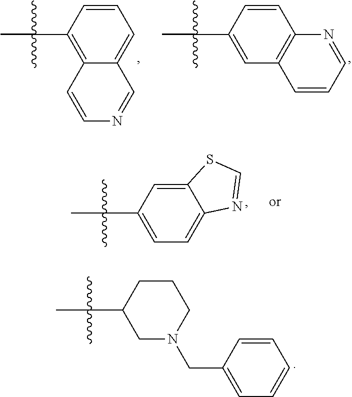

In some embodiments, R_{c} is optionally substituted C₄-C₁₃ cycloalkenyl, e.g.,

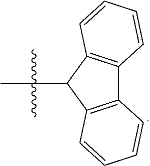 [SMILES: *C1c2ccccc2-c2ccccc21]

In some embodiments, R_{c} is NR_{f}R_{g}. In some embodiments, R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, In some embodiments, R_{c} is NH₂.

In some embodiments, R_{f} and R_{g} are independently H or optionally substituted C₆-C₁₆ aryl, wherein at least one of R_{f} and R_{g} is optionally substituted C₆-C₁₆ aryl. For example, R_{c} is

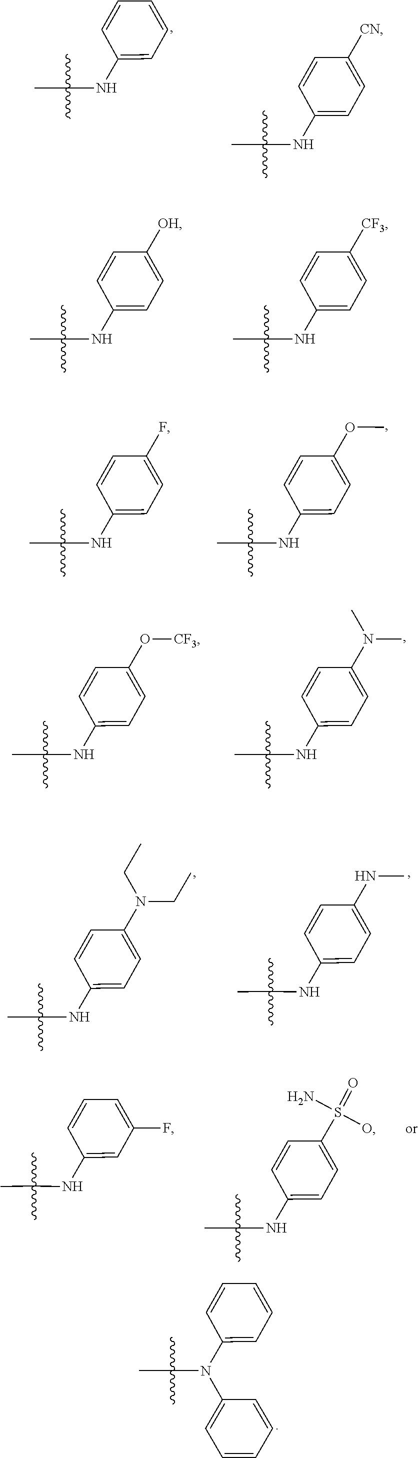

In some embodiments, R_{f} and R_{g} are independently H or optionally substituted C₁-C₆ alkyl, wherein at least one of R_{f} and R_{g} is optionally substituted C₁-C₆ alkyl. For example, at least one of R_{f} and R_{g} is C₁-C₆ alkyl substituted with oxo. In some embodiments, the compound is a compound of formula (Ib-2):

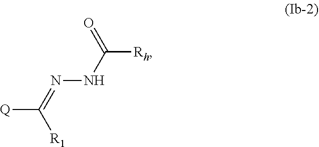

or a pharmaceutically acceptable salt thereof, wherein Rₕ is optionally substituted C₁-C₆ alkyl, optionally substituted C₃-C₈ cycloalkyl, optionally substituted C₆-C₁₆ aryl, or optionally substituted C₁-C₁₅ heterocyclyl.

In some embodiments, Rₕ is optionally substituted C₁-C₆ alkyl, e.g., CH₂N(CH₃)₂.

In some embodiments, Rₕ is optionally substituted C₃-C₈ cycloalkyl, e.g.,

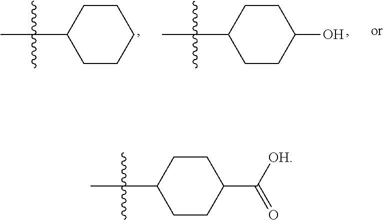 [SMILES: *C1CCCCC1 ; *C1CCC(O)CC1 ; *C1CCC(C(=O)O)CC1]

In some embodiments, Rₕ is optionally substituted C₆-C₁₄ aryl, e.g.,

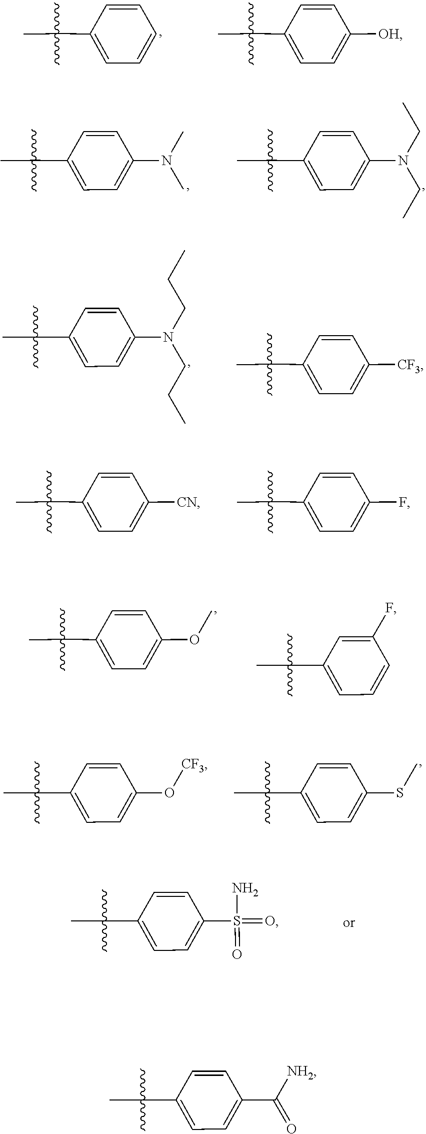

In some embodiments, Rₕ is optionally substituted C₁-C₁₅ heterocyclyl, e.g.,

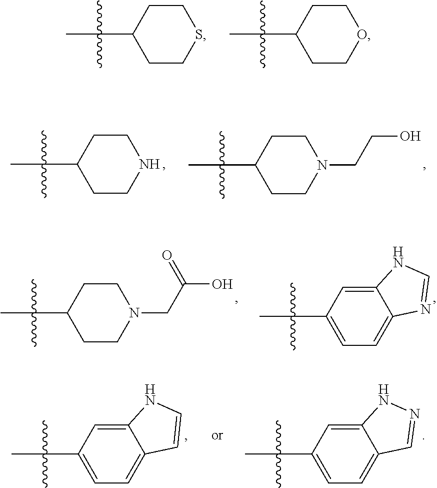

In some embodiments, R_{f} and R_{g} are independently H or optionally substituted C₃-C₈ cycloalkyl, wherein at least one of R_{f} and R_{g} is optionally substituted C₃-C₈ cycloalkyl. For example, R_{c} is

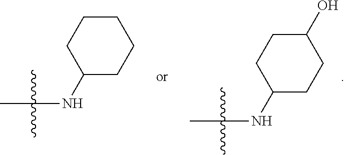 [SMILES: *NC1CCCCC1 ; *NC1CCC(O)CC1]

In some embodiments, R_{f} and R_{g} are independently H or optionally substituted C₁-C₁₅ heterocyclyl, wherein at least one of R_{f} and R_{g} is optionally substituted C₁-C₁₅ heterocyclyl. For example, R_{c} is

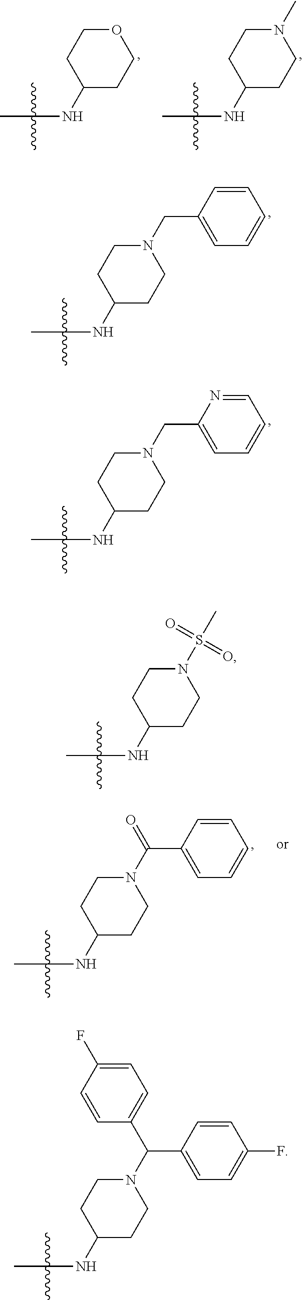

In some embodiments, R_{f} and R_{g}, together with the nitrogen atom to which they are attached, forms an optionally substituted 6- to 10-membered heterocyclyl. For example, R_{c} is

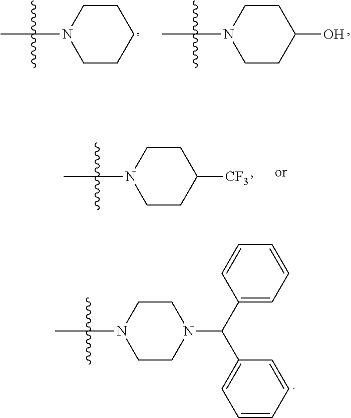 [SMILES: *N1CCCCC1 ; *N1CCC(O)CC1 ; *N1CCC(C(F)(F)F)CC1 ; *N1CCN(C(c2ccccc2)c2ccccc2)CC1]

In some embodiments, R_{c} is N═C(R₁′)Q′, e.g., wherein R₁′ is H and/or Q′ and Q are identical.

In some embodiments of the preceding aspects, [symbol] is a single bond, and R₁ and Z, together with the carbon atom to which they are attached, form an optionally substituted oxazolidinyl or optionally substituted thiazolidinyl. For example, R₁ and Z, together with the carbon atom to which they are attached, form

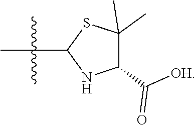 [SMILES: *C1N[C@@H](C(=O)O)C(C)(C)S1]

In some embodiments of the preceding aspects, [symbol] is a single bond and Z is OH.

In some embodiments of the preceding aspects, Q is

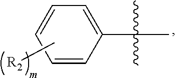

wherein each R₂ is independently halo or NRₐR_{b}, wherein Rₐ and R_{b} are independently H; optionally substituted C₁-C₆ alkyl; optionally substituted C₆-C₁₆ aryl; or SO₂Rᵢ, wherein Rᵢ is H or C₁-C₆ alkyl; or Rₐ and R_{b}, together with the nitrogen atom to which they are attached, forms an optionally substituted 5- to 10-membered heterocyclyl; and m is 0 to 5.

In some embodiments, m is 0.

In some embodiments, m is 1. For example, Q is

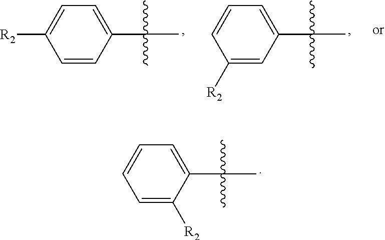

In some embodiments, R₂ is halo.

In some embodiments, R₂ is NRₐR_{b}.

In some embodiments, Rₐ and R_{b} are independently H or optionally substituted C₁-C₆ alkyl. For example, R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂. In some embodiments, R₂ is N(CH₂CH₃)₂.

In some embodiments, Rₐ and R_{b}, together with the nitrogen atom to which they are attached, forms an optionally substituted 5- to 10-membered heterocyclyl. For example, R₂ is

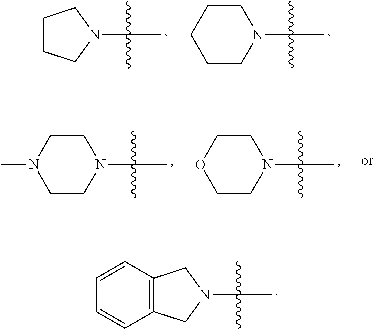

In some embodiments, Rₐ and R_{b} are independently H or optionally substituted C₆-C₁₆ aryl. For example, R₂ is

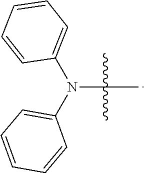 [SMILES: *N(c1ccccc1)c1ccccc1]

In some embodiments, m is 2. For example, Q is

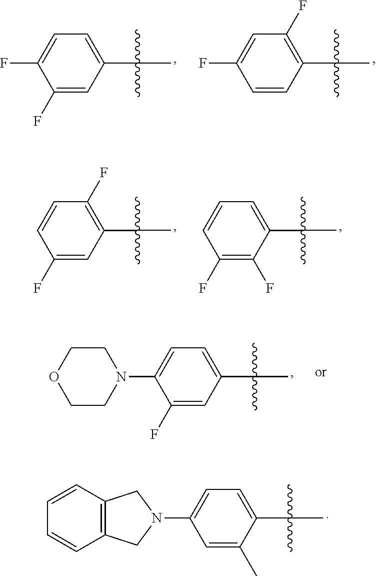 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1 ; *c1ccc(N2Cc3ccccc3C2)cc1C]

In some embodiments, Q is optionally substituted 6- to 10-membered heterocyclyl, e.g.,

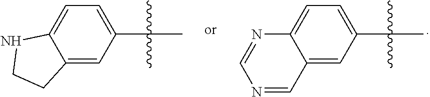 [SMILES: *c1ccc2c(c1)CCN2 ; *c1ccc2ncncc2c1]

In some embodiments of the preceding aspects, the compound is

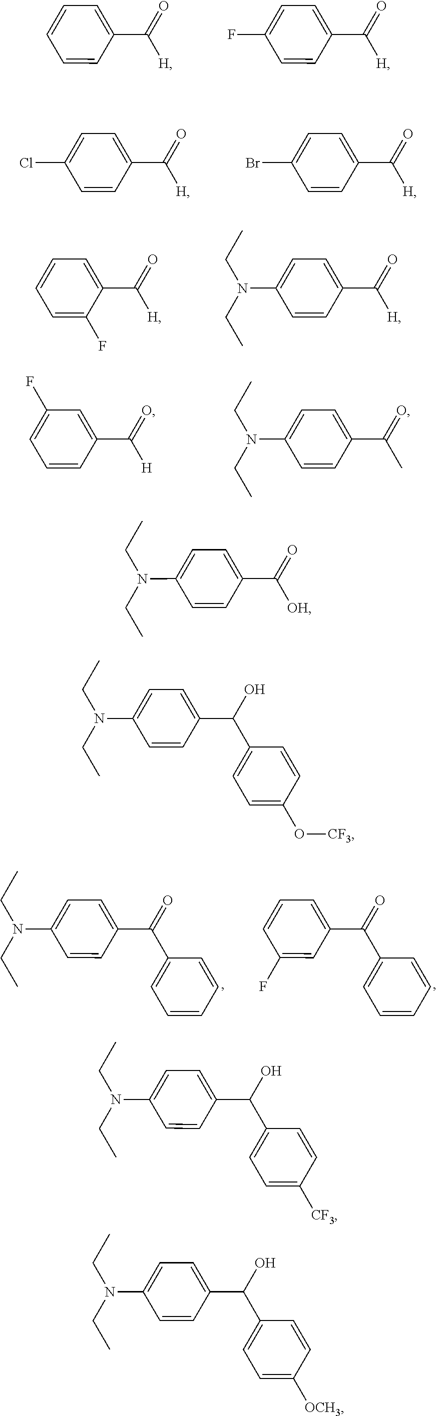

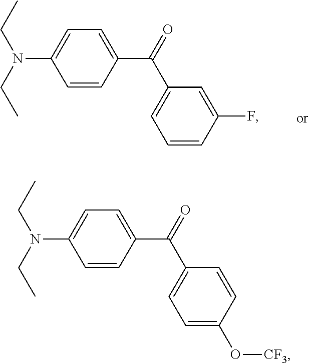 [SMILES: CCN(CC)c1ccc(C(=O)c2cccc(F)c2)cc1 ; CCN(CC)c1ccc(C(=O)c2ccc(OC(F)(F)F)cc2)cc1]

or a pharmaceutically acceptable salt thereof.

In some embodiments of the preceding aspects, the compound is:

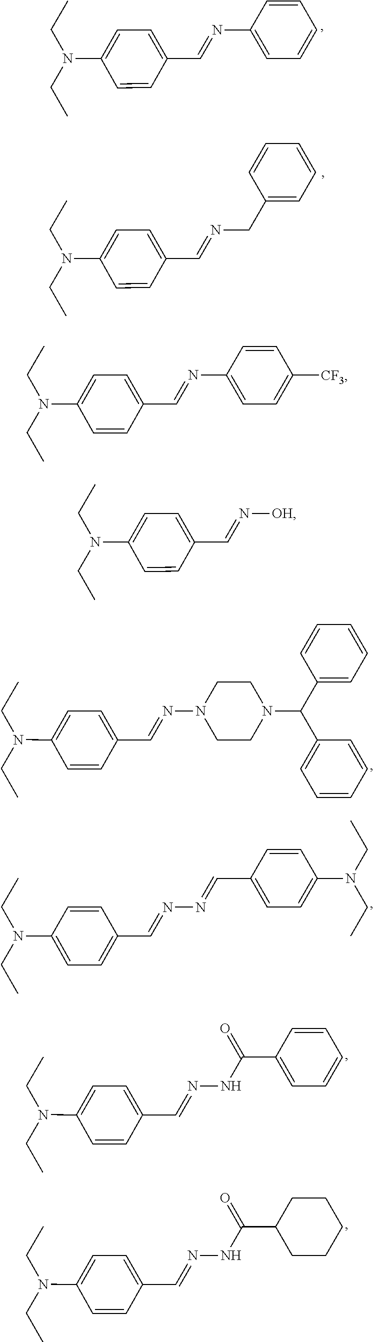

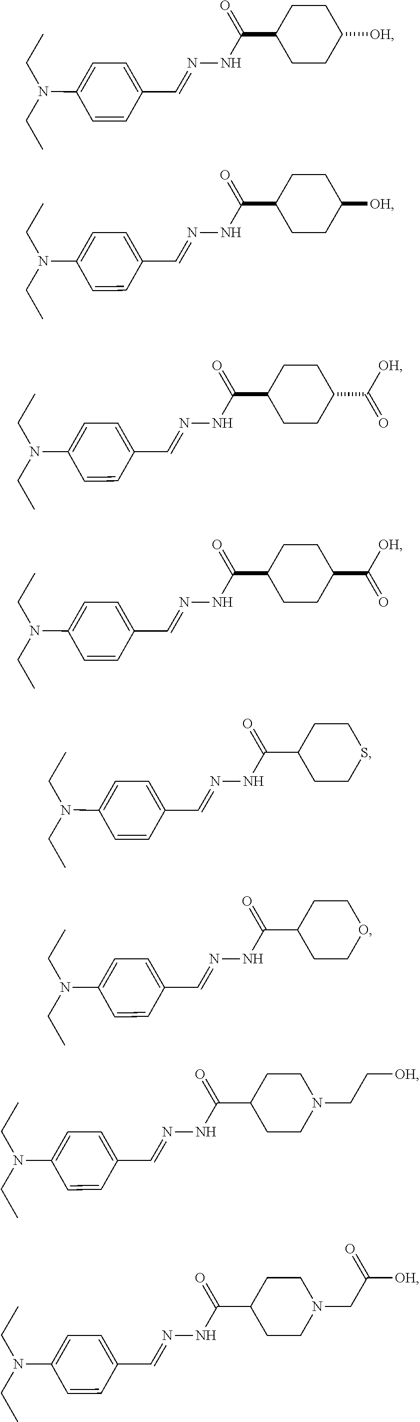

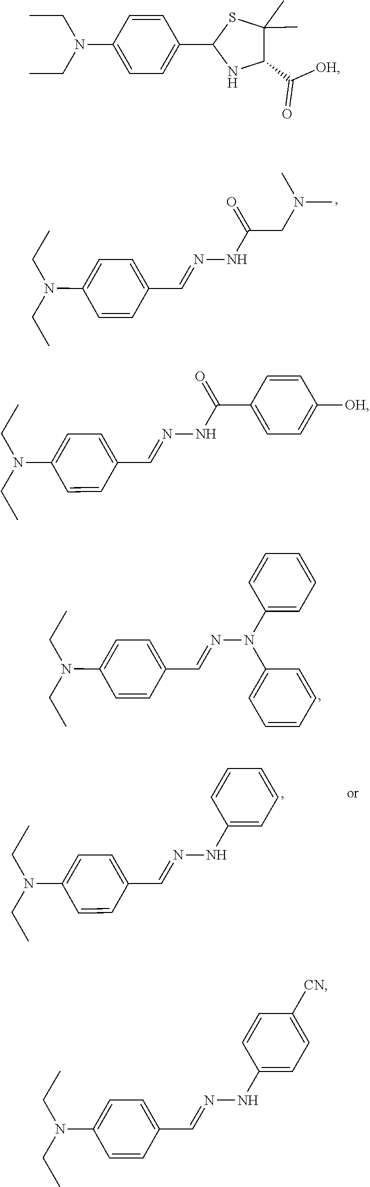

or a pharmaceutically acceptable salt thereof.

In a third aspect, the invention features a compound of formula (I′):

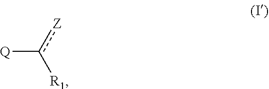

or a pharmaceutically acceptable salt or a tautomer thereof, in which Q is optionally substituted CB-C₁₀ aryl or optionally substituted 6- to 10-membered heterocyclyl; R₁ is H; and Z is NR_{c} and [symbol] is a double bond, wherein R_{c} is a group of formula:

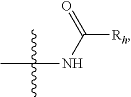

in which Rₕ is substituted C₃-C₈ cycloalkyl or optionally substituted C₁-C₁₅ heterocyclyl; or R_{c} is a group of formula N═C(R₁′)Q′, wherein R₁′ is H and Q′ is optionally substituted C₆-C₁₀ aryl or optionally substituted 6- to 10-membered heterocyclyl; or R_{c} is a group of formula:

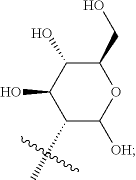

or [symbol] is a single bond, and R₁ and Z, together with the carbon atom to which they are attached, form an optionally substituted oxazolidinyl or optionally substituted thiazolidinyl; or [symbol] is a single bond and Z is OH.

In some embodiments, the compound is a compound of formula (Ib′):

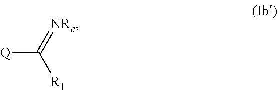

or a pharmaceutically acceptable salt or a tautomer thereof.

In some embodiments, the compound is a compound of formula (Ib′-1):

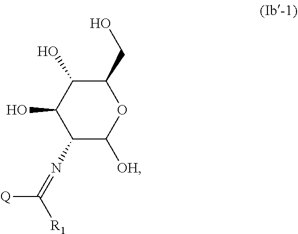

or a pharmaceutically acceptable salt or a tautomer thereof, wherein the tautomer of the compound of formula (Ib′-1) is of formula:

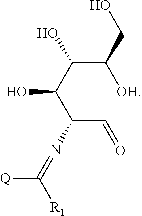

In some embodiments, the compound is a compound of formula (Ib′-2):

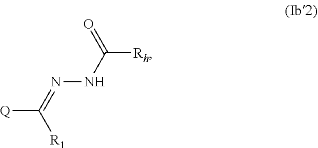

or a pharmaceutically acceptable salt thereof.

In some embodiments, Rₕ is C₃-C₈ cycloalkyl having at least one substituent, e.g.,

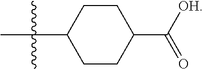 [SMILES: *C1CCC(C(=O)O)CC1]

In some embodiments, Rₕ is optionally substituted C₁-C₁₅ heterocyclyl, e.g.,

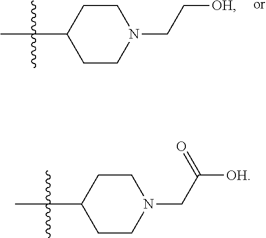 [SMILES: *C1CCN(CCO)CC1 ; "or" ; *C1CCN(CC(=O)O)CC1]

In some embodiments, R_{c} is N═C(R₁′)Q′. In some embodiments, R₁′ is H. In some embodiments, Q′ and Q are identical.

In some embodiments, R₁ and Z, together with the carbon atom to which they are attached, form an optionally substituted oxazolidinyl or optionally substituted thiazolidinyl. In some embodiments, R₁ and Z, together with the carbon atom to which they are attached, form

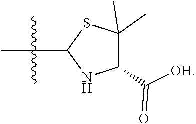 [SMILES: *C1N[C@@H](C(=O)O)C(C)(C)S1]

In some embodiments, [symbol] is a single bond and Z is OH.

In some embodiments, Q is

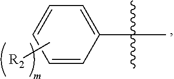

wherein each R₂ is independently halo or NRₐR_{b}, wherein Rₐ and R_{b} are independently H; optionally substituted C₁-C₆ alkyl; optionally substituted C8-C16 aryl; or SO₂Rᵢ, wherein Rᵢ is H or C₁-C₆ alkyl; or Rₐ and R_{b}, together with the nitrogen atom to which they are attached, forms an optionally substituted 5- to 10-membered heterocyclyl; and m is 0 to 5.

In some embodiments, m is 0.

In some embodiments, m is 1. For example, Q is

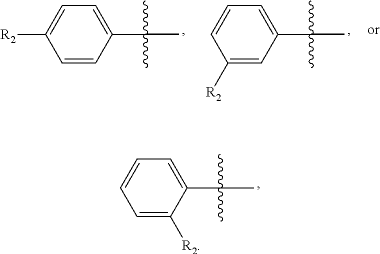

In some embodiments, R₂ is halo.

In some embodiments, R₂ is NRₐR_{b}.

In some embodiments, Rₐ and R_{b} are independently H or optionally substituted C₁-C₆ alkyl. For example, R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂. In some embodiments, R₂ is N(CH₂CH₃)₂.

In some embodiments, Rₐ and RD, together with the nitrogen atom to which they are attached, forms an optionally substituted 5- to 10-membered heterocyclyl. For example, R₂ is

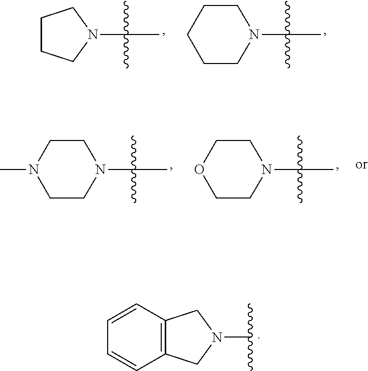

In some embodiments, Rₐ and R_{b} are independently H or optionally substituted C₆-C₁₆ aryl. For example, R₂ is

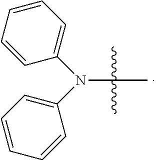 [SMILES: *N(c1ccccc1)c1ccccc1]

In some embodiments, m is 2. For example, Q is

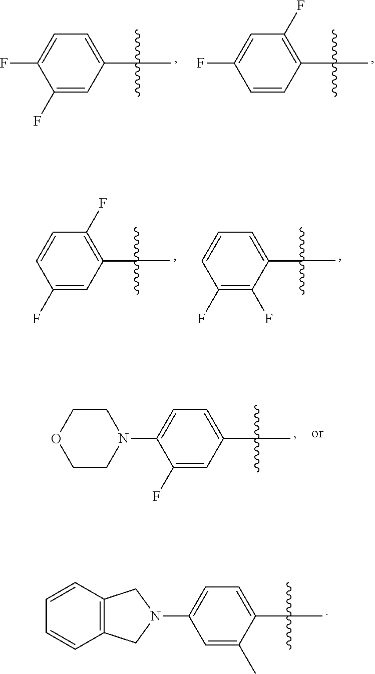 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1 ; *c1ccc(N2Cc3ccccc3C2)cc1C]

In some embodiments, Q is optionally substituted 6- to 10-membered heterocyclyl, e.g.,

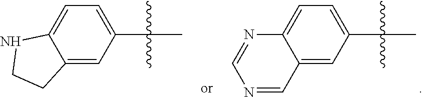 [SMILES: *c1ccc2c(c1)CCN2 ; *c1ccc2ncncc2c1]

In some embodiments, the compound is:

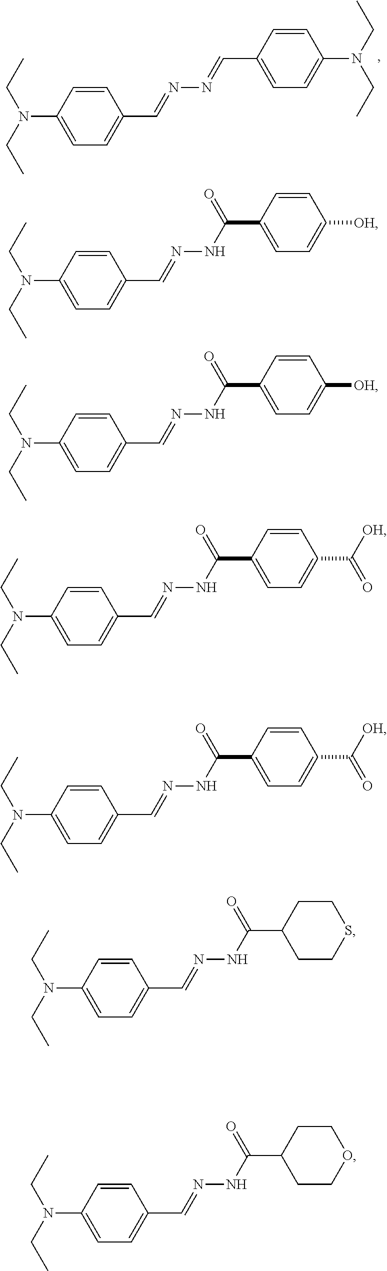

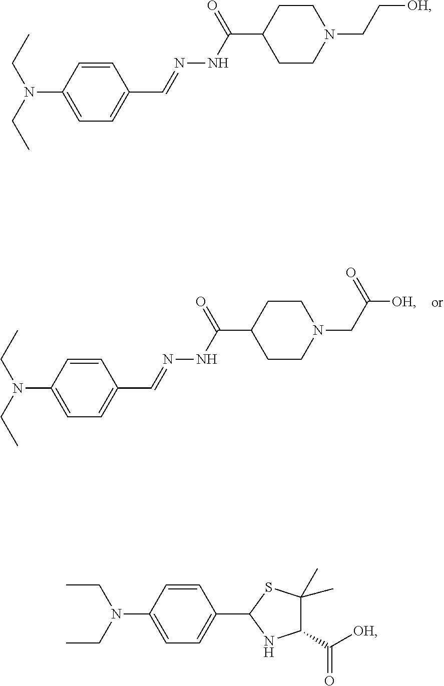 [SMILES: CCN(CC)c1ccc(/C=N/NC(=O)C2CCN(CCO)CC2)cc1 ; CCN(CC)c1ccc(/C=N/NC(=O)C2CCN(CC(=O)O)CC2)cc1 ; CCN(CC)c1ccc(C2N[C@@H](C(=O)O)C(C)(C)S2)cc1]

or a pharmaceutically acceptable salt thereof.

In a fourth aspect, the invention features pharmaceutical composition including a compound of formula (I′), (Ib′), (Ib′-1), or (Ib′-2), or a pharmaceutically acceptable salt thereof, and a pharmaceutically acceptable excipient.

In a fifth aspect, the invention features a pharmaceutical composition including a compound of formula (I):

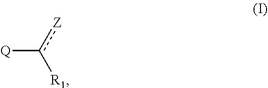

in which Q is optionally substituted C₆-C₁₀ aryl, or optionally substituted 6- to 10-membered heterocyclyl; R₁ is H, OH, optionally substituted C₁-C₆ alkyl, optionally substituted C₆-C₁₆ aryl or optionally substituted 6- to 12-membered heteroaryl; and Z is O or NR_{c}, and [symbol] is a double bond, wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein Rₐ and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, forms an optionally substituted 6- to 10-membered heterocyclyl, or or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol] is a single bond, and R₁ and Z, together with the carbon atom to which they are attached, form an optionally substituted oxazolidinyl or optionally substituted thiazolidinyl; or [symbol] is a single bond and Z is OH, or a pharmaceutically acceptable salt or a tautomer thereof, and a pharmaceutically acceptable excipient.

In some embodiments, the compound is a compound of formula (la): or a pharmaceutically acceptable salt thereof.

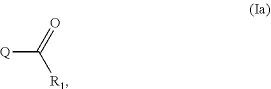

In some embodiments, R₁ is H or C₁-C₆ alkyl.

In some embodiments, R₁ is optionally substituted C₆-₁₆ aryl (e.g., phenyl). For example, R₁ is

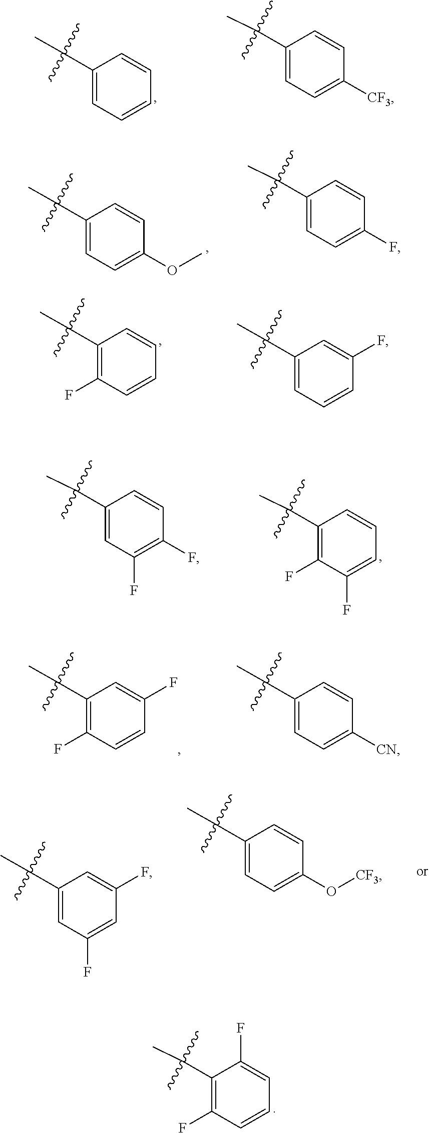

In some embodiments, R₁ is optionally substituted 6- to 12-membered heteroaryl. For example, R₁ is

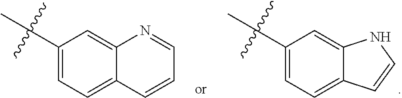 [SMILES: *c1ccc2cccnc2c1 ; *c1ccc2cc[nH]c2c1]

In some embodiments, the compound is a compound of formula (Ib):

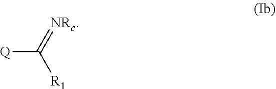

or a pharmaceutically acceptable salt or a tautomer thereof.

In some embodiments, R₁ is H.

In some embodiments, R_{c} is OR_{d}, e.g., OH.

In some embodiments, R_{c} is optionally substituted C₁-C₆ alkyl, e.g., methyl substituted with one or two optionally substituted C₆-C₁₆ aryl or C₁-C₁₅ heterocyclyl. For example, R_{c} is

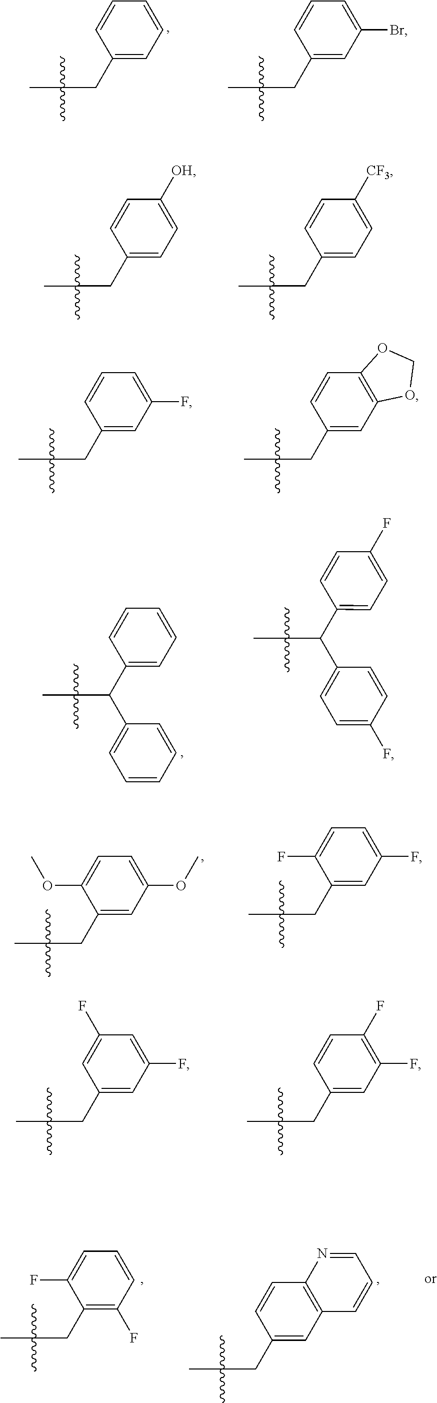

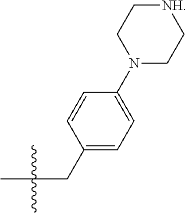 [SMILES: *Cc1ccc(N2CCNCC2)cc1]

In some embodiments, the compound is a compound of formula (Ib-1):

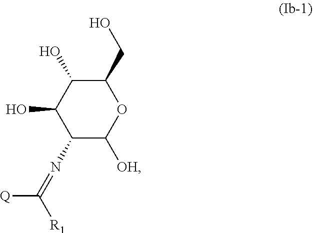

or a pharmaceutically acceptable salt or a tautomer thereof. The tautomer of the compound of formula (Ib-1) is of formula:

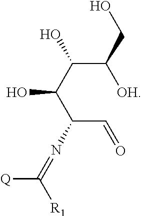

In some embodiments, R_{c} is optionally substituted C₆-C₁₆ aryl, e.g.,

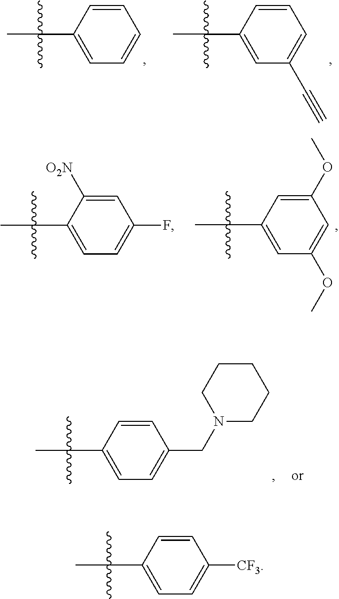

In some embodiments, R_{c} is optionally substituted C₁-C₁₅ heterocyclyl, e.g.,

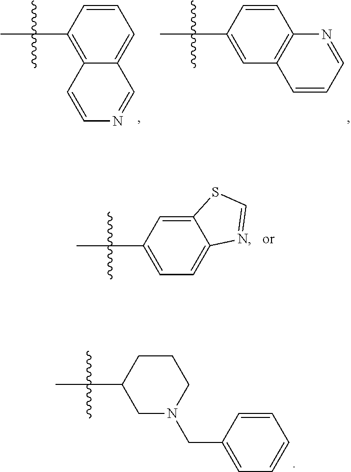 [SMILES: *c1cccc2cnccc12 ; *c1ccc2ncccc2c1 ; *c1ccc2ncsc2c1 ; *C1CCCN(Cc2ccccc2)C1]

In some embodiments, R_{c} is optionally substituted C₄-C₁₃ cycloalkenyl, e.g.,

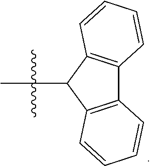 [SMILES: *C1c2ccccc2-c2ccccc21]

In some embodiments, R_{c} is NR_{f}R_{g}. In some embodiments, R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C18 aryl, In some embodiments, R_{c} is NH₂.

In some embodiments, R_{f} and R_{g} are independently H or optionally substituted C₆-C₁₆ aryl,

wherein at least one of R_{f} and R_{g} is optionally substituted C₆-C₁₆ aryl. For example, R_{c} is

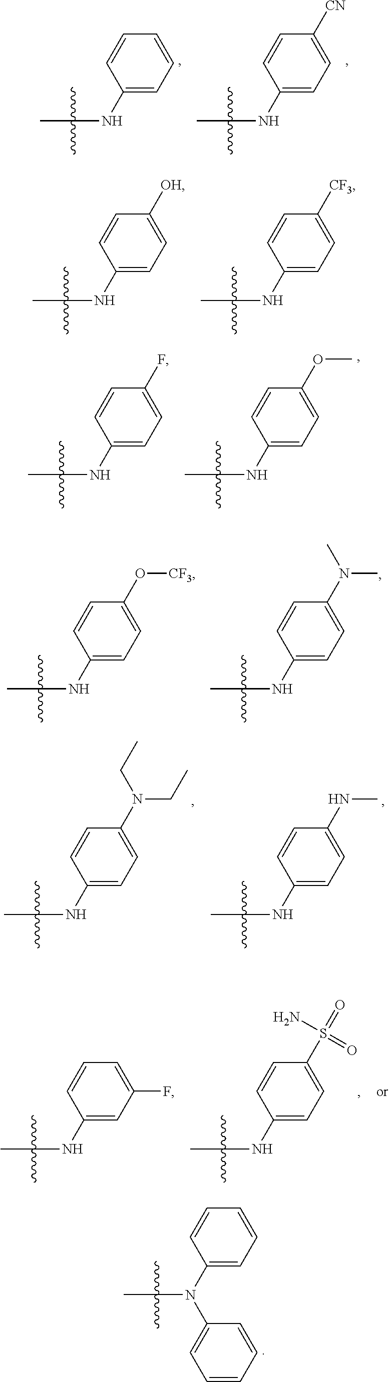

In some embodiments, R_{f} and R_{g} are independently H or optionally substituted C₁-C₆ alkyl, wherein at least one of R_{f} and R_{g} is optionally substituted C₁-C₆ alkyl. For example, at least one of R_{f} and R_{g} is C₁-C₆ alkyl substituted with oxo. In some embodiments, the compound is a compound of formula (Ib-2):

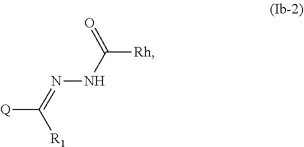

or a pharmaceutically acceptable salt thereof, wherein Rₕ is optionally substituted C₁-C₆ alkyl, optionally substituted C₃-C₈ cycloalkyl, optionally substituted C₆-C₁₆ aryl, or optionally substituted C₁-C₁₅ heterocyclyl.

In some embodiments, Rₕ is optionally substituted C₁-C₆ alkyl, e.g., CH₂N(CH₃)₂.

In some embodiments, Rₕ is optionally substituted C₃-C₈ cycloalkyl, e.g.,

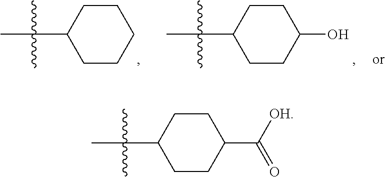 [SMILES: *C1CCCCC1 ; *C1CCC(O)CC1 ; *C1CCC(C(=O)O)CC1]

In some embodiments, Rₕ is optionally substituted C₆-C₁₄ aryl, e.g.,

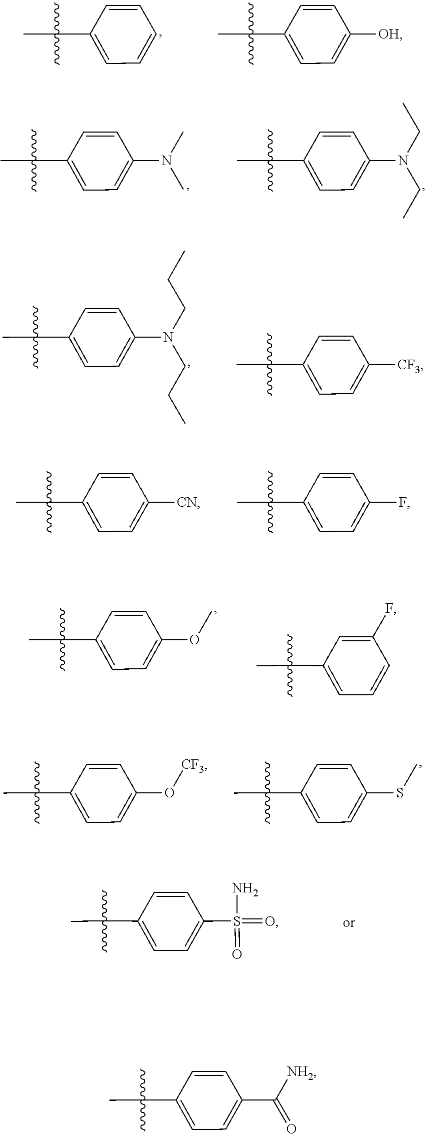

In some embodiments, Rₕ is optionally substituted C₁-C₁₅ heterocyclyl, e.g.,

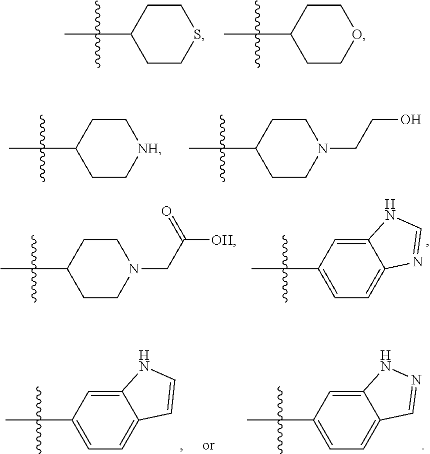

In some embodiments, R_{f} and R_{g} are independently H or optionally substituted C₃-C₈ cycloalkyl, wherein at least one of R_{f} and R_{g} is optionally substituted C₃-C₈ cycloalkyl. For example, R_{c} is

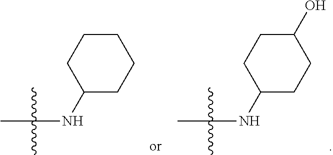 [SMILES: *NC1CCCCC1 ; *NC1CCC(O)CC1]

In some embodiments, R_{f} and R_{g} are independently H or optionally substituted C₁-C₁₅ heterocyclyl, wherein at least one of R_{f} and R_{g} is optionally substituted C₁-C₁₅ heterocyclyl. For example, R_{c} is

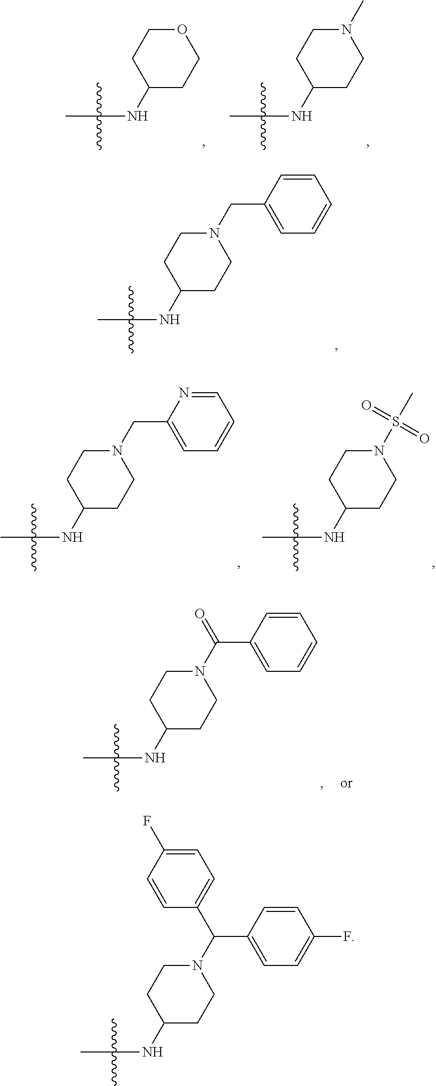

In some embodiments, R_{f} and R_{g}, together with the nitrogen atom to which they are attached, forms an optionally substituted 6- to 10-membered heterocyclyl. For example, R_{c} is

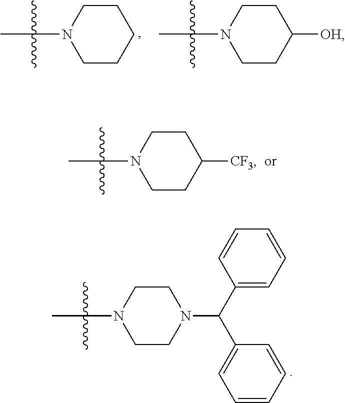

In some embodiments, R_{c} is N═C(R₁′)Q′, e.g., wherein R₁′ is H and/or Q′ and Q are identical.

In some embodiments of the preceding aspects, [symbol] is a single bond, and R₁ and Z, together with the carbon atom to which they are attached, form an optionally substituted oxazolidinyl or optionally substituted thiazolidinyl. For example, R₁ and Z, together with the carbon atom to which they are attached, form

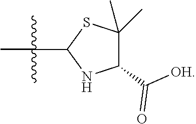 [SMILES: *C1N[C@@H](C(=O)O)C(C)(C)S1]

In some embodiments of the preceding aspects, [symbol] is a single bond and Z is OH.

In some embodiments, Q is

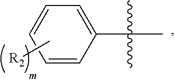

wherein each R₂ is independently halo or NRₐR_{b}, wherein Rₐ and R_{b} are independently H; optionally substituted C₁-C₆ alkyl; optionally substituted C₆-C₁₆ aryl; or SO₂Rᵢ, wherein Rᵢ is H or C₁-C₆ alkyl; or Rₐ and R_{b}, together with the nitrogen atom to which they are attached, forms an optionally substituted 5- to 10-membered heterocyclyl; and m is 0 to 5.

In some embodiments, m is 0.

In some embodiments, m is 1. For example, Q is

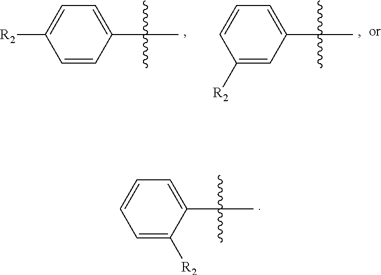

In some embodiments, R₂ is halo.

In some embodiments, R₂ is NRₐR_{b}.

In some embodiments, Rₐ and R_{b} are independently H or optionally substituted C₁-C₆ alkyl. For example, R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂. In some embodiments, R₂ is N(CH₂CH₃)₂.

In some embodiments, Rₐ and R_{b}, together with the nitrogen atom to which they are attached, forms an optionally substituted 5- to 10-membered heterocyclyl. For example, R₂ is

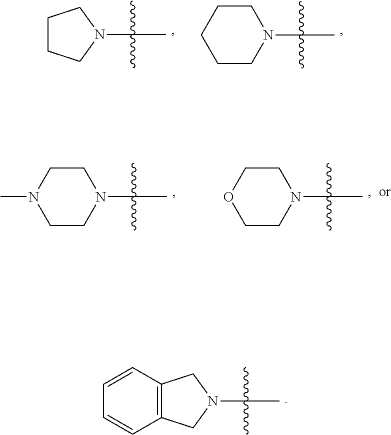

In some embodiments, Rₐ and R_{b} are independently H or optionally substituted C₆-C₁₆ aryl. For

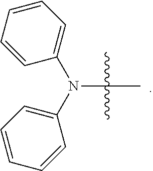 [SMILES: *N(c1ccccc1)c1ccccc1]

example, R₂ is

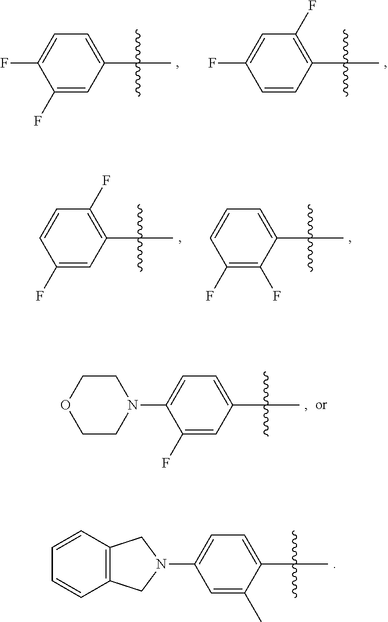 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1 ; *c1ccc(N2Cc3ccccc3C2)cc1C]

In some embodiments, m is 2. For example, Q is

In some embodiments, Q is optionally substituted 6- to 10-membered heterocyclyl, e.g.,

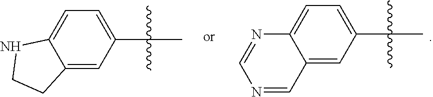 [SMILES: *c1ccc2c(c1)CCN2 ; *c1ccc2ncncc2c1]

In some embodiments of the preceding aspects, the compound is

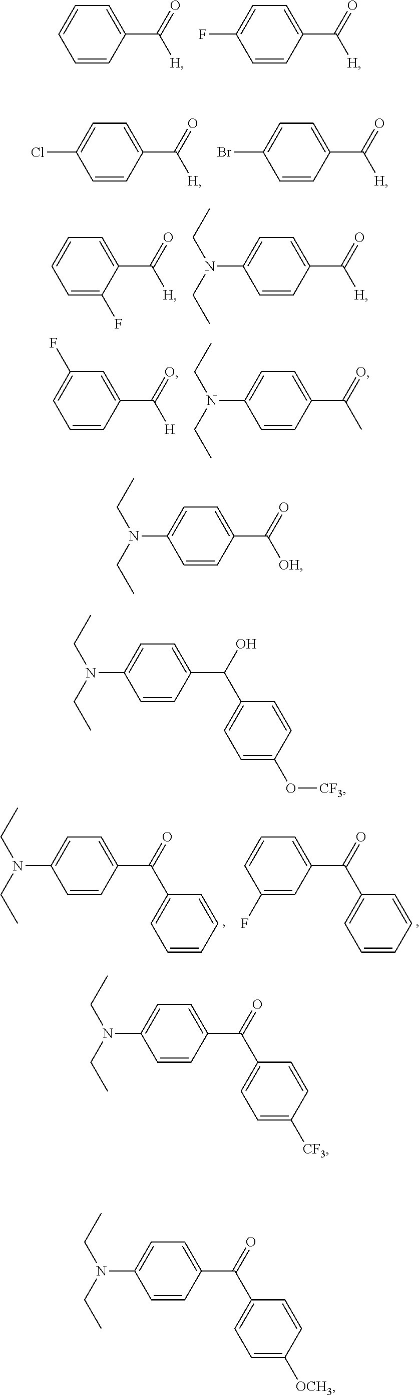

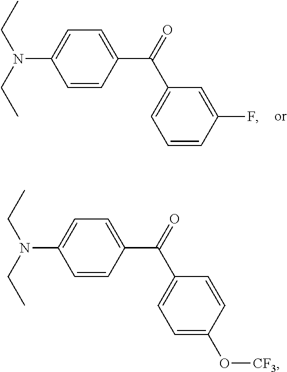 [SMILES: CCN(CC)c1ccc(C(=O)c2cccc(F)c2)cc1 ; CCN(CC)c1ccc(C(=O)c2ccc(OC(F)(F)F)cc2)cc1]

or a pharmaceutically acceptable salt thereof.

In some embodiments of the preceding aspects, the compound is:

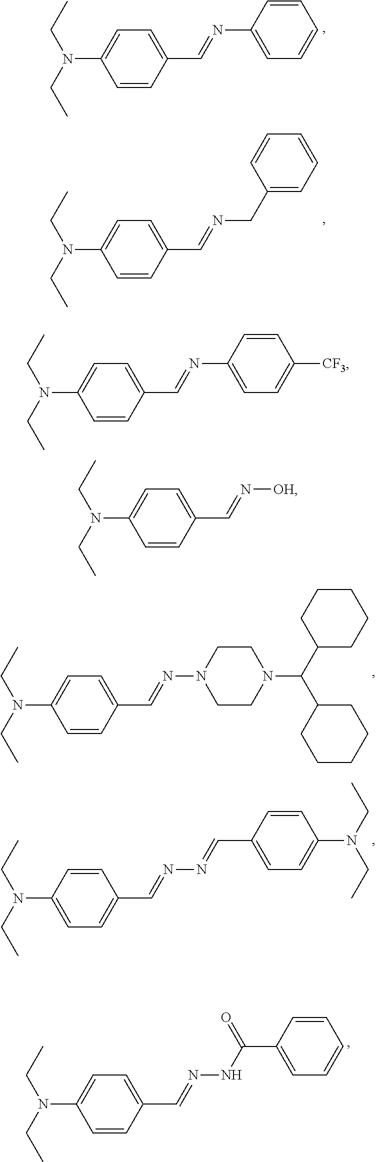

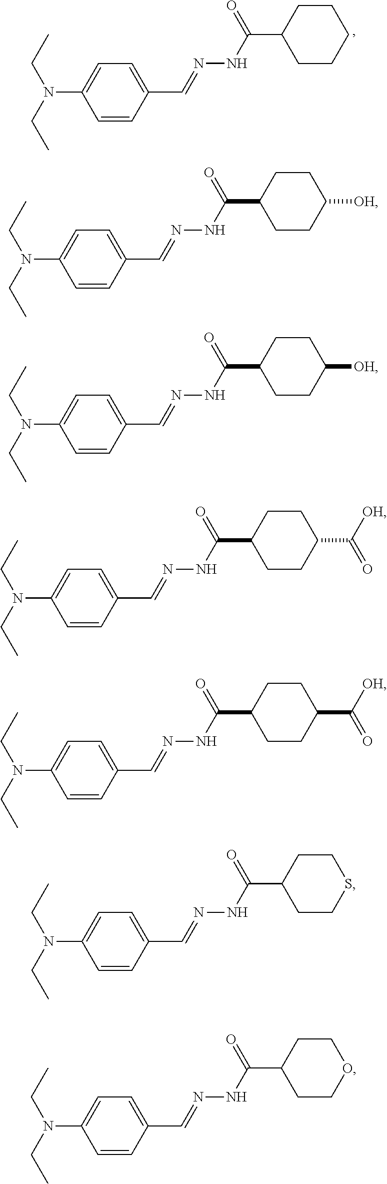

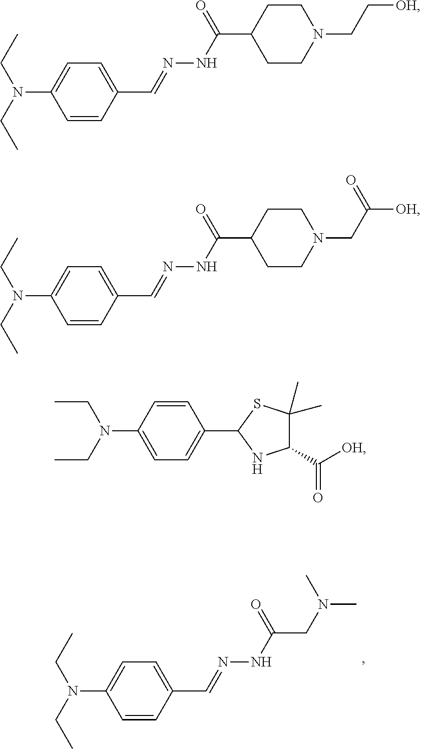 [SMILES: CCN(CC)c1ccc(/C=N/NC(=O)C2CCN(CCO)CC2)cc1 ; CCN(CC)c1ccc(/C=N/NC(=O)C2CCN(CC(=O)O)CC2)cc1 ; CCN(CC)c1ccc(C2N[C@@H](C(=O)O)C(C)(C)S2)cc1 ; CCN(CC)c1ccc(/C=N/NC(=O)CN(C)C)cc1]

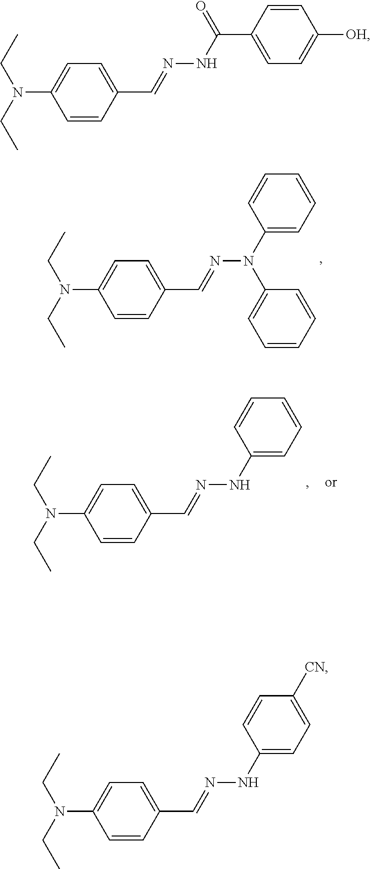 [SMILES: CCN(CC)c1ccc(/C=N/NC(=O)c2ccc(O)cc2)cc1 ; CCN(CC)c1ccc(/C=N/N(c2ccccc2)c2ccccc2)cc1 ; CCN(CC)c1ccc(/C=N/Nc2ccccc2)cc1 ; CCN(CC)c1ccc(/C=N/Nc2ccc(C#N)cc2)cc1]

or a pharmaceutically acceptable salt thereof.

In some embodiments, the pharmaceutical compositions is for use in the treatment of a disease or an injury in a subject. In some embodiments, the disease or injury is stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection). In certain embodiments, the disease or injury is stroke, e.g., acute stroke and/or stroke in a recovery phase. In other embodiments, the disease or injury is congenital hypogonadotropic hypogonadism, e.g., Kallmann Syndrome. In other embodiments, the disease or injury is viral infection (e.g., coronaviral infection).

In some embodiments, the disease or injury is stroke, provided that when Q is optionally substituted C₆-C₁₀ aryl, Rᵢ is H, Z is NR_{c}, and R_{c} is NR_{f}R_{g}, R_{f} and R_{g}, together with the nitrogen atom to which they are attached, do not form optionally substituted piperazinyl; when Z is NR_{c}, and R_{c} is NR_{f}R_{g}, one of R_{f} and R_{g} is H, and the other of R_{f} and R_{g} is C₁-C₆ alkyl substituted with one oxo, R_{g} is not further substituted with unsaturated heterocyclyl; piperazinyl; aryl; oxo; OR^{k}, wherein Rₖ is aryl or heterocyclyl; or NHR_{I}, wherein Rᵢ is aryl, cycloalkyl, or alkyl substituted with oxo; and when Q is optionally substituted C₆-C₁₀ aryl and Z is O, R₁ not C₁-C₆ alkyl substituted with NHRₘ, wherein Rₘ is aryl.

In some embodiments, the disease or injury is for use in increasing spermatogenesis in a subject.

### DEFINITIONS

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

As used herein, the term “therapeutically effective amount” refers to an amount sufficient to effect beneficial or desired results, such as clinical results, and, as such, a “therapeutically effective amount” depends upon the context in which it is being applied. For example, in the context of administering a compound disclosed herein (e.g., a compounds of any one of formulas (I), (Ib), (Ib-1), (Ib-2), (I′), (Ib′), and (Ib′-2) and Table 9) to treat or enhance a subject's recovery from a stroke or TBI, a therapeutically effective amount of a compound is, for example, an amount sufficient to alleviate or reverse the effect of the stroke or TBI. For example, the subject may regain lost motor functions due to the stroke or TBI.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms or conditions; diminishment of extent of disease, disorder, or condition; stabilizing (i.e., not worsening) state of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “subject,” as used herein, can be a human, non-human primate, or other mammal, such as but not limited to dog, cat, horse, cow, pig, goat, monkey, rat, mouse, and sheep.

As used herein, the term “pharmaceutical composition” refers to an active compound, formulated together with one or more pharmaceutically acceptable excipients. In some embodiments, a compound of the invention is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-20, 1-18, 1-16, 1-14, 1-12, 1-10, 1-8, 1-6, 1-4, or 1-2 carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl.

The term “alkylene,” as used herein, refers to a divalent radical obtained by removing a hydrogen atom from a carbon atom of an alkyl group. The divalency of an alkylene group does not include the optional substituents on the alkylene group. Examples of alkylene groups include, but are not limited to, methylene, ethylene, and n-propylene.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkynyl,” as used herein, refers to a branched or straight-chain monovalent unsaturated aliphatic radical containing at least one carbon-carbon triple bond and only C and H when unsubstituted. Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-20, 2-18, 2-16, 2-14, 2-12, 2-10, 2-8, 2-6, or 2-4 carbon atoms (e.g., C₂-C₂₀, C₂-C₁₈, C₂-C₁₆, C₂-C₁₄, C₂-C₁₂, C₂-C₁₀, C₂-C₈, C₂-C₆, or C₂-C₄). Examples include, but are not limited to, ethynyl, 1-propynyl, and 3-butynyl.

The term “aryl,” as used herein, refers to any monocyclic or fused ring bicyclic or multicyclic system containing only carbon atoms in the ring(s), which has the characteristics of aromaticity in terms of electron distribution throughout the ring system, e.g., phenyl, naphthyl, or phenanthryl. An aryl group may have, e.g., six to sixteen carbons (e.g., six carbons, ten carbons, thirteen carbons, fourteen carbons, or sixteen carbons).

The term “cycloalkyl,” as used herein, represents a monovalent, saturated cyclic group containing only C and H when unsubstituted. A cycloalkyl may have, e.g., three to twenty carbons (e.g., a C₃-C₇, C₃-C₈, C₃-C₉, C₃-C₁₀, C₃-C₁₁, C₃-C₁₂, C₃-C₁₄, C₃-C₁₆, C₃-C₁₈, or C₃-C₂₀ cycloalkyl). Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spiro-cyclic compounds.

The term “cycloalkenyl,” as used herein, represents a monovalent, unsaturated carbocyclic ring system that includes at least one carbon-carbon double bond, only C and H when unsubstituted, and is not fully aromatic. A cycloalkenyl may have, e.g., four to twenty carbons (e.g., a C₄-C₇, C₄-C₈, C₄-C₉, C₄-C₁₀, C₄-C₁₁, C₄-C₁₂, C₄-C₁₃, C₄-C₁₄, C₄-C₁₆, C₄-C₁₈, or C₄-C₂₀ cycloalkenyl). Exemplary cycloalkenyl groups include, but are not limited to, cyclopentenyl, cyclohexenyl, and cycloheptenyl. The term “cycloalkenyl” also includes cyclic groups having a bridged multicyclic structure in which one or more carbons bridges two non-adjacent members of a monocyclic ring, e.g., bicyclo[2.2.2]oct-2-ene. The term “cycloalkenyl” also includes fused bicyclic and multicyclic nonaromatic, carbocyclic ring systems containing one or more double bonds, e.g., fluorene.

The term “halo,” as used herein, refers to a fluorine (fluoro), chlorine (chloro), bromine (bromo), or iodine (iodo) radical.

The term “heterocyclyl,” as used herein, represents a monocyclic or fused ring bicyclic or multicyclic system having at least one heteroatom as a ring atom. For example, a heterocyclyl ring may have, e.g., one to fifteen carbons ring atoms (e.g., a C₁-C₂, C₁-C₃, C₁-C₄, C₁-C₅, C₁-C₆, C₁-C₇, C₁-C₈, C₁-C₉, C₁-C₁₀, C₁-C₁₁, C₁-C₁₂, C₁-C₁₃, C₁-C₁₄, or C₁-C₁₅ heterocyclyl) and one or more (e.g., one, two, three, four, or five) ring heteroatoms independently selected from the group consisting of nitrogen, oxygen, and sulfur. Heterocyclyl groups may or may not include a ring that is aromatic. An aromatic heterocyclyl group is referred to as a “heteroaryl” group. In preferred embodiments of the invention, a heterocyclyl group is a 3- to 8-membered ring, a 3- to 6-membered ring, a 4- to 6-membered ring, a 6- to 10-membered ring, a 6- to 12-membered ring, a 5-membered ring, or a 6-membered ring. Exemplary 5-membered heterocyclyl groups may have zero to two double bonds, and exemplary 6-membered heterocyclyl groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocyclyl groups include, for example, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocyclyl groups include optionally substituted azepine, optionally substituted 1,4-diazepine, optionally substituted thiepine, and optionally substituted 1,4-thiazepine.

The term “heterocyclylene,” as used herein, refers to a divalent radical obtained by removing a hydrogen from a ring atom from a heterocyclyl group. The divalency of a heterocyclylene group does not include the optional substituents on the heterocyclylene group.

The term “oxo,” as used herein, refers to a divalent oxygen atom represented by the structure ═O.

The phrase “optionally substituted X,” as used herein, is intended to be equivalent to “X, wherein X is optionally substituted” (e.g., “alkyl, wherein said alkyl is optionally substituted”). It is not intended to mean that the feature “X” (e.g. alkyl) per se is optional. The term “optionally substituted,” as used herein, refers to having 0, 1, or more substituents (e.g., 0-25, 0-20, 0-10, or 0-5 substituents).

Alkyl, alkylene, alkenyl, alkynyl, cycloalkyl, cycloalkenyl, aryl, heterocyclyl, and heterocyclylene groups may be substituted with cycloalkyl; cycloalkenyl; aryl; heterocyclyl; halo; OR^{a}, wherein R^{a} is H, alkyl, alkenyl, alkynyl, cycloalkyl, cycloalkenyl, aryl, or heterocyclyl; SR^{a}, wherein R^{a} is as defined herein; CN; NO₂; N₃; NR^{b}R^{c}; wherein each of R^{b} and R^{c} is, independently, H, alkyl, alkenyl, alkynyl, cycloalkyl, cycloalkenyl, aryl, or heterocyclyl; SO₂R^{d}, wherein Rd is H, alkyl or aryl; SO₂NR^{e}R_{f}, wherein each of R^{e} and R^{f} is, independently, H, alkyl, or aryl; SOR^{g}, wherein R^{g} is H, alkyl, or aryl; or SiR^{h}Rⁱ, wherein R^{h} and Rⁱ is, independently, H or alkyl. Aryl, cycloalkyl, cycloalkenyl, heteroaryl, and heterocyclyl groups may also be substituted with alkyl, alkenyl, or alkynyl. Alkyl, alkylene, alkenyl, alkynyl, cycloalkyl, cycloalkenyl, heterocyclyl, and heterocyclylene groups may also be substituted with oxo or =NR^{j}, wherein Rᵢ is H or alkyl. In some embodiments, a substituent is further substituted as described herein. For example, a C₁ alkyl group, i.e., methyl, may be substituted with oxo to form a formyl group and further substituted with —OH or —NH₂ to form a carboxyl group or an amido group.

### DESCRIPTION OF THE DRAWINGS

FIG. 1 is a graph showing the thermal stability assay (TSA) data of purified FGF-2.FGFR₁ complex with and without the addition of Compound 1o (dotted line: without Compound 1o; solid line: 25 μM Compound 1o).

FIG. 2 is a graph showing the phosphorylation of FGFR₁ in the presence of increasing concentrations of Compound 1o in a cell-based system.

FIG. 3 is a graph showing the behavioral score of rats in a forelimb placing test pre-middle cerebral artery occlusion (MCAO) and post-MCAO (treated with Compound 1o or vehicle).

FIG. 4 is a graph showing the behavioral score of rats in a hindlimb placing test pre-MCAO and post-MCAO (treated with Compound 1o or vehicle).

FIG. 5 is a graph showing the right swing % of rats in a body swing test pre-MCAO and post-MCAO (treated with Compound 1o or vehicle).

FIG. 6 is a graph showing the body weight of rats pre-MCAO and post-MCAO (treated with Compound 1o or vehicle).

FIG. 7 is a graph showing the cell survival of HAP1 cells infected with human coronavirus 229E following a 4-day incubation period in the presence of Compound 1o. Compound 1o (0.002 μM, 0.008 μM, 0.04 μM, 0.2 μM, or 1 uM) and FGF-2 (1 ng/mL) were added on Day -1, Day 0, Day 1, and Day 2 of infection by human coronavirus 229E.

### DETAILED DESCRIPTION OF THE INVENTION

The invention features compounds, compositions, and methods for treating various diseases, disorders, and other medical conditions, for example, stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection), by administering a compound disclosed herein (e.g., a compound of any one of formulas (I), (Ib), (Ib-1), (Ib-2), (I′), (Ib′), (Ib′-1), and (Ib′-2) or a compound of Table 9) to the subject. Without wishing to be bound by theory, the compounds are believed to modulate FGF activity, e.g., by enhancing the binding between FGF-2 and its receptors, e.g., FGF-R1. Preferably, methods of the invention are directed to enhancing a subject's recovery from brain injuries and diseases, such as cerebrovascular diseases, e.g., stroke (such as stroke recovery) and TBI.

### Compounds

The compounds for treating FGF-modulated diseases or injuries disclosed herein include compounds of formula (I):

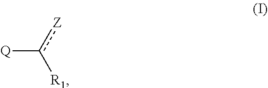

or a pharmaceutically acceptable salt or a tautomer thereof, wherein

Q is optionally substituted C₆-C₁₀ aryl or optionally substituted 6- to 10-membered heterocyclyl;

R₁ is H, OH, optionally substituted C₁-C₆ alkyl, optionally substituted C₆-C₁₆ aryl, or optionally substituted 6- to 12-membered heteroaryl; and

Z is O or NR_{c} and [symbol] is a double bond,

wherein R_{c} is H; optionally substituted C₁-C₆ alkyl; optionally substituted C₁-C₆ alkenyl; optionally substituted C₁-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein Rₐ and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, form an optionally substituted 6- to 10-membered heterocyclyl, or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or

[symbol] is a single bond, and R₁ and Z, together with the carbon atom to which they are attached, form an optionally substituted oxazolidinyl or optionally substituted thiazolidinyl; or

[symbol] is a single bond, and Z is OH.

Exemplary compounds for the treatment of FGF-modulated diseases or injuries are shown in the Example 1 and Tables 1-3 and 5-9,

### Pharmaceutical Compositions

A pharmaceutical composition of the invention contains one or more of the compounds disclosed herein (e.g., one or more of the compounds of any one of formulas (I), (Ib), (Ib-1), (Ib-2), (I′), (Ib′), (Ib′-1), and (Ib′-2) or Table 9) as the therapeutic compound. In addition to a therapeutically effective amount of the compound, the pharmaceutical compositions also contain a pharmaceutically acceptable excipient, which can be formulated by methods known to those skilled in the art. In some embodiments, pharmaceutical compositions for treating FGF-modulated diseases contain one or more of the compounds disclosed herein (e.g., one or more of the compounds of any one of formulas (I), (Ib), (Ib-1), (Ib-2), (I′), (Ib′), (Ib′-1), and (Ib′-2) or Table 9) and one or more exogenous ligands, e.g., exogenous FGF-2. The compounds disclosed herein (e.g., the compounds of formulas (I), (Ib), (Ib-1), (Ib-2), (I′), (Ib′), (Ib′-1), and (Ib′-2) and Table 9) may also be administered with or without other therapeutics for a particular condition.

The compounds disclosed herein (e.g., the compounds of formulas (I), (Ib), (Ib-1), (Ib-2), (I′), (Ib′), (Ib′-1), and (Ib′-2) and Table 9) may be used in the form of free base, or in the form of salts, solvates, and as prodrugs. All forms are within the scope of the invention.

Exemplary routes of administration of the pharmaceutical compositions (or the compounds of the composition) include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

### Formulations for Oral Administration

The pharmaceutical compositions of the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

### Formulations for Parenteral Administration

The pharmaceutical compositions of the invention can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference in its entirety.

The parenteral formulation can be any of the five general types of preparations identified by the USP-NF as suitable for parenteral administration:
(1) “Drug Injection:” a liquid preparation that is a drug substance (e.g., a compound of the invention), or a solution thereof;
(2) “Drug for Injection:” the drug substance (e.g., a compound of the invention) as a dry solid that will be combined with the appropriate sterile vehicle for parenteral administration as a drug injection;
(3) “Drug Injectable Emulsion:” a liquid preparation of the drug substance (e.g., a compound of the invention) that is dissolved or dispersed in a suitable emulsion medium;
(4) “Drug Injectable Suspension:” a liquid preparation of the drug substance (e.g., a compound of the invention) suspended in a suitable liquid medium; and
(5) “Drug for Injectable Suspension:” the drug substance (e.g., a compound of the invention) as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropyl cellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP 43 NF38), published in 2019.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Methods of Treatment

The compounds disclosed herein (e.g., the compounds of formulas (I), (Ib), (Ib-1), (Ib-2), (I′), (Ib′), (Ib′-1), and (Ib′-2) and Table 9) are, in general, suitable for any therapeutic use, e.g., where modulation of FGF activity is desired. In some embodiments, compounds disclosed herein (e.g., the compounds of formulas (I), (Ib), (Ib-1), (Ib-2), (I′), (Ib′), (Ib′-1), and (Ib′-2) and Table 9) may be used to treat any disease or disorder that may benefit from increased activity of FGF, for example, stroke, e.g., acute stroke and/or stroke in a recovery phase; congenital hypogonadotropic hypogonadism (e.g., Kallmann Syndrome); cerebral hemorrhage; traumatic brain injury (TBI); spinal cord injury (SCI); peripheral vascular disease (PVD); wounds, i.e., for wound healing; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection (e.g., coronaviral infection).

Increased activity of FGF, e.g., FGF-2, has beneficial effects in cardiovascular, cerebrovascular, and peripheral vascular disease, including enhancement of functional recovery after stroke (Wada et al. Stroke 2003; 34:2724; Kawamata et al. Proc. Natl. Acad. Sci. USA 1997; 94:8179;) and TBI (Dietrich et al. Journal of Neurotrauma 1996; 13:309; McDermott et al. Journal of Neurotrauma 1997; 14:191). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance a subject's recovery from brain injuries and diseases, preferably cerebrovascular diseases, e.g., stroke and TBI, and conditions associated therewith (e.g., anosmia associated with TBI).

In particular, the compounds, pharmaceutical compositions, and methods of the invention may be used to enhance the recovery of subjects who had suffered a brain injury or disease, e.g., stroke or TBI. In some embodiments, the stroke may be an acute stroke. In some embodiments, the stroke may be an acute ischemic stroke. In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat acute stroke by administering the compounds disclosed herein (e.g., the compounds of formulas (I), (Ib), (Ib-1), (Ib-2), (I′), (Ib′), (Ib′-1), and (Ib′-2) and Table 9) to a stroke subject within the first day after the stroke. In other embodiments, the compounds disclosed herein (e.g., the compounds of formulas (I), (Ib), (Ib-1), (Ib-2) , (I′), (Ib′), (Ib′-1), and (Ib′-2) and Table 9) may be used to treat and/or enhance functional recovery after stroke, i.e., stroke in a recovery phase, by administering the compounds disclosed herein (e.g., the compounds of formulas (I), (Ib), (Ib-1), (Ib-2), (I′), (Ib′), (Ib′-1), and (Ib′-2) and Table 9) to a stroke subject more than one day (e.g., days to years) after the stroke.

FGF may be used in the treatment of neurological diseases because of its neuroprotective properties and effects on neuronal proliferation (see, e.g., Katsouri et al. Neurobiol. Aging. 2015; 36(2): 821-31; Kiyota et al. Proc. Natl. Acad. Sci. 2011; 108(49): E1339-48; Ma et al. Curr. Pharm. Des. 2007; 13(15): 1607-16; and Woodbury et al. J. Neuroimmune Pharmacol. 2014; 9(2): 92-101). In some embodiments, the compounds of disclosed herein (e.g., the compounds of formulas (I), (Ib), (Ib-1), (Ib-2) , (I′), (Ib′), (Ib′-1), and (Ib′-2) and Table 9) may be used to treat or enhance recovery from neurological diseases, e.g., Alzheimer's disease, Parkinson's disease, and ALS . In yet other embodiments, the compounds of disclosed herein (e.g., the compounds of formulas (I), (Ib), (Ib-1), (Ib-2), (I′), (Ib′), (Ib′-1), and (Ib′-2) and Table 9) may be used to treat or enhance recovery from diseases, disorders, or medical symptoms related to memory disturbance.

FGF has been shown to be neuroprotective and therapeutic for hearing loss (see, e.g., D'Sa et al. Eur J Neurosci. 2007; 26:666-80; Zhang et al. Lin Chuang Er Bi Yan Hou Ke Za Zhi. 2002; 16:603-4; Zhai et al. Acta Otolaryngol. 2004; 124:124-9; Wimmer et al. Otol Neurotol. 2004; 25:33-40; Sekiya et al. Neurosurgery. 2003; 52:900-7; Smith et al. Hear Res. 2002; 169:1-12; Zhai et al. Zhonghua Er Bi Yan Hou Ke Za Zhi. 199; 32:354-6). Accordingly, the compounds of disclosed herein (e.g., the compounds of formulas (I), (Ib), (Ib-1), (Ib-2), (I′), (Ib′), (Ib′-1), and (Ib′-2) and Table 9) may be used to treat or prevent hearing loss.

FGF has been shown to modulate affective and addictive disorders (Turner et al. Neuron 2012; 76:160; Turner et al. Brain Res. 2008; 1224:63-68). In some preferred embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from diseases, disorders, or medical symptoms related to PTSD, anxiety, or depression. In other preferred embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from diseases, disorders, or medical symptoms related to substance abuse.

FGF has been shown to induce proliferation of progenitor and stem cells (Wada et al. Stroke 2003; 34:2724) and enhance axon regeneration (Haenzi et al. Neural Plasticity. 2017: 2740768). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to induce stem cell proliferation and differentiation, e.g., in the brain. The compounds disclosed herein (e.g., the compounds of formulas (I), (Ib), (Ib-1), (Ib-2), (I′), (Ib′), (Ib′-1), and (Ib′-2) and Table 9) may also be used to induce stem cell proliferation and differentiation, preferably stem cell proliferation and differentiation in the brain. Similarly, in some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from peripheral nerve injury or lesion and heart disease. In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from cerebral hemorrhage and spinal cord injury.

FGF has been shown to induce bone and cartilage formation and repair (Aspenberg et al. Acta Orthop Scand. 1989; 60:473-6; Chuma et al. Osteoarthritis Cartilage. 2004; 12:834-42). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from diseases and disorders related to bone and cartilage formation or to aid bone and cartilage formation. In some embodiments, the compounds disclosed herein (e.g., the compounds of formulas (I), (Ib), (Ib-1), (Ib-2), (I′), (Ib′), (Ib′-1), and (Ib′-2) and Table 9) may be used to induce wound healing.

FGF-2 has been shown to promote in vivo muscle regeneration in murine muscular dystrophy (Lefaucheur et al. Neuroscience Letters. 1995; 202: 121-124). In some embodiments, the compounds disclosed herein (e.g., the compounds of formulas (I), (Ib), (Ib-1), (Ib-2) (I′), (Ib′), (Ib′-1), and (Ib′-2) and Table 9) may be used to treat muscular dystrophy in a subject.

FGF has also been shown to promote hematopoiesis (Zhao et al. Blood. 2012; 120:1831). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to induce hematopoiesis. Hematopoiesis includes, but is not limited to, hematopoiesis in the brain and the bone marrow. The compounds disclosed herein (e.g., the compounds of formulas (I), (Ib), (Ib-1), (Ib-2), (I′), (Ib′), (Ib′-1), and (Ib′-2) and Table 9) may also be used to induce hematopoiesis, e.g., hematopoiesis in the brain and the bone marrow.

Mutations in FGFR₁ that cause loss or reduction of function have been implicated in several conditions including hypogonadotropic hypogonadism or conditions (e.g., Kallmann syndrome, anosmia, and normosmic idiopathic hypogonadotropic hypogonadism; see, e.g., Valdes-Socin et al. Front. Endocrinol. 2014; 5: 109 and Miraoui et al., Mol. Cell. Endocrinol. 2011; 346(1-2): 37-43). Such mutations result in reduced tyrosine kinase activity, cell surface expression, and/or reduced affinity for FGF (Pitteloud et al. Proc. Natl. Acad. Sci. USA 2006; 103:6281-67286; Raivio et al. J Clin. Endocrinol. Metab. 2009, 94:4380-4390). Increasing signaling via FGFR₁ may therefore treat hypogonadotropic hypogonadism (e.g., Kallmann syndrome, and normosmic idiopathic hypogonadotropic hypogonadism) and conditions associated therewith (e.g., anosmia). The compounds disclosed herein (e.g., the compounds of formulas (I), (Ib), (Ib-1), (Ib-2), (I′), (Ib′), (Ib′-1), and (Ib′-2) and Table 9) may also be used to increase signaling activity of FGFR₁ and enhance the binding between FGFR₁ and its ligands, thereby treating hypogonadotropic hypogonadism (e.g., Kallmann syndrome, and normosmic idiopathic hypogonadotropic hypogonadism) and conditions associated therewith (e.g., anosmia).

FGF affords protective effects on ischemia induced retinal injury (Unoki et al. Invest Ophthalomol. Vis. Sci. 1994; 35:907-915). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat or enhance recovery from an ocular arterial occlusive disorder, e.g., non-arteritic anterior ischemic optic neuropathy (NAION) or retinal artery occlusion.

The impairment of alveolar formation is the prominent feature of bronchopulmonary dysplasia, and FGF signaling is critical for alveologenesis (Bourbon et al., Pediatr. Res. 2005; 57: 38-46). In some embodiments, the compounds disclosed herein (e.g., the compounds of formulas (I), (Ib), (Ib-1), (Ib-2), (I′), (Ib′), (Ib′-1), and (Ib′-2) and Table 9) may also be used to enhance FGF signaling, thereby treating bronchopulmonary dysplasia.

The aging process has been associated with cellular senescence and a decline in somatic stem cell numbers and self-renewal within multiple tissues (Coutu et al. Aging. 2011; 3:920-933). FGFs and FGFRs are key regulators of both senescence and self-renewal in a variety of stem cell types. In some embodiments, the compounds disclosed herein (e.g., the compounds of formulas (I), (Ib), (Ib-1), (Ib-2), (I′), (Ib′), (Ib′-1), and (Ib′-2) and Table 9) may be used to modulate FGF signaling, thereby counteracting the effects of aging.

FGF has been shown to be crucial for the development of the vertebrate olfactory epithelium (OE) and the maintenance of OE neurogenesis during prenatal development (Kawauchi et al. Development. 2006; 132(23): 5211-23) and has also been shown to effect recovery of neural anosmia in mice by facilitating olfactory neuron regeneration (Nota et al. JAMA Otolaryngol. Head Neck Surg. 2013;

139: 398). In some embodiments, the compounds disclosed herein (e.g., the compounds of formulas (I), (Ib), (Ib-1), (Ib-2), (I′), (Ib′), (Ib′-1), and (Ib′-2) and Table 9) may be used for treating anosmia (e.g., anosmia associated with impaired olfactory neuron development or regeneration, olfactory neuron degeneration, or death of olfactory neurons).

FGF has been shown to inhibit viral replication (van Asten et al. J. Virol. 2018; 92:e00260-18). In some embodiments, the compounds disclosed herein (e.g., the compounds of formulas (I), (Ib), (Ib-1), (Ib-2), (I′), (Ib′), (Ib′-1), and (Ib′-2) and Table 9) may be used to treat a viral infection (e.g., coronaviral infection).

FGF signaling has been shown to increase spermatogenesis (Cotton et al. J. Cell. Sci. 20016; 119: 75-84; Saucedo et al. J Cell Physiol. 2018; 233(12): 9640-9651. In some embodiments, the compounds disclosed herein (e.g., the compounds of formulas (I), (Ib), (Ib-1), (Ib-2), (I′), (Ib′), (Ib′-1), and (Ib′-2) and Table 9) may be used to increase spermatogenesis in a subject.

The dosage of the pharmaceutical compositions of the invention depends on factors including the route of administration, the disease to be treated, and physical characteristics, e.g., age, weight, and general health, of the subject. Typically, the amount of a compound disclosed herein (e.g., a compound of any one of formulas (I), (Ib), (Ib-1), (Ib-2), (I′), (Ib′), (Ib′-1), and (Ib′-2) and Table 9) contained within a single dose may be an amount that effectively treats the disease without inducing significant toxicity. A pharmaceutical composition of the invention may include a dosage of a compound disclosed herein (e.g., a compound of any one of formulas (I), (Ib), (Ib-1), (Ib-2), (I′), (Ib′), (Ib′-1), and (Ib′-2) and Table 9) ranging from 0.001 to 500 mg/kg/day and, in a more specific embodiment, about 0.1 to about 100 mg/kg/day and, in a more specific embodiment, about 0.3 to about 30 mg/kg/day. The dosage may be adapted by the clinician in accordance with conventional factors such as the extent of the disease and different parameters of the subject. Typically, a pharmaceutical composition of the invention can be administered in an amount from about 0.001 mg up to about 500 mg/kg/day (e.g., 0.05, 0.01, 0.1, 0.2, 0.3, 0.5, 0.7, 0.8, 1 mg, 2 mg, 3 mg, 4 mg, 5 mg, 10 mg, 15 mg, 20 mg, 30 mg, 50 mg, 100 mg, 250 mg, or 500 mg) of a compound disclosed herein (e.g., a compound of any one of formulas (I), (Ib), (Ib-1), (Ib-2), (I′), (Ib′), (Ib′-1), and (Ib′-2) and Table 9).

Pharmaceutical compositions of the invention that contain a compound disclosed herein (e.g., a compound of any one of formulas (I), (Ib), (Ib-1), (Ib-2), (I′), (Ib′), (Ib′-1), and (Ib′-2) and Table 9) may be administered to a subject in need thereof, e.g., subjects who had suffered a brain injury or disease, e.g., a stroke or TBI, one or more times (e.g., 1-10 times or more) daily, weekly, monthly, biannually, annually, or as medically necessary. Preferably, the compounds disclosed herein (e.g., the compounds of formulas (I), (Ib), (Ib-1), (Ib-2), (I′), (Ib′), (Ib′-1), and (Ib′-2) and Table 9) may be administered on at least two consecutive days, e.g., on at least 3 consecutive days. Dosing on multiple days may be particularly beneficial in stroke recovery. Preferably, a subject may be administered a therapeutically effective amount of a compound disclosed herein (e.g., a compound of any one of formulas (I), (Ib), (Ib-1), (Ib-2), (I′), (Ib′), (Ib′-1), and (Ib′-2) and Table 9) or a pharmaceutical composition of the invention within the first month (e.g., within 30, 25, 20, 15, 10, 5, or 1 day) after onset of disease or injury, e.g., stroke or TBI. Preferably, a subject may be administered a therapeutically effective amount of a compound disclosed herein (e.g., a compound of any one of formulas (I), (Ib), (Ib-1), (Ib-2), (I′), (Ib′), (Ib′-1), and (Ib′-2) and Table 9) or a pharmaceutical composition of the invention immediately (e.g., within hours) after disease or injury, e.g., stroke or TBI. The timing between administrations may decrease as the medical condition improves or increase as the health of the subject declines.

### EXAMPLES

### Example 1. Compound Preparation

The general procedures used to synthesize the compounds are described in reaction Schemes 1-4 and are illustrated in the examples below. The following examples are put forth so as to provide those of ordinary skill in the art with a complete disclosure and description of how to make and use the present invention and are not intended to limit the scope of the invention, nor are they intended to represent that the experiments below were performed or that they are all of the experiments that may be performed. It is to be understood that exemplary description written in the present tense were not necessarily performed, but rather that the descriptions can be performed to generate data and the like of a nature described therein. Synthesized compounds were analyzed and characterized by use of the following equipment: Liquid chromatography-mass spectra (LC/MS) were obtained using an Agilent LC/MSD G1946D or an Agilent 1100 Series LC/MSD Trap G1311A or G2435A. Quantifications were obtained on a Cary 50 Bio UV-visible spectrophotometer. ¹H, ¹³C, and ¹⁹F nuclear magnetic resonance (NMR) spectra were obtained using a Varian INOVA NMR spectrometer at 400, 100, and 376 MHz, respectively. High-performance liquid chromatography (HPLC) analytical separations were performed on an Agilent 1100 or Agilent 1200 HPLC analytical system and followed by an Agilent Technologies G1315B Diode Array Detector set at or near the UVmax 169 210 nm. HPLC preparatory separations were performed on a Gilson preparative HPLC system or an Agilent 1100 preparative HPLC system and followed by an Agilent Technologies G1315B Diode Array Detector set at or near the UVmax@ 210 nm. Analytical chiral HPLC separations were performed on an Agilent 1100 analytical system and followed by an Agilent Technologies G1315B Diode Array Detector set at or near the UVmax@ 210 nm. The separations were accomplished with a Gemini 3 μm or 5 μm C_{18 50×2.5} mm or 250×4.6 mm solid-phase column eluting with acetic acid-methanol-water gradient or ammonium acetate-acetonitrile-water gradient. Flash chromatography was performed using CombiFlash NextGen 300+using RediSep Silica columns. All final compounds gave satisfactory purity (≥95%) by HPLC and by ¹H NMR spectroscopy. Thin-layer chromatography (TLC) analyses are performed on Uniplate 250 um silica gel plates (Analtech, Inc. Catalog no. 02521) and were typically developed for visualization by UV/Vis, using 50 vol % concentrated sulfuric acid in water spray, iodine stain, or Hanessian's stain.

### ABBREVIATIONS

In describing the invention, chemical elements are identified in accordance with the Periodic Table of Elements. Abbreviations and symbols utilized herein are in accordance with the common usage of such abbreviations and symbols by those skilled in the chemical arts. The following abbreviations are used herein:
ACNacetonitrile
AcOEt ethyl acetate
AcOH acetic acid
APCI atmospheric pressure chemical ionization
Boc tert-butoxycarbonyl
DCM dichloromethane
DIPEA diisopropylamine
DMAP 4-dimethylamino pyridine
DMSO-d6 deuterated dimethylsulfoxide
DMSO dimethylsulfoxide
EtOH ethanol
Et₂NH diethylamine
g gram(s)
Hep heptane
Hex hexane
h hours
H₂O water
HPLC high pressure liquid chromatography
I₂ lodine
i-PrOH isopropanol
MeOH methanol
MgSO₄ magnesium sulfate
min minutes
mg milligram(s)
mmol millimolar
mol mole
MTBE methyl tert-butyl ether
MW microwave
N₂ nitrogen
NaCl sodium chloride
NaHCO₃ sodium bicarbonate
Na₂SO₄ sodium sulfate
NaO^{t}Bu sodium tert-butoxide
NaBH(OAc)₃ sodium triacetoxyborohydride
NMR Nuclear Magnetic Resonance spectroscopy
Pd₂(dba)₃ tris(dibenzylideneacetone)dipalladium (0)
R_{f} retention factor
RT room temperature
Rt retention time
RuPhos 2-dicyclohexylphosphino-2′,6′-diisopropoxybiphenyl
TEA triethylamine
TFA trifluoracetic acid
THF tetrahydrofuran

### Preparation of Imine Prodrugs

Imine prodrugs useful for treating FGF-modulated diseases or injuries are synthesized from commercially available aldehydes 1a-z and commercially available bromide reagents 2a-x or commercially available amine reagents 3a-z using the method shown in Scheme 1. The list of aldehydes 1a-z, bromide reagents 2a-x, and amine reagents 3a-z are provided in Table 1:

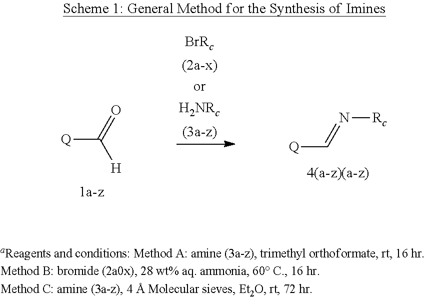

TABLE 1
Aldehydes (1a-z), Bromides (2a-x), Amines (3a-z)

| # | Q | # | R_{c} |
|---|---|---|---|
| 1a | 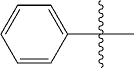 [SMILES: *c1ccccc1] | 2a, 3a | 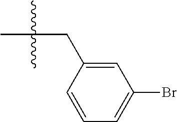 [SMILES: *Cc1cccc(Br)c1] |
| 1b | 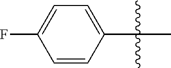 [SMILES: *c1ccc(F)cc1] | 2b, 3b | 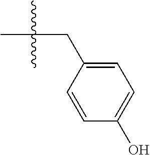 [SMILES: *Cc1ccc(O)cc1] |
| 1c | 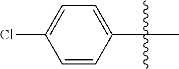 [SMILES: *c1ccc(Cl)cc1] | 2c, 3c | 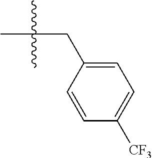 [SMILES: *Cc1ccc(C(F)(F)F)cc1] |
| 1d | 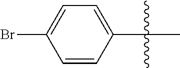 [SMILES: *c1ccc(Br)cc1] | 2d, 3d | 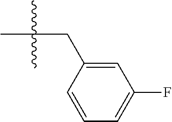 [SMILES: *Cc1cccc(F)c1] |
| 1e | 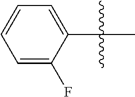 [SMILES: *c1ccccc1F] | 2e, 3e | 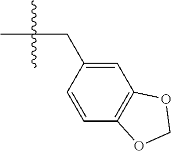 [SMILES: *Cc1ccc2c(c1)OCO2] |
| 1f | 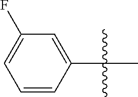 [SMILES: *c1cccc(F)c1] | 2f, 3f | 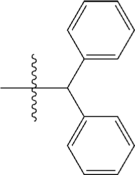 [SMILES: *C(c1ccccc1)c1ccccc1] |
| 1g | 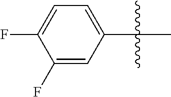 [SMILES: *c1ccc(F)c(F)c1] | 2g, 3g | 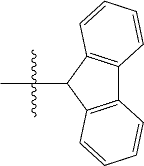 [SMILES: *C1c2ccccc2-c2ccccc21] |
| 1h | 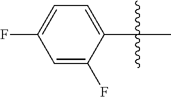 [SMILES: *c1ccc(F)cc1F] | 2h, 3h | 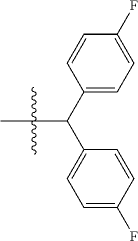 [SMILES: *C(c1ccc(F)cc1)c1ccc(F)cc1] |
| 1i | 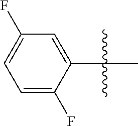 [SMILES: *c1cc(F)ccc1F] | 2i, 3i | 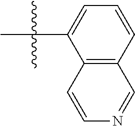 [SMILES: *c1cccc2cnccc12] |
| 1j | 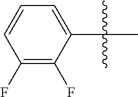 [SMILES: *c1cccc(F)c1F] | 2j, 3j | 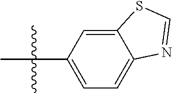 [SMILES: *c1ccc2ncsc2c1] |
| 1k | 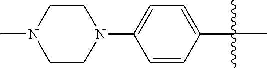 [SMILES: *c1ccc(N2CCN(C)CC2)cc1] | 2k, 3k | 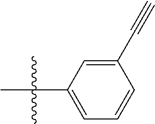 [SMILES: *c1cccc(C#C)c1] |
| 1l | 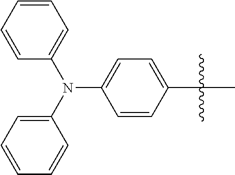 [SMILES: *c1ccc(N(c2ccccc2)c2ccccc2)cc1] | 2l, 3l | 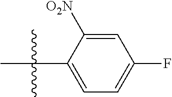 [SMILES: *c1ccc(F)cc1[N+](=O)[O-]] |
| 1m | 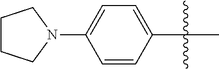 [SMILES: *c1ccc(N2CCCC2)cc1] | 2m, 3m | 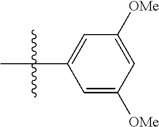 [SMILES: *c1cc(OC)cc(OC)c1] |
| 1n | 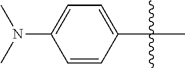 [SMILES: *c1ccc(N(C)C)cc1] | 2n, 3n | 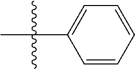 [SMILES: *c1ccccc1] |
| 1o | 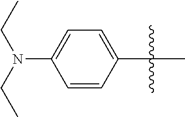 [SMILES: *c1ccc(N(CC)CC)cc1] | 2o, 3o | 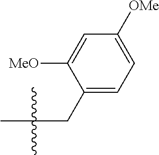 [SMILES: *Cc1ccc(OC)cc1OC] |
| 1p | 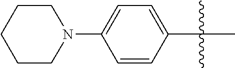 [SMILES: *c1ccc(N2CCCCC2)cc1] | 2p, 3p | 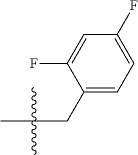 [SMILES: *Cc1ccc(F)cc1F] |
| 1q | 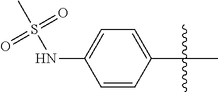 [SMILES: *c1ccc(NS(C)(=O)=O)cc1] | 2q, 3q | 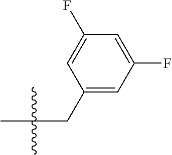 [SMILES: *Cc1cc(F)cc(F)c1] |
| 1r | 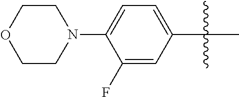 [SMILES: *c1ccc(N2CCOCC2)c(F)c1] | 2r, 3r | 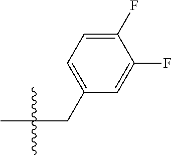 [SMILES: *Cc1ccc(F)c(F)c1] |
| 1s | 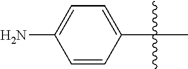 [SMILES: *c1ccc(N)cc1] | 2s, 3s | 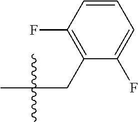 [SMILES: *Cc1c(F)cccc1F] |
| 1t | 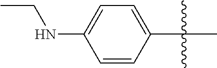 [SMILES: *c1ccc(NCC)cc1] | 2t, 3t | 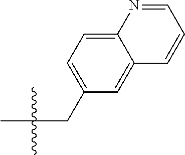 [SMILES: *Cc1ccc2ncccc2c1] |
| 1u | 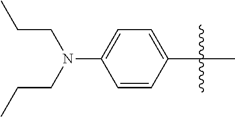 [SMILES: *c1ccc(N(CCC)CCC)cc1] | 2u, 3u | 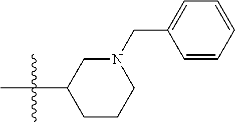 [SMILES: *C1CCCN(Cc2ccccc2)C1] |
| 1v | 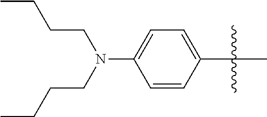 [SMILES: *c1ccc(N(CCCC)CCCC)cc1] | 2v, 3v | 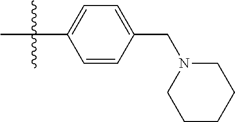 [SMILES: *c1ccc(CN2CCCCC2)cc1] |
| 1w | 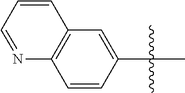 [SMILES: *c1ccc2ncccc2c1] | 2w, 3w | 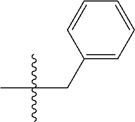 [SMILES: *Cc1ccccc1] |
| 1x | 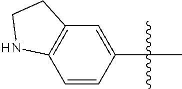 [SMILES: *c1ccc2c(c1)CCN2] | 2x, 3x | 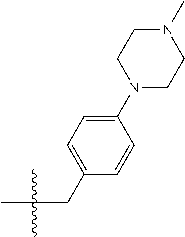 [SMILES: *Cc1ccc(N2CCN(C)CC2)cc1] |
| 1y | 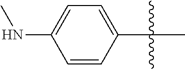 [SMILES: *c1ccc(NC)cc1] | 3y | 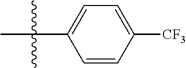 [SMILES: *c1ccc(C(F)(F)F)cc1] |
| 1z | 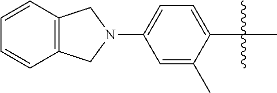 [SMILES: *c1ccc(N2Cc3ccccc3C2)cc1C] | 3z | 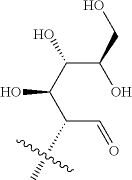 |

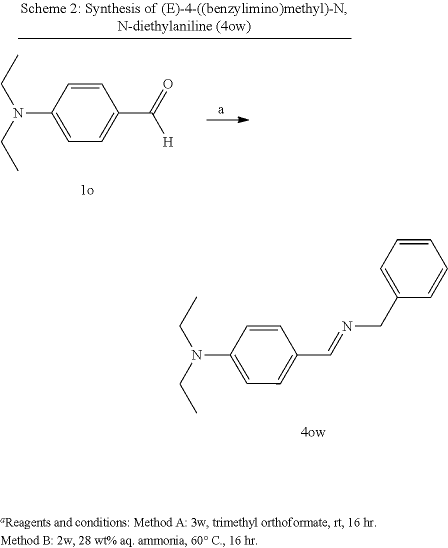 [SMILES: CCN(CC)c1ccc(C=O)cc1 ; CCN(CC)c1ccc(/C=N/Cc2ccccc2)cc1]

### Method A: Preparation of (E)-4-((benzylimino)methyl)-N,N-diethylaniline, Compound 4ow)

To a mixture of 4-diethylaminobenzaldehyde, 10 (Alfa Aesar, 2.01g, 11.3 mmol) and trimethyl orthoformate (Aldrich, 20 mL, 183 mmol) was added benzylamine, 3w (Oakwood, 1.20 g, 11.0 mmol) by dropwise addition. The reaction mixture was stirred at room temperature for 18 hours under N₂ atmosphere. The reaction mixture was then diluted with dichloromethane (300 mL) and the solution was washed with saturated aqueous sodium bicarbonate (2×150 mL). The organic layer was then washed with brine (150 mL), dried over sodium sulfate, and filtered. The filtrate was subsequently concentrated under reduced pressure to obtain a crude yellow oil. The crude yellow oil was purified by flash silica column chromatography on a CombiFlash NextGen 300+ purification system. A 120 g RediSep Gold R_{f} column was conditioned by eluting with 2% TEA/petroleum ether over 3 column volumes. Elution occurred with 1% TEA/ethyl acetate (Solvent A) and heptane using a gradient of 0-20% (Solvent A) over 7 column volumes. After collecting appropriate fractions from the column, the combined fractions were concentrated to obtain the title compound as a clear yellow oil (290 mg, 1.1 mmol, 10% yield); R_{f} 0.65 with TEA:MeOH(1:9)/DCM (7:93) (UV. 254 nM); ¹H-NMR (400 MHZ; CDCl₃) δ 8.15 (s, 1H), 7.59 (d, 2H, J=9.0 Hz), 7.26-7.21 (m, 2H), 7.20-7.15 (m, 1H).), 6.60 (d, 2H, J=9.0 Hz), 4.70 (d, 2H, J=1.2 Hz), 3.33 (q, 4H, J=7.0 Hz), 1.12 (t, 6H, J=7.0 Hz); MS (ES⁺) m/z 267.3 (M+1).

### Method B: Preparation of (E)-4-((benzylimino)methyl)-N,N-diethylaniline, Compound 4ow)

To a sealed tube containing 4-diethylaminobenzaldehyde, 10 (Alfa Aesar, 1.74g, 9.8 mmol) and benzyl bromide, 2w (Oakwood, 2.51 g, 14.7 mmol) was added 20 mL of 28 wt % aqueous ammonia. The reaction mixture was stirred at 60 ° ° C.overnight under N₂ atmosphere. The crude reaction was then extracted with diethyl ether (2×50 mL). The combined organic extracts were dried over anhydrous sodium sulfate, filtered, and the filtrate concentrated under reduced pressure. The crude residue thus obtained was purified by flash silica column chromatography on a CombiFlash NextGen 300+ purification system. A 120g RediSep Gold R_{f} column was conditioned by eluting with 2% TEA/petroleum ether over 3 column volumes. Elution occurred with 1% TEA/ethyl acetate (Solvent A) and heptane using a gradient of 0-20% (Solvent A) over 7 column volumes. After collecting appropriate fractions from the column the combined fractions were concentrated to obtain the title compound as a clear yellow oil (28 mg, 1.2% yield); R_{f} 0.65 with TEA:MeOH(1:9)/DCM (7:93) (UV 254 nM); ¹H-NMR (400 MHZ; CDCl₃) δ 8.15 (s, 1H), 7.59 (d, 2H, J=9.0 Hz), 7.26-7.21 (m, 2H), 7.20-7.15 (m, 1H).), 6.60 (d, 2H, J=9.0 Hz), 4.70 (d, 2H, J=1.2 Hz), 3.33 (q, 4H, J=7.0 Hz), 1.12 (t, 6H, J=7.0 Hz); MS (ES⁺) m/z 267.3 (M+1).

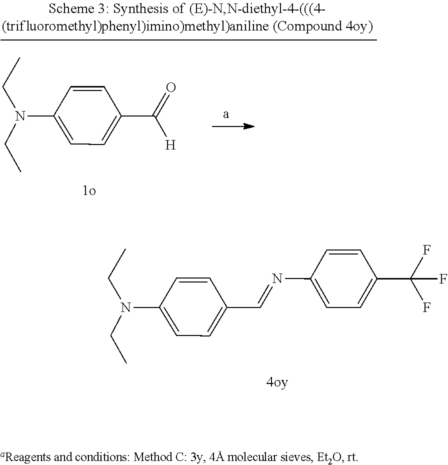 [SMILES: CCN(CC)c1ccc(C=O)cc1 ; CCN(CC)c1ccc(/C=N/c2ccc(C(F)(F)F)cc2)cc1]

### Method C:Preparation of (E)-N, N-diethyl-4-(((4-(trifluoromethyl)phenyl)imino)methyl)aniline, (Compound 4oy)

To a mixture of 4-diethylaminobenzaldehyde, 10 (Alfa Aesar, 0.55g, 3.10 mmol), 4-aminobenzotrifluoride, 3y (Combi-Blocks, 0.50g, 3.10 mmol), and 4 Å molecular sieves was added anhydrous diethyl ether (75 mL). The reaction mixture was stirred at room temperature for 72 hours under N₂ atmosphere. The reaction mixture was subsequently concentrated under reduced pressure to obtain a crude yellow oil. The crude yellow oil was purified by flash silica column chromatography on a CombiFlash NextGen 300+ purification system. A 40 g RediSep Gold R_{f} column was conditioned by eluting with 2% TEA/petroleum ether over 3 column volumes. Elution occurred with ethyl acetate (Solvent A) and heptane using a gradient of 15-40% (Solvent A) over 12 column volumes. After collecting appropriate fractions from the column, the combined fractions were concentrated to obtain the title compound as a clear yellow oil (130 mg, 0.41 mmol, 13% yield); R_{f} 0.80 with EA/Hept (25:75) (UV 254 nM); ¹H-NMR (400 MHZ; CDCl₃) δ 8.35 (s, 1H), 7.69 (br d, 2H, J=9.2 Hz), 7.66 (br d, 2H, J=8.7 Hz), 7.29 (d, 2H, J=8.3 Hz), 6.72 (d, 2H, J=9.2 Hz), 3.39 (q, 4H, J=7.2 Hz), 1.09 (t, 6H, J=6.9 Hz); MS (APCI⁺) m/z 321.1 (M+1); melting point =129.3-129.6 ° C.

### Preparation of Oxime Prodrugs

Oxime prodrugs 5a-z useful for treating FGF-modulated diseases or injuries are synthesized from aldehydes 1a-z according to the general procedure described below (Scheme 4).

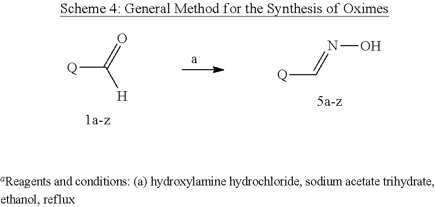

To a solution of aryl aldehyde 1a-z (1 molar equivalents) in a mixture of ethanol and water (10:1) is added hydroxylamine hydrochloride (2 molar equivalents), followed by addition of sodium acetate trihydrate (2 molar equivalents). The reaction mixture is stirred at room temperature under nitrogen atmosphere for 16 hours. After reaction completion the crude reaction mixture is concentrated under reduced pressure to afford a crude residue. The crude residue is dissolved in ethyl acetate and washed with water1. The organic layer is dried over anhydrous sodium sulfate, filtered, and the filtrate concentrated under reduced pressure to affords the desired aryl oxime 5a-z (Table 2). If needed, the crude product is purified by flash silica column chromatography on a CombiFlash NextGen 300+ purification system.

TABLE 2
Oximes

| # | Structure |
|---|---|
| 5a | 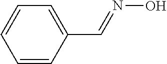 |
| 5b | 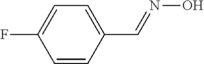 |
| 5c | 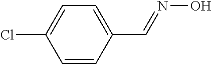 |
| 5d | 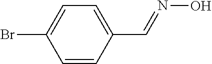 |
| 5e | 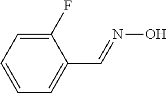 |
| 5f | 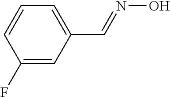 |
| 5g | 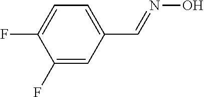 [SMILES: O/N=C/c1ccc(F)c(F)c1] |
| 5h | 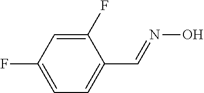 [SMILES: O/N=C/c1ccc(F)cc1F] |
| 5i | 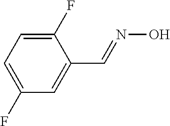 [SMILES: O/N=C/c1cc(F)ccc1F] |
| 5j | 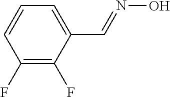 [SMILES: O/N=C/c1cccc(F)c1F] |
| 5k | 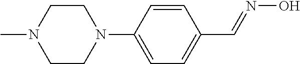 [SMILES: CN1CCN(c2ccc(/C=N/O)cc2)CC1] |
| 5l | 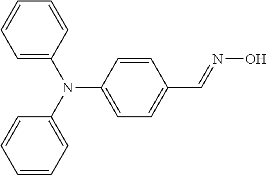 [SMILES: O/N=C/c1ccc(N(c2ccccc2)c2ccccc2)cc1] |
| 5m | 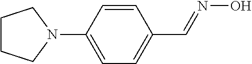 [SMILES: O/N=C/c1ccc(N2CCCC2)cc1] |
| 5n | 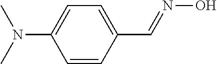 |
| 5o | 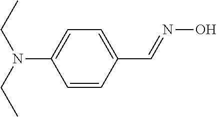 |
| 5p | 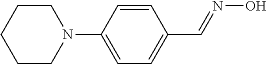 [SMILES: O/N=C/c1ccc(N2CCCCC2)cc1] |
| 5q | 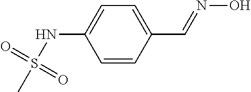 |
| 5r | 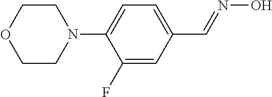 [SMILES: O/N=C/c1ccc(N2CCOCC2)c(F)c1] |
| 5s | 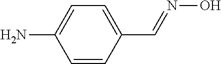 |
| 5t | 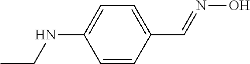 |
| 5u | 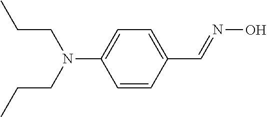 [SMILES: CCCN(CCC)c1ccc(/C=N/O)cc1] |
| 5v | 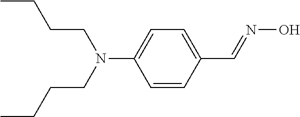 [SMILES: CCCCN(CCCC)c1ccc(/C=N/O)cc1] |
| 5w | 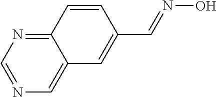 [SMILES: O/N=C/c1ccc2ncncc2c1] |
| 5x | 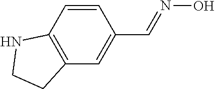 [SMILES: O/N=C/c1ccc2c(c1)CCN2] |
| 5y | 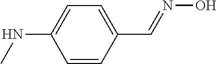 |
| 5z | 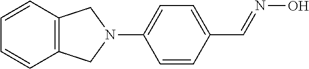 [SMILES: O/N=C/c1ccc(N2Cc3ccccc3C2)cc1] |

### Preparation of Hydrazine Prodrugs

Hydrazine prodrugs 6a-z useful for treating FGF-modulated diseases or injuries are synthesized from oximes 5a-z according to the general procedure described below (Scheme 5).

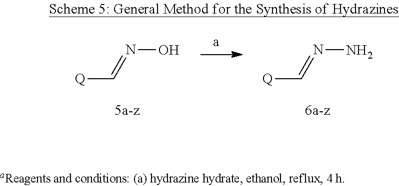

To a solution of oxime 5a-z (1 molar equivalent) in ethanol is added 99-100% hydrazine hydrate. The reaction mixture is refluxed under N₂ atmosphere for 4 hours. After reaction shows completion by disappearance of the staring material on TLC, the crude reaction mixture is diluted with water and extracted with ether. Concentration of the organic layer under reduced pressure affords hydrazine 6a-z (Table 3). If needed, the crude product is purified by flash silica column chromatography on a CombiFlash NextGen 300+ purification system.

TABLE 3
Hydrazines

| # | Structure |
|---|---|
| 6a | 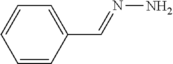 |
| 6b | 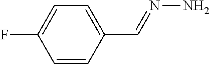 |
| 6c | 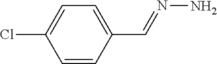 |
| 6d | 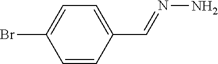 |
| 6e | 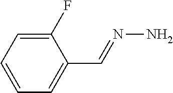 |
| 6f | 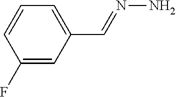 |
| 6g | 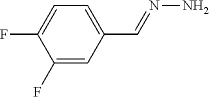 [SMILES: N/N=C/c1ccc(F)c(F)c1] |
| 6h | 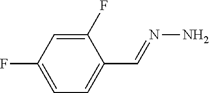 [SMILES: N/N=C/c1ccc(F)cc1F] |
| 6i | 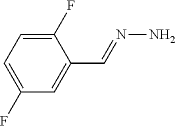 [SMILES: N/N=C/c1cc(F)ccc1F] |
| 6j | 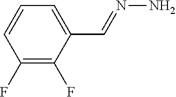 [SMILES: N/N=C/c1cccc(F)c1F] |
| 6k | 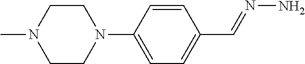 [SMILES: CN1CCN(c2ccc(/C=N/N)cc2)CC1] |
| 6l | 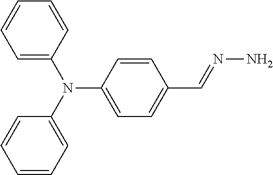 [SMILES: N/N=C/c1ccc(N(c2ccccc2)c2ccccc2)cc1] |
| 6m | 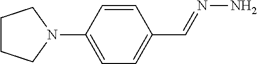 [SMILES: N/N=C/c1ccc(N2CCCC2)cc1] |
| 6n | 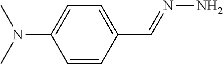 |
| 6o | 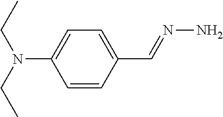 |
| 6p | 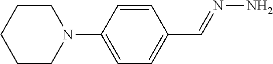 [SMILES: N/N=C/c1ccc(N2CCCCC2)cc1] |
| 6q | 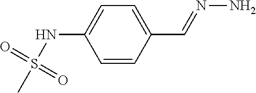 |
| 6r | 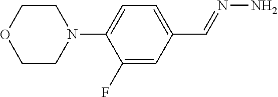 [SMILES: N/N=C/c1ccc(N2CCOCC2)c(F)c1] |
| 6s | 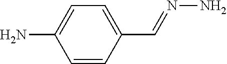 |
| 6t | 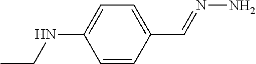 |
| 6u | 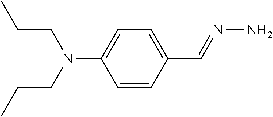 [SMILES: CCCN(CCC)c1ccc(/C=N/N)cc1] |
| 6v | 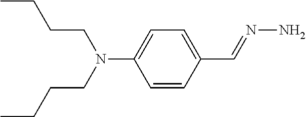 [SMILES: CCCCN(CCCC)c1ccc(/C=N/N)cc1] |
| 6w | 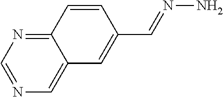 [SMILES: N/N=C/c1ccc2ncncc2c1] |
| 6x | 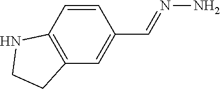 [SMILES: N/N=C/c1ccc2c(c1)CCN2] |
| 6y | 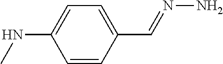 |
| 6z | 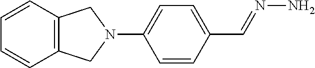 [SMILES: N/N=C/c1ccc(N2Cc3ccccc3C2)cc1] |

### Preparation of Benzophenone Prodrugs

Benzophenone prodrugs useful for treating FGF-modulated diseases or injuries are synthesized from commercially available aldehydes 1a-z and commercially available iodide reagents 7a-x using the method shown in Scheme 6. The list of aldehydes 1a-z are provided in Table 1. The aryl iodide reagents 7a-p are provided in Table 4:

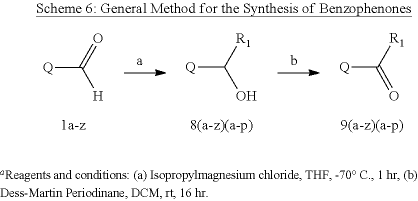

To a solution of aryl iodide 7a-p (1 molar equivalent) in THF is added isopropyl magnesium chloride (2 M solution in THF, 1.3 molar equivalents) at −78° C. The reaction mixture is stirred under N₂ atmosphere and allowed to warm to 0° C. over one hour. Next the reaction mixture is cooled back to −78° C. and 3a-z (1 molar equivalents) is added dropwise as a solution in THF. The reaction mixture is stirred overnight and warmed to room temperature under N₂ atmosphere. Upon completion, the reaction mixture is quenched with aqueous saturated ammonium chloride solution. The reaction mixture is portioned in a separatory funnel and the organic layer is extracted with MTBE. The combined organic layer is dried over anhydrous sodium sulfate, filtered, and concentrated under reduced pressure to give the crude product. The crude product is purified by flash silica column chromatography to afford the alcohol 8(a-z)(a-p).

A solution of alcohol 8(a-z)(a-p) (1 molar equivalent) and Dess-Martin Periodinane (1.2 molar equivalents) in dichloromethane is stirred overnight at room temperature under N₂ atmosphere. Upon completion, the reaction mixture is quenched with aqueous NaOH. The reaction mixture is portioned in a separatory funnel and the organic layer is extracted with dichloromethane and ethyl acetate. The combined organic layer is dried over anhydrous sodium sulfate, filtered, and concentrated under reduced pressure to give the crude product. The crude product is purified by flash silica column chromatography to afford the title compound.

TABLE 4
Aryl Iodides

| # | Structure |
|---|---|
| 7a | 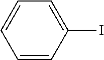 |
| 7b | 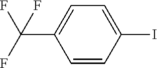 [SMILES: FC(F)(F)c1ccc(I)cc1] |
| 7c | 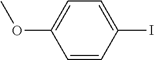 |
| 7d | 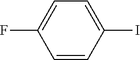 |
| 7e | 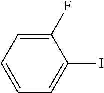 |
| 7f | 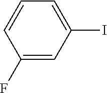 |
| 7g | 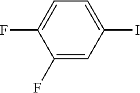 |
| 7h | 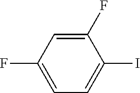 |
| 7i | 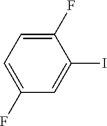 |
| 7j | 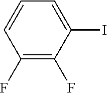 |
| 7k | 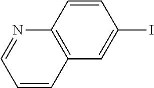 [SMILES: Ic1ccc2ncccc2c1] |
| 7l | 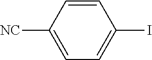 |
| 7m | 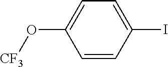 [SMILES: FC(F)(F)Oc1ccc(I)cc1] |
| 7n | 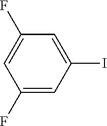 |
| 7o | 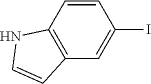 |
| 7p | 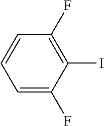 |

### Preparation of (4-(diethylamino)phenyl)(3-(trifluoromethyl)phenyl)methanone (Compound 9ob)

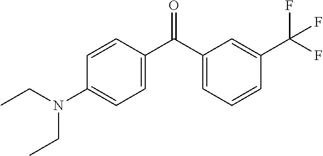 [SMILES: CCN(CC)c1ccc(C(=O)c2cccc(C(F)(F)F)c2)cc1]

### Step a: Preparation of (4-(diethylamino)phenyl)(3-(trifluoromethyl)phenyl)methanol (Compound 80b)

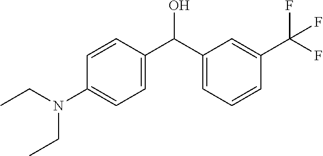 [SMILES: CCN(CC)c1ccc(C(O)c2cccc(C(F)(F)F)c2)cc1]

To a solution of 4-iodobentrifluoride 7b (Combi-Blocks, 1g, 3.67 mmol) in THF (50 mL) was added isopropyl magnesium chloride (Aldrich, 2M solution in THF, 2.39 mL, 4.78 mmol) at −78° C. The reaction mixture was stirred under N₂ atmosphere and allowed to warm to 0° C. over one hour. Next the reaction mixture was cooled back to −78° C. and 4-diethylaminobenzaldhyde 10 (Alfa Aesar, 0.65 g, 3.67 mmol) was added dropwise as a solution in THF (5 mL). The reaction mixture was stirred overnight warming to room temperature under N₂ atmosphere. Upon completion, the reaction mixture was quenched with aqueous saturated ammonium chloride solution. The reaction mixture was portioned in a separatory funnel and the organic layer was extracted with MTBE (2×50 mL). The combined organic layer was dried over anhydrous sodium sulfate, filtered, and concentrated under reduced pressure to give the crude product. The crude solid was purified by flash silica column chromatography on a CombiFlash NextGen 300+ purification system. Elution through a 40 g RediSep Gold R_{f} flash silica cartridge with 0-50% ethyl acetate in hexanes afforded the title compound as a yellow oil (0.94 g, 79%); R_{f} 0.25 with 75:25 v/v hexanes-ethyl acetate (UV. 254 nM); MS (ES⁺) m/z 322.1 (M+1).

### Step b: Preparation of (4-(diethylamino)phenyl)(3-(trifluoromethyl)phenyl)methanone (Compound 9ob)

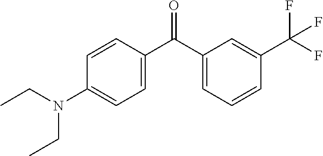 [SMILES: CCN(CC)c1ccc(C(=O)c2cccc(C(F)(F)F)c2)cc1]

A solution of alcohol 8ob (0.94 g, 2.94 mmol) and Dess-Martin Periodinane (1.49 g, 3.52 mmol) in dichloromethane (50 mL) is stirred overnight at room temperature under N₂ atmosphere. Upon completion, the reaction mixture was quenched with aqueous NaOH. The reaction mixture was portioned in a separatory funnel and the organic layer was extracted with dichloromethane and ethyl acetate. The combined organic layer was dried over anhydrous sodium sulfate, filtered, and concentrated under reduced pressure to give the crude product. The crude product was purified by flash silica column chromatography on a CombiFlash NextGen 300+ purification system. A 40 g RediSep Gold R_{f} column was pre-conditioned by eluting with 1% TEA/Heptane over 3 column volumes. Elution occurred with ethyl acetate/TEA (1%) (Solvent A) and heptane/TEA (1%) using a gradient of 5-25% (Solvent A) over 15 column volumes. After collecting appropriate fractions from the column, the combined fractions were concentrated to obtain the title compound as a clear yellow oil which solidified upon standing (101 mg, 0.31 mmol, 11% yield); R_{f} 0.60 with EA/Hept (25:75) (UV. 254 nM); ¹H-NMR (400 MHZ; DMSO-d₆) δ 7.84 (d, 2H, J=8.3 Hz), 7.76 (d, 2H, J=8.3 Hz), 7.58 (d, 2H, J=7.9 Hz), 6.70 (d, 2H, J=8.0 Hz), 3.40 (q, 4H, J=6.9 Hz), 1.09 (t, 6H, J=7.1 Hz); MS (APCI⁺) m/z 322.2 (M+1); HPLC UV purity, Rt =19.79 min, 96.88%; melting point =63.1-63.3° C.

### Preparation of (4-(diethylamino)phenyl)(3-methoxyphenyl)methanone (Compound 9oc)

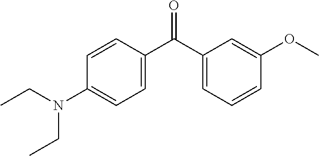 [SMILES: CCN(CC)c1ccc(C(=O)c2cccc(OC)c2)cc1]

### Step a: Preparation of (4-(diethylamino)phenyl)(3-methoxyphenyl)methanol (Compound 8oc)

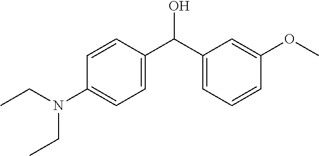 [SMILES: CCN(CC)c1ccc(C(O)c2cccc(OC)c2)cc1]

To a solution of 4-iodoanisole, 7c (Combi-Blocks, 1g, 4.27 mmol) in THF (50 mL) was added isopropyl magnesium chloride (Aldrich, 2M solution in THF, 2.78 mL, 5.56 mmol) at −78° C. The reaction mixture was stirred under N₂ atmosphere and allowed to warm to 0 ° C. over one hour. Next the reaction mixture was cooled back to −78° C. and 4-diethylaminobenzaldhyde 10 (Alfa Aesar, 0.76 g, 4.27 mmol) is added dropwise as a solution in THF (5 mL). The reaction mixture was stirred for 72 hours warming to room temperature under N₂ atmosphere. Upon completion, the reaction mixture was quenched with aqueous saturated ammonium chloride solution. The reaction mixture is portioned in a separatory funnel and the organic layer is extracted with MTBE (2×50 mL). The combined organic layer is dried over anhydrous sodium sulfate, filtered, and concentrated under reduced pressure to give the crude product. The crude solid was purified by flash silica column chromatography on a CombiFlash NextGen 300+ purification system. Elution occurred through a 40 g RediSep Gold R_{f} flash silica cartridge with 10-50% ethyl acetate in hexanes over 15 column volumes. After collecting appropriate fractions from the column, the combined fractions were concentrated to obtain the title compound as a clear yellow oil which solidified upon standing afforded the title compound as a yellow oil (0.73 g, 60%); R_{f} 0.20 with 75:25 v/v hexanes-ethyl acetate (UV. 254 nM); MS (ES⁺) m/z 286.4 (M+1)

### Step b: Preparation of (4-(diethylamino)phenyl)(3-methoxyphenyl)methanone (Compound 9oc)

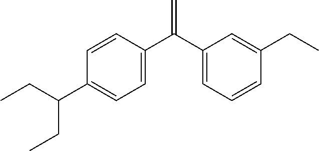 [SMILES: C=C(c1ccc(C(CC)CC)cc1)c1cccc(CC)c1]

A solution of alcohol 8oc (0.73 g, 2.57 mmol) and Dess-Martin Periodinane (1.31 g, 3.08 mmol) in dichloromethane (50 mL) was stirred overnight at room temperature under N₂ atmosphere. Upon completion, the reaction mixture was quenched with aqueous NaOH. The reaction mixture was portioned in a separatory funnel and the organic layer was extracted with dichloromethane and ethyl acetate. The combined organic layer was dried over anhydrous sodium sulfate, filtered, and concentrated under reduced pressure to give the crude product. The crude product was purified by flash silica column chromatography on a CombiFlash NextGen 300+ purification system. A 40 g RediSep Gold R_{f} column was pre-conditioned by eluting with 1% TEA/Heptane over 3 column volumes. Elution occurred with ethyl acetate/TEA (1%) (Solvent A) and heptane/TEA (1%) using a gradient of 10-90% (Solvent A) over 15 column volumes. After collecting appropriate fractions from the column, the combined fractions were concentrated to obtain the title compound as a clear green oil which solidified upon standing (35 mg, 0.12 mmol, 5% yield); R_{f} 0.50 with EA/Hept (25:75) (UV. 254 nM); ¹H-NMR (400 MHZ; CDCl₃) δ 7.8-7.9 (m, 1H), 7.7-7.8 (m, 3H), 7.6-7.1 (m, 1H), 6.98 (dd, 3H, J=6.9, 8.7 Hz), 3.6-3.7 (m, 4H), 1.2-1.3 (m, 6H); MS (APCI⁺) m/z 284.3 (M+1); HPLC UV purity, Rt =19.79 min, 96.88%; melting point =87.6-88.7° C.

### Preparation of (4-(diethylamino)phenyl)(3-(trifluoromethoxy)phenyl)methanone methanone (Compound 9om)

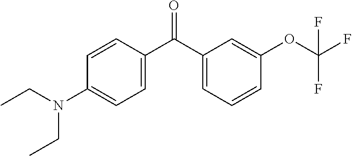 [SMILES: CCN(CC)c1ccc(C(=O)c2cccc(OC(F)(F)F)c2)cc1]

### Step a: Preparation of (4-(diethylamino)phenyl)(3-(trifluoromethoxy)phenyl)methano (Compound 8om)

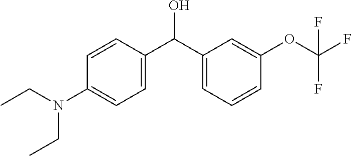 [SMILES: CCN(CC)c1ccc(C(O)c2cccc(OC(F)(F)F)c2)cc1]

To a solution of 1-iodo-4-(trifluoromethoxy)benzene 7m (Combi-Blocks, 1g, 3.47 mmol) in THF (50 mL) was added isopropyl magnesium chloride (Aldrich, 2M solution in THF, 2.39 mL, 4.78 mmol) at -78° C. The reaction mixture was stirred under N₂ atmosphere and allowed to warm to 0 ° C.over one hour. Next the reaction mixture was cooled back to -78° C. and 4-diethylaminobenzaldhyde 10 (Alfa Aesar, 0.62 g, 3.47 mmol) was added dropwise as a solution in THF (5 mL). The reaction mixture was stirred overnight warming to room temperature under N₂ atmosphere. Upon completion, the reaction mixture was quenched with aqueous saturated ammonium chloride solution. The reaction mixture was portioned in a separatory funnel and the organic layer was extracted with MTBE (2×50 mL). The combined organic layer was dried over anhydrous sodium sulfate, filtered, and concentrated under reduced pressure to give the crude product. The crude oil was purified by flash silica column chromatography on a CombiFlash NextGen 300+ purification system. Elution through a 40 g RediSep Gold R_{f} flash silica cartridge with 0-50% ethyl acetate in hexanes afforded the title compound as an orange oil (0.41 g, 35% yield); R_{f} 0.25 with 75:25 v/v hexanes-ethyl acetate (UV. 254 nM);); ¹H-NMR (400 MHZ; DMSO-d₆) δ 7.45 (d, 2H, J=8.0 Hz), 7.27 (d, 2H, J=7.8 Hz), 7.10 (d, 2H, J=8.7 Hz), 6.58 (d, 2H, J=9.2 Hz), 5.71 (d, 1H, J=3.7 Hz), 5.59 (d, 1H, J=3.7 Hz), 3.28 (q, 4H, J=7.1 Hz), 1.04 (t, 6H, J=7.1 Hz); MS (ES⁺) m/z 340.3 (M+1); HPLC UV purity, Rt =7.365 min, 98.48%;

### Step b: Preparation of (4-(diethylamino)phenyl)(3-(trifluoromethoxy)phenyl)methanone (Compound 9om)

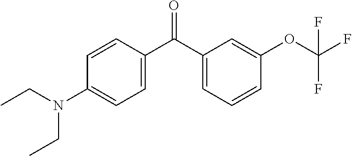 [SMILES: CCN(CC)c1ccc(C(=O)c2cccc(OC(F)(F)F)c2)cc1]

A solution of alcohol 8om (0.41 g, 1.43 mmol) and Dess-Martin Periodinane (1.04 g, 2.45 mmol) in dichloromethane (50 mL) is stirred overnight at room temperature under N₂ atmosphere. Upon completion, the reaction mixture was quenched with aqueous NaOH. The reaction mixture was portioned in a separatory funnel and the organic layer was extracted with dichloromethane and ethyl acetate. The combined organic layer was dried over anhydrous sodium sulfate, filtered, and concentrated under reduced pressure to give the crude product. The crude product was purified by flash silica column chromatography on a CombiFlash NextGen 300+ purification system. Elution through a 12g RediSep Gold R_{f} column with 0-10% ethyl acetate in hexanes afforded the title compound as a clear yellow oil (36 mg, 0.10 mmol, 7.7% yield); ¹H-NMR (400 MHZ; DMSO-d₆) δ 7.84 (d, 2H, J=8.3 Hz), 7.76 (d, 2H, J=8.3 Hz), 7.58 (d, 2H, J=7.9 Hz), 6.70 (d, 2H, J=8.0 Hz), 3.40 (q, 4H, J=6.9 Hz), 1.09 (t, 6H, J=7.1 Hz); MS (APCI⁺) m/z 338.10 (M+1); HPLC UV purity, Rt =7.72 min, 97.98%.

### Preparation of Hydrazine Condensate Prodrugs

Hydrazine condensate prodrugs useful for treating FGF-modulated diseases or injuries are synthesized from commercially available aldehydes 1a-z and commercially hydrazine hydrate using the method shown in Scheme 7. The list of aldehydes 1a-z are provided in Table 1.

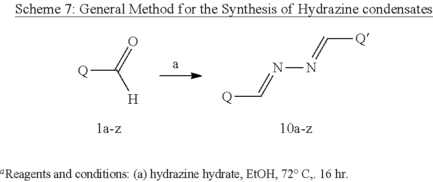

To a solution of 99-100% hydrazine hydrate (1 molar equivalents) in water is added aldehyde 1a-z (2 molar equivalents) as a solution in ethanol. The reaction mixture is heated to 72° C. overnight while under N₂ atmosphere. After reaction shows completion by disappearance of the staring material on TLC, the crude reaction mixture is diluted with water and the precipitated solid is filtered over a fritted funnel which affords the hydrazine condensates 10a-z (Table 5). If needed, the crude product is purified by flash silica column chromatography on a CombiFlash NextGen 300+ purification system.

TABLE 5
Hydrazine condensates

| # | Structure |
|---|---|
| 10a | 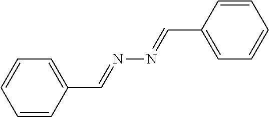 [SMILES: C(=N\N=C\c1ccccc1)/c1ccccc1] |
| 10b | 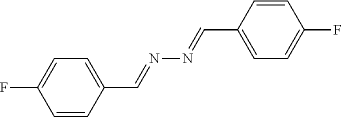 [SMILES: Fc1ccc(/C=N/N=C/c2ccc(F)cc2)cc1] |
| 10c | 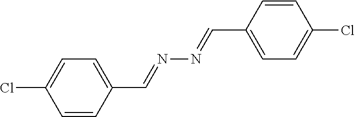 [SMILES: Clc1ccc(/C=N/N=C/c2ccc(Cl)cc2)cc1] |
| 10d | 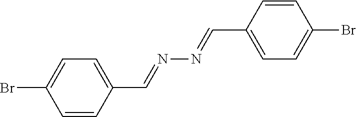 [SMILES: Brc1ccc(/C=N/N=C/c2ccc(Br)cc2)cc1] |
| 10e | 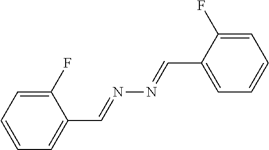 [SMILES: Fc1ccccc1/C=N/N=C/c1ccccc1F] |
| 10f | 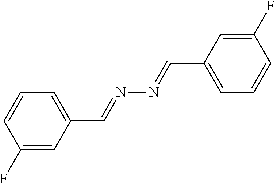 [SMILES: Fc1cccc(/C=N/N=C/c2cccc(F)c2)c1] |
| 10g | 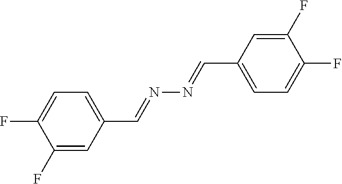 [SMILES: Fc1ccc(/C=N/N=C/c2ccc(F)c(F)c2)cc1F] |
| 10h | 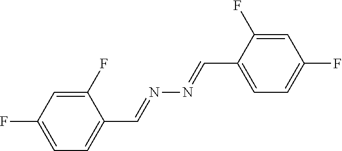 [SMILES: Fc1ccc(/C=N/N=C/c2ccc(F)cc2F)c(F)c1] |
| 10i | 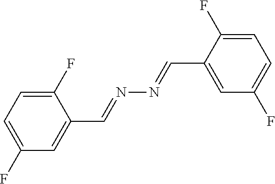 [SMILES: Fc1ccc(F)c(/C=N/N=C/c2cc(F)ccc2F)c1] |
| 10j | 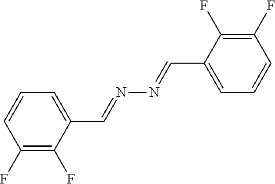 [SMILES: Fc1cccc(/C=N/N=C/c2cccc(F)c2F)c1F] |
| 10k | 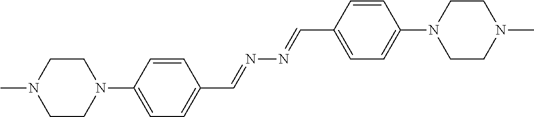 |
| 10l | 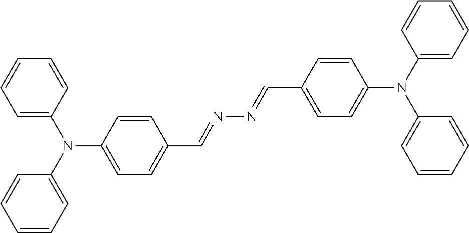 [SMILES: C(=N\N=C\c1ccc(N(c2ccccc2)c2ccccc2)cc1)/c1ccc(N(c2ccccc2)c2ccccc2)cc1] |
| 10m | 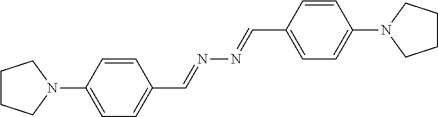 [SMILES: C(=N\N=C\c1ccc(N2CCCC2)cc1)/c1ccc(N2CCCC2)cc1] |
| 10n | 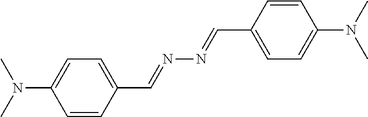 |
| 10o | 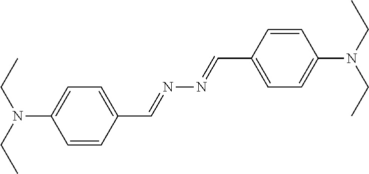 [SMILES: CCN(CC)c1ccc(/C=N/N=C/c2ccc(N(CC)CC)cc2)cc1] |
| 10p | 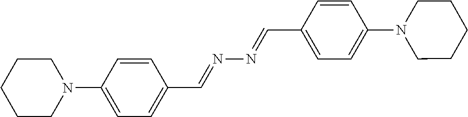 [SMILES: C(=N\N=C\c1ccc(N2CCCCC2)cc1)/c1ccc(N2CCCCC2)cc1] |
| 10q | 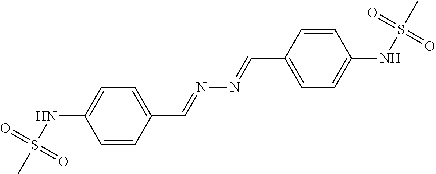 [SMILES: CS(=O)(=O)Nc1ccc(/C=N/N=C/c2ccc(NS(C)(=O)=O)cc2)cc1] |
| 10r | 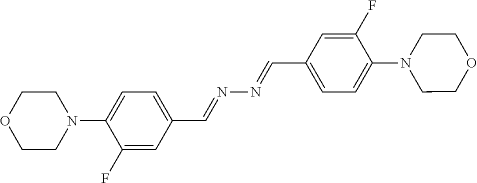 [SMILES: Fc1cc(/C=N/N=C/c2ccc(N3CCOCC3)c(F)c2)ccc1N1CCOCC1] |
| 10s | 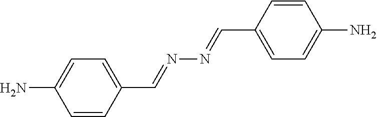 [SMILES: Nc1ccc(/C=N/N=C/c2ccc(N)cc2)cc1] |
| 10t | 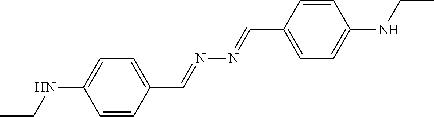 [SMILES: CCNc1ccc(/C=N/N=C/c2ccc(NCC)cc2)cc1] |
| 10u | 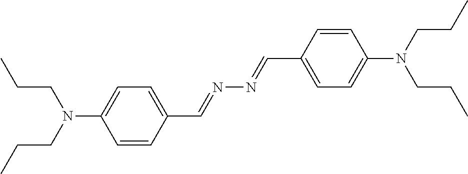 [SMILES: CCCN(CCC)c1ccc(/C=N/N=C/c2ccc(N(CCC)CCC)cc2)cc1] |
| 10v | 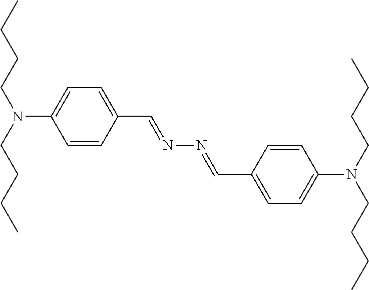 [SMILES: CCCCN(CCCC)c1ccc(/C=N/N=C/c2ccc(N(CCCC)CCCC)cc2)cc1] |
| 10w | 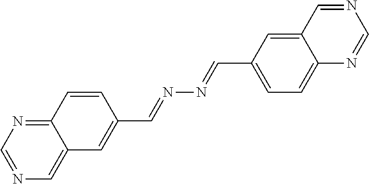 [SMILES: C(=N\N=C\c1ccc2ncncc2c1)/c1ccc2ncncc2c1] |
| 10x | 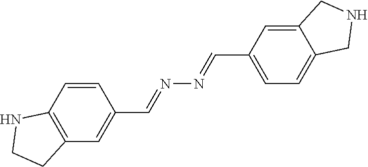 [SMILES: C(=N\N=C\c1ccc2c(c1)CCN2)/c1ccc2c(c1)CNC2] |
| 10y | 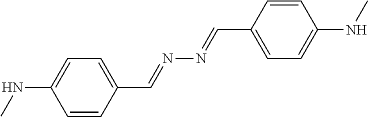 |
| 10z | 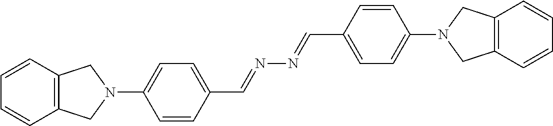 [SMILES: C(=N\N=C\c1ccc(N2Cc3ccccc3C2)cc1)/c1ccc(N2Cc3ccccc3C2)cc1] |

### Preparation of 1,2-bis((E)-3-fluorobenzylidene)hydrazine, (Compound 10f)

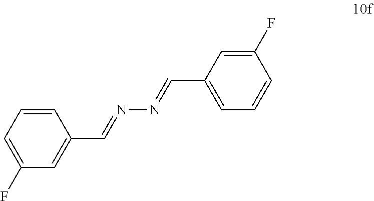 [SMILES: Fc1cccc(/C=N/N=C/c2cccc(F)c2)c1]

To a solution of hydrazine hydrate (Aldrich, 0.057 g, 1.75 mmol) in water (2 mL) was added 3-fluorobenzaldehyde, 1f (Alfa Aesar, 0.440 g, 3.55 mmol). Next ethanol (5 mL) was added and the reaction mixture was stirred at 72 ºC for 16 hours under N₂ atmosphere. After stirring ovemight, a yellow precipitate formed in the solution. Next the reaction mixture was diluted with water (10 mL) and the solution was filtered over a fritted funnel. The filtered solid was washed with water and then dried to obtain the crude compound. The crude product was purified by flash silica column chromatography on a CombiFlash NextGen 300+ purification system. Elution through a 12g RediSep Gold R column with 5-50% ethyl acetate in hexanes afforded the title compound as a yellow crystalline solid (239 mg, 0.98 mmol, 56% yield); R_{f} 0.56 with 85:15 v/v hexanes-ethyl acetate (UV 254 nM); ¹H-NMR (400 MHZ; DMSO-d₆) δ 8.73 (s, 2H), 7.7-7.8 (m, 4H), 7.57 (dt, 2H, J=6.0, 8.7 Hz), 7.39 (t, 2H, J=8.7 Hz); MS (ES⁺) m/z 245.10 (M+1); HPLC UV purity, Rt =7.442 min, 98.57%; melting point =137-139° C.

### Preparation of 4,4′-((1E,1′E)-hydrazine-1,2-diylidenebis(methaneylylidene))bis(N,N-diethylaniline), (Compound 100)

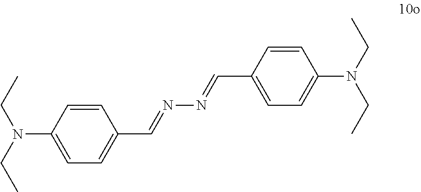 [SMILES: CCN(CC)c1ccc(/C=N/N=C/c2ccc(N(CC)CC)cc2)cc1]

To a solution of hydrazine hydrate (Aldrich, 0.135 g, 1.75 mmol) in water (2 mL) was added 4-diethylaminobenzaldehyde, 10 (Alfa Aesar, 0.629 g, 3.55 mmol). Next, ethanol (3 mL) was added and the reaction mixture was stirred at 72 ºC for 16 hours under N₂ atmosphere. After stirring overnight, a yellow precipitate formed in the solution. Next, the reaction mixture was diluted with water (10 mL) and the solution was filtered over a fritted funnel. The filtered solid was washed with water and then dried to obtain the title compound as a yellow solid (475 mg, 1.35 mmol, 77% yield); R_{f} 0.59 with 70:30 v/v hexanes-ethyl acetate (UV 254 nM); ¹H-NMR (400 MHZ; DMSO-d₆) δ 8.46 (s, 2H), 7.60 (d, 4H, J=8.7 Hz), 6.71 (d, 4H, J=9.2 Hz), 3.3-3.4 (m, 8H), 1.12 (t, 12H, J=7.1 Hz); MS (ES⁺) m/z 351.2 (M+1); HPLC UV purity, Rt=19.95 min, 98.04%; melting point=192-194° C.

### Preparation of Thiazolidine Prodrugs

Hydrazine condensate prodrugs useful for treating FGF-modulated diseases or injuries are synthesized from commercially available aldehydes 1a-z and commercially available penicillamine using the method shown in Scheme 8. The list of aldehydes 1a-z are provided in Table 1.

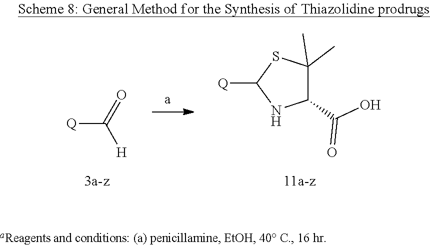

To a solution of aldehyde 1a-z (1 molar equivalent) in ethanol is added penicillamine (1 molar equivalent). The reaction mixture is heated to 40° C. overnight under N₂ atmosphere. After reaction shows completion by disappearance of the staring material on TLC, the crude reaction mixture is diluted with ethanol and the precipitated solid is filtered over a fritted funnel which affords the thiazolidines 11a-z (Table 6). If needed, the crude product is purified by flash silica column chromatography on a CombiFlash NextGen 300+ purification system.

TABLE 6
Thiazolidine prodrugs

| # | Structure |
|---|---|
| 11a | 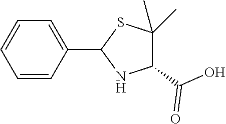 [SMILES: CC1(C)SC(c2ccccc2)N[C@H]1C(=O)O] |
| 11b | 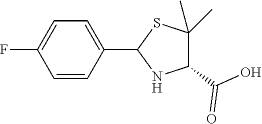 [SMILES: CC1(C)SC(c2ccc(F)cc2)N[C@H]1C(=O)O] |
| 11c | 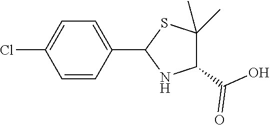 [SMILES: CC1(C)SC(c2ccc(Cl)cc2)N[C@H]1C(=O)O] |
| 11d | 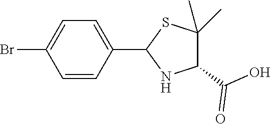 [SMILES: CC1(C)SC(c2ccc(Br)cc2)N[C@H]1C(=O)O] |
| 11e | 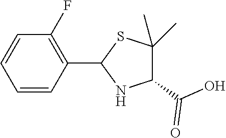 [SMILES: CC1(C)SC(c2ccccc2F)N[C@H]1C(=O)O] |
| 11f | 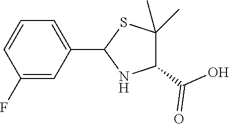 [SMILES: CC1(C)SC(c2cccc(F)c2)N[C@H]1C(=O)O] |
| 11g | 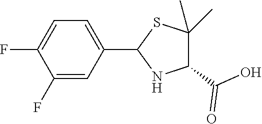 [SMILES: CC1(C)SC(c2ccc(F)c(F)c2)N[C@H]1C(=O)O] |
| 11h | 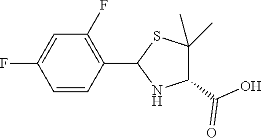 [SMILES: CC1(C)SC(c2ccc(F)cc2F)N[C@H]1C(=O)O] |
| 11i | 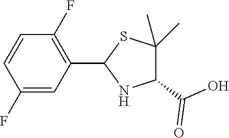 [SMILES: CC1(C)SC(c2cc(F)ccc2F)N[C@H]1C(=O)O] |
| 11j | 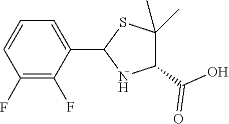 [SMILES: CC1(C)SC(c2cccc(F)c2F)N[C@H]1C(=O)O] |
| 11k | 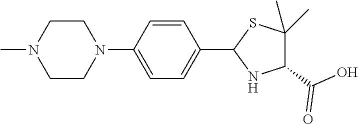 [SMILES: CN1CCN(c2ccc(C3N[C@@H](C(=O)O)C(C)(C)S3)cc2)CC1] |
| 11l | 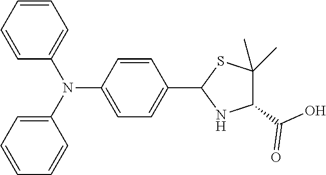 [SMILES: CC1(C)SC(c2ccc(N(c3ccccc3)c3ccccc3)cc2)N[C@H]1C(=O)O] |
| 11m | 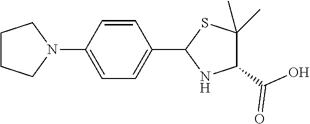 [SMILES: CC1(C)SC(c2ccc(N3CCCC3)cc2)N[C@H]1C(=O)O] |
| 11n | 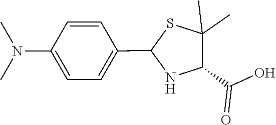 [SMILES: CN(C)c1ccc(C2N[C@@H](C(=O)O)C(C)(C)S2)cc1] |
| 11o | 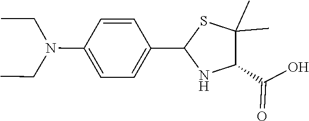 [SMILES: CCN(CC)c1ccc(C2N[C@@H](C(=O)O)C(C)(C)S2)cc1] |
| 11p | 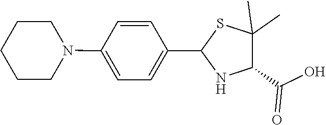 [SMILES: CC1(C)SC(c2ccc(N3CCCCC3)cc2)N[C@H]1C(=O)O] |
| 11q | 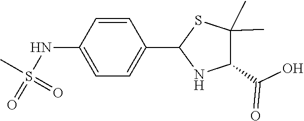 [SMILES: CC1(C)SC(c2ccc(NS(C)(=O)=O)cc2)N[C@H]1C(=O)O] |
| 11r | 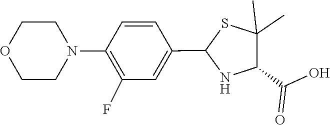 [SMILES: CC1(C)SC(c2ccc(N3CCOCC3)c(F)c2)N[C@H]1C(=O)O] |
| 11s | 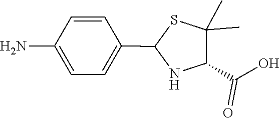 [SMILES: CC1(C)SC(c2ccc(N)cc2)N[C@H]1C(=O)O] |
| 11t | 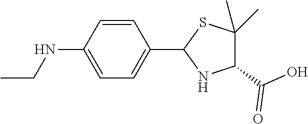 [SMILES: CCNc1ccc(C2N[C@@H](C(=O)O)C(C)(C)S2)cc1] |
| 11u | 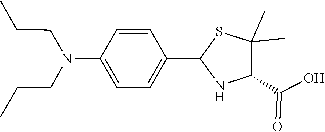 [SMILES: CCCN(CCC)c1ccc(C2N[C@@H](C(=O)O)C(C)(C)S2)cc1] |
| 11v | 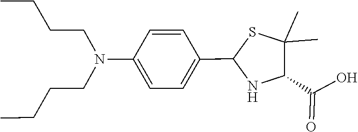 [SMILES: CCCCN(CCCC)c1ccc(C2N[C@@H](C(=O)O)C(C)(C)S2)cc1] |
| 11w | 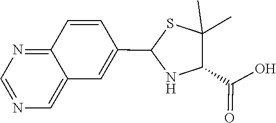 [SMILES: CC1(C)SC(c2ccc3ncncc3c2)N[C@H]1C(=O)O] |
| 11x | 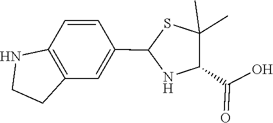 [SMILES: CC1(C)SC(c2ccc3c(c2)CCN3)N[C@H]1C(=O)O] |
| 11y | 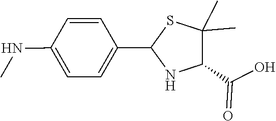 [SMILES: CNc1ccc(C2N[C@@H](C(=O)O)C(C)(C)S2)cc1] |
| 11z | 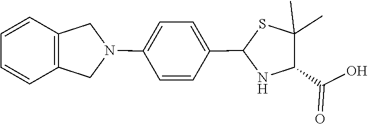 [SMILES: CC1(C)SC(c2ccc(N3Cc4ccccc4C3)cc2)N[C@H]1C(=O)O] |

### Preparation of (4S)-2-(4-(diethylamino)phenyl)-5,5-dimethylthiazolidine-4-carboxylic acid, (Compound 110)

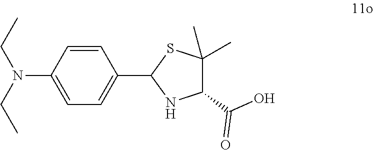 [SMILES: CCN(CC)c1ccc(C2N[C@@H](C(=O)O)C(C)(C)S2)cc1]

To a solution of 4-diethylaminobenzaldehyde, 10 (Alfa Aesar, 0.177 g, 1.0 mmol) in ethanol (5 mL) was added penicillamine (Cayman Chemical, 0.149 g, 1.0 mmol). The reaction mixture was stirred at 40 ºC for 16 hours under N₂ atmosphere. After stirring overnight, a white precipitate formed in the solution. Next the reaction mixture was diluted with ethanol (10 mL) and the solution was filtered over a fritted funnel. The filtered solid was washed with excess ethanol and then dried under reduce pressure to obtain the title compound as a white solid (211 mg, 0.68 mmol, 68% yield); R_{f} 0.06 with 1:1 v/v hexanes-ethyl acetate (UV 254 nM); ¹H-NMR (400 MHZ; DMSO-d₆) shows 70:30 mixture of enantiomers δ 7.22 (d, 2H, J=8.7 Hz), 7.13 (d, 1H, J=8.7 Hz), 6.5-6.6 (m, 3H), 5.74 (s, 1H), 5.47 (s, 1H), 3.2-3.4 (m, 8H), 1.59 (s, 3H, 1.52 (s, 1H), 1.29 (s, 3H), 1.26 (s, 1H), 1.0-1.1(m, 8H); MS (ES⁺) m/z 351.2 (M+1); HPLC UV purity, Rt=19.33 min, 99.66%; melting point=158.3-158.5° C.

### Preparation of Hydrazide Prodrugs

Hydrazide prodrugs useful for treating FGF-modulated diseases or injuries are synthesized from commercially available aldehydes 1a-z and commercially available hydrazide reagents 12a-z using the method shown in Scheme 9. The list of aldehydes 1a-z are provided in Table 1: The list of hydrazide reagents 12a-z and corresponding products 13a-z are provided in Table 7.

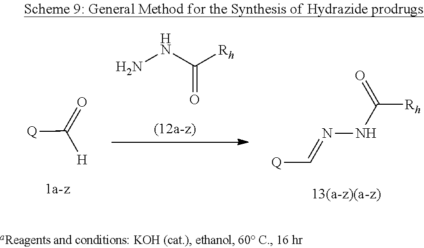

To a solution of aldehyde 1a-z (1 molar equivalents) in ethanol is added hydrazide reagents 12a-z (1 molar equivalents). One pellet of potassium hydroxide is added, and the reaction mixture is heated to 60° C. ovemight while under N₂ atmosphere. After reaction shows completion by disappearance of the staring material on TLC, the crude reaction mixture is diluted with ethanol and the precipitated solid is filtered over a fritted funnel which affords the hydrazide prodrugs 130(a-z) (Table 7). If needed, the crude product is purified by flash silica column chromatography on a CombiFlash NextGen 300+ purification system.

TABLE 7
Hydrazide reagents and hydrazide prodrugs

| # | Hydrazide Reagent Structure | # | Product of Hydrazide-aldehyde 1o Condensation |
|---|---|---|---|
| 12a | 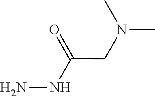 | 13oa | 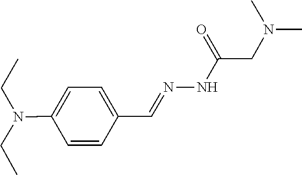 [SMILES: CCN(CC)c1ccc(/C=N/NC(=O)CN(C)C)cc1] |
| 12b | 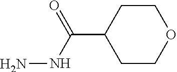 | 13ob | 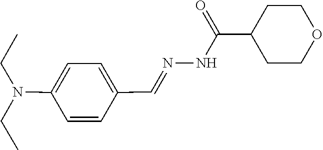 [SMILES: CCN(CC)c1ccc(/C=N/NC(=O)C2CCOCC2)cc1] |
| 12c | 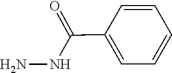 | 13oc | 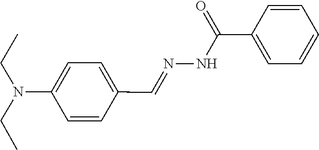 [SMILES: CCN(CC)c1ccc(/C=N/NC(=O)c2ccccc2)cc1] |
| 12d | 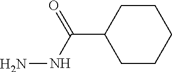 | 13od | 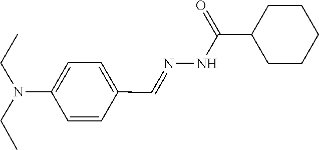 [SMILES: CCN(CC)c1ccc(/C=N/NC(=O)C2CCCCC2)cc1] |
| 12e | 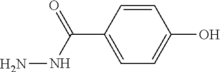 [SMILES: NNC(=O)c1ccc(O)cc1] | 13oe | 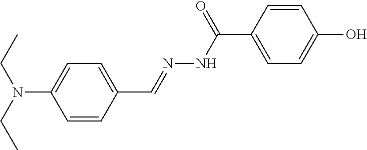 [SMILES: CCN(CC)c1ccc(/C=N/NC(=O)c2ccc(O)cc2)cc1] |
| 12f | 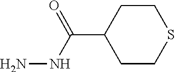 | 13of | 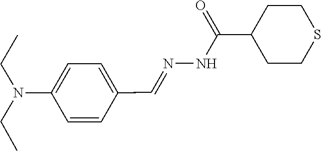 [SMILES: CCN(CC)c1ccc(/C=N/NC(=O)C2CCSCC2)cc1] |
| 12g | 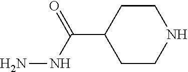 | 13og | 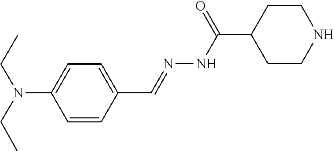 [SMILES: CCN(CC)c1ccc(/C=N/NC(=O)C2CCNCC2)cc1] |
| 12h | 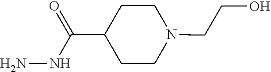 [SMILES: NNC(=O)C1CCN(CCO)CC1] | 13oh | 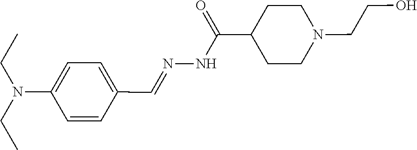 [SMILES: CCN(CC)c1ccc(/C=N/NC(=O)C2CCN(CCO)CC2)cc1] |
| 12i | 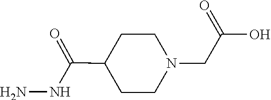 [SMILES: NNC(=O)C1CCN(CC(=O)O)CC1] | 13oi | 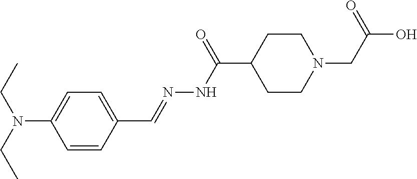 [SMILES: CCN(CC)c1ccc(/C=N/NC(=O)C2CCN(CC(=O)O)CC2)cc1] |
| 12j | 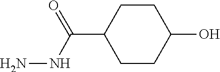 [SMILES: NNC(=O)C1CCC(O)CC1] | 13oj | 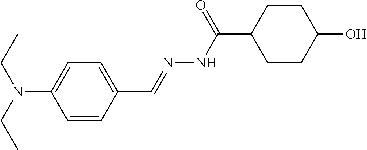 [SMILES: CCN(CC)c1ccc(/C=N/NC(=O)C2CCC(O)CC2)cc1] |
| 12k | 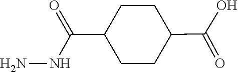 [SMILES: NNC(=O)C1CCC(C(=O)O)CC1] | 13ok | 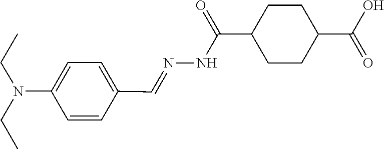 [SMILES: CCN(CC)c1ccc(/C=N/NC(=O)C2CCC(C(=O)O)CC2)cc1] |
| 12l | 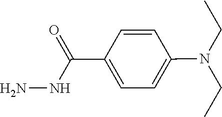 | 13ol | 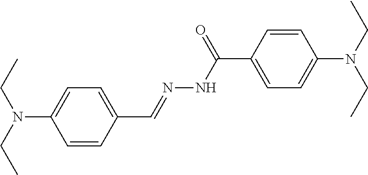 [SMILES: CCN(CC)c1ccc(/C=N/NC(=O)c2ccc(N(CC)CC)cc2)cc1] |
| 12m | 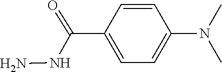 | 13om | 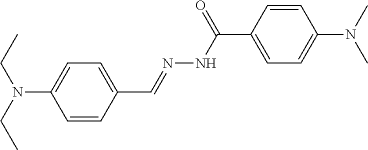 [SMILES: CCN(CC)c1ccc(/C=N/NC(=O)c2ccc(N(C)C)cc2)cc1] |
| 12n | 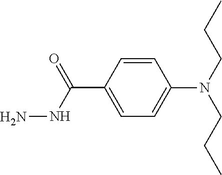 [SMILES: CCCN(CCC)c1ccc(C(=O)NN)cc1] | 13on | 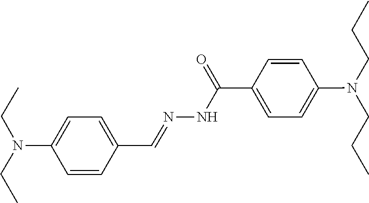 [SMILES: CCCN(CCC)c1ccc(C(=O)N/N=C/c2ccc(N(CC)CC)cc2)cc1] |
| 12o | 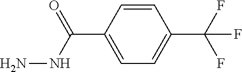 [SMILES: NNC(=O)c1ccc(C(F)(F)F)cc1] | 13oo | 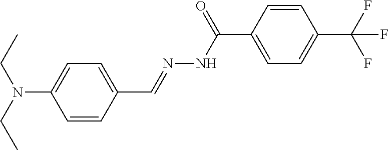 [SMILES: CCN(CC)c1ccc(/C=N/NC(=O)c2ccc(C(F)(F)F)cc2)cc1] |
| 12p | 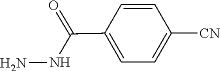 [SMILES: N#Cc1ccc(C(=O)NN)cc1] | 13op | 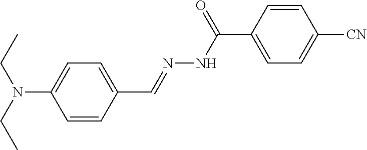 [SMILES: CCN(CC)c1ccc(/C=N/NC(=O)c2ccc(C#N)cc2)cc1] |
| 12q | 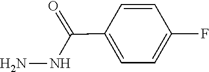 [SMILES: NNC(=O)c1ccc(F)cc1] | 13oq | 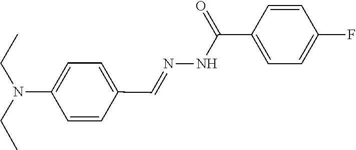 [SMILES: CCN(CC)c1ccc(/C=N/NC(=O)c2ccc(F)cc2)cc1] |
| 12r | 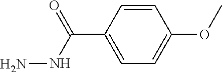 | 13or | 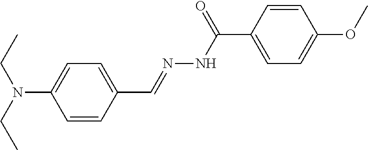 [SMILES: CCN(CC)c1ccc(/C=N/NC(=O)c2ccc(OC)cc2)cc1] |
| 12s | 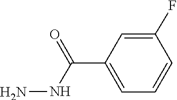 [SMILES: NNC(=O)c1cccc(F)c1] | 13os | 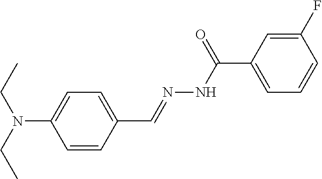 [SMILES: CCN(CC)c1ccc(/C=N/NC(=O)c2cccc(F)c2)cc1] |
| 12t | 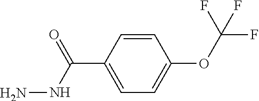 [SMILES: NNC(=O)c1ccc(OC(F)(F)F)cc1] | 13ot | 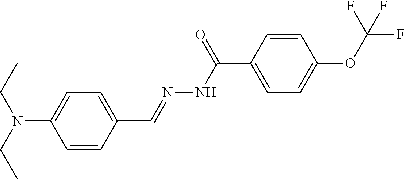 [SMILES: CCN(CC)c1ccc(/C=N/NC(=O)c2ccc(OC(F)(F)F)cc2)cc1] |
| 12u | 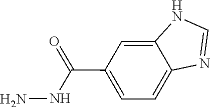 [SMILES: NNC(=O)c1ccc2nc[nH]c2c1] | 13ou | 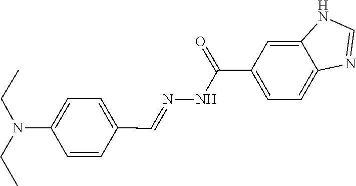 [SMILES: CCN(CC)c1ccc(/C=N/NC(=O)c2ccc3nc[nH]c3c2)cc1] |
| 12v | 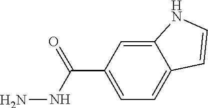 [SMILES: NNC(=O)c1ccc2cc[nH]c2c1] | 13ov | 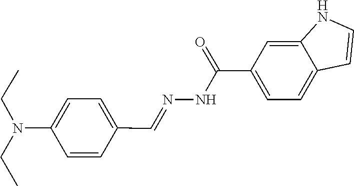 [SMILES: CCN(CC)c1ccc(/C=N/NC(=O)c2ccc3cc[nH]c3c2)cc1] |
| 12w | 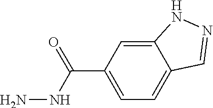 [SMILES: NNC(=O)c1ccc2cn[nH]c2c1] | 13ow | 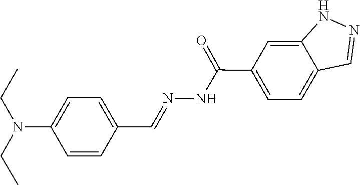 [SMILES: CCN(CC)c1ccc(/C=N/NC(=O)c2ccc3cn[nH]c3c2)cc1] |
| 12x | 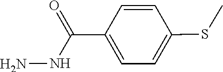 | 13ox | 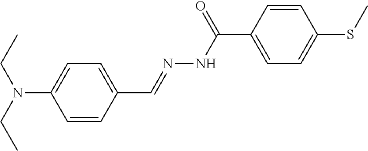 [SMILES: CCN(CC)c1ccc(/C=N/NC(=O)c2ccc(SC)cc2)cc1] |
| 12y | 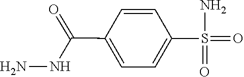 [SMILES: NNC(=O)c1ccc(S(N)(=O)=O)cc1] | 13oy | 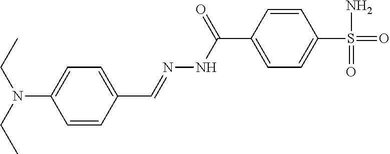 [SMILES: CCN(CC)c1ccc(/C=N/NC(=O)c2ccc(S(N)(=O)=O)cc2)cc1] |
| 12z | 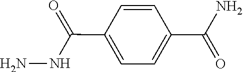 [SMILES: NNC(=O)c1ccc(C(N)=O)cc1] | 13oz | 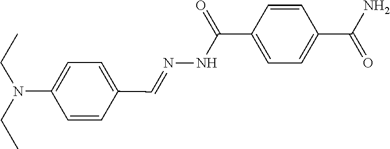 [SMILES: CCN(CC)c1ccc(/C=N/NC(=O)c2ccc(C(N)=O)cc2)cc1] |

### Preparation of (E)-N′-(4-(diethylamino)benzylidene)-2-(dimethylamino)acetohydrazide, (Compound 130a)

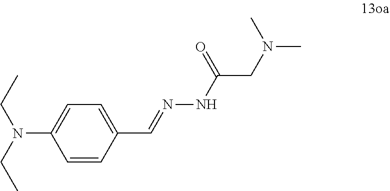 [SMILES: CCN(CC)c1ccc(/C=N/NC(=O)CN(C)C)cc1]

To a solution of 4-diethylaminobenzaldehyde, 10 (Alfa Aesar, 0.300 g, 1.68 mmol) in ethanol (30 mL) was added D-Glucosamine Hydrochloride, 12a (Cayman Chemical, 0.198 g, 1.68 mmol). One pellet of potassium hydroxide was added, and the reaction mixture was heated to 60° C. overnight while under N₂ atmosphere. After reaction shows completion by disappearance of the staring material on TLC, the crude reaction mixture was diluted with ethanol and the precipitated solid was filtered over a fritted funnel which affords the crude compound. The crude product was purified by flash silica column chromatography on a CombiFlash NextGen 300+ purification system. A 40 g RediSep Gold R_{f} column was pre-conditioned by eluting with 1% MeOH/DCM over 3 column volumes. Elution occurred with methanol (Solvent A) and dichloromethane using a gradient of 1-100% (Solvent A) over 15 column volumes. After collecting appropriate fractions from the column, the combined fractions were concentrated to obtain the title compound as a white solid (315 mg, 0.68 mmol, 68% yield); R_{f} 0.50 with 10:90 v/v methanol-dichloromethane (UV 254 nM); ¹H-NMR (400 MHZ; DMSO-d₆) E/Z mixture δ 10.84 (s, 1H), 8.12 (s, 1H), 7.3-7.4 (m, 2H), 6.6-6.7 (m, 2H), 6.5-6.6 (m, 3H), 3.2-3.4 (m, 4H), 2.2-2.3 (m, 6H), 1.0-1.1(m, 6H); MS (ES⁺) m/z 277.3 (M+1); HPLC UV purity, Rt =10.097 min, 98.46%; melting point =107-108° C.

### Preparation of (E)-N′-(4-(diethylamino)benzylidene)tetrahydro-2H-pyran-4-carbohydrazide, (Compound 13ob)

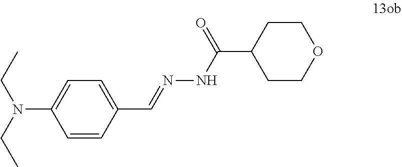 [SMILES: CCN(CC)c1ccc(/C=N/NC(=O)C2CCOCC2)cc1]

To a solution of 4-diethylaminobenzaldehyde, 10 (Alfa Aesar, 0.077 g, 0.44 mmol) in ethanol (3 mL) was added oxone-4-carbohydrazide, 12b (Combi-Blocks, 0.050 g, 0.44 mmol). One pellet of potassium hydroxide was added, and the reaction mixture was heated to 60° C. overnight while under N₂ atmosphere. After reaction shows completion by disappearance of the staring material on TLC, the crude reaction mixture was diluted with ethanol and the precipitated solid was filtered over a fritted funnel which affords the crude compound. The crude product was purified by flash silica column chromatography on a CombiFlash NextGen 300+ purification system. A 40 g RediSep Gold R_{f} column was pre-conditioned by eluting with 40% EA/Heptane over 3 column volumes. Elution occurred with ethyl acetate (Solvent A) and heptane using a gradient of 40-60% (Solvent A) over 15 column volumes. After collecting appropriate fractions from the column, the combined fractions were concentrated to obtain the title compound as a amber solid (86 mg, 0.28 mmol, 65% yield); R_{f} 0.50 with 10:90 v/v methanol-dichloromethane (UV 254 nM); ¹H-NMR (400 MHz; DMSO-d₆) E/Z mixture δ 10.98, 10.8-10.9 (s, 1H), 7.8-8.0(s, 1H), 7.39 (br t, 2H, J=8.3 Hz), 6.9-7.2 (m, 1H), 6.64 (d, 2H, J=8.3 Hz), 3.85 (d, 2H, J=11.0 Hz), 3.3-3.4 (m, 4H), 1.5-1.7 (m, 4H), 1.0-1.1(dt, 6H, J=2.3, 6.9 Hz); MS (APCI⁺) m/z 304.3 (M+1); HPLC UV purity, Rt =16.90 min, 96.41%

### Preparation of (E)-N′-(4-(diethylamino)benzylidene)-1-(2-hydroxyethyl)piperidine-4-carbohydrazide, (Compound 13oh)

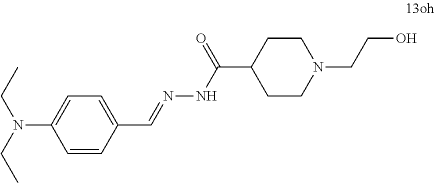 [SMILES: CCN(CC)c1ccc(/C=N/NC(=O)C2CCN(CCO)CC2)cc1]

To a solution of 4-diethylaminobenzaldehyde, 10 (Alfa Aesar, 0.491 g, 2.8 mmol) in ethanol (3 mL) was added 1-(2-hydroxyethyl)piperidine-4-carbohydrazide, 12h (Aurora, 0.173 g, 0.92 mmol). Molecular sieves 5 Å was added, and the reaction mixture was stirred overnight while under N₂ atmosphere. After reaction shows completion by disappearance of the staring material on TLC, the crude reaction mixture was diluted with ethanol and the molecular sieves was filtered over a fritted funnel which affords the crude compound. The crude product was purified by flash silica column chromatography on a CombiFlash NextGen 300+ purification system. A 24 g RediSep Gold R_{f} column was pre-conditioned by eluting with 1% TEA/Methanol over 3 column volumes. Elution occurred with methanol/TEA (1%)

(Solvent A) and Dichlormethane (Solvent B) (1%) using a gradient of 1-100% (Solvent A) over 20 column volumes. After collecting appropriate fractions from the column, the combined fractions were concentrated to obtain the title compound as a yellow solid (205 mg, 0.28 mmol, 64% yield); R_{f} 0.45 with 10:90 v/v methanol-dichloromethane (UV 254 nM); ¹H-NMR (400 MHZ; DMSO-d₆) E/Z mixture δ 10.97 (s, 0.5H), 10.84 (s, 0.5H), 7.99(s, 0.5H), 7.81 (s, 0.5 H), 7.42 (t, 2H, J=9.4 Hz), 6.67 (d, 2H, J=8.7 Hz), 4.38 (br s, 1H), 3.3-3.5 (m, 6H), 2.9-3.1 (m, 3.0 H), 1.5-1.7 (m, 4H), 1.0-1.1(m, 6H); MS (APCI⁺) m/z 347.3 (M+1); HPLC UV purity, Rt=20.15 min, 95.2%; melting point 88-90° C. (decomposition).

### Preparation of Hydrazone Prodrugs

Hydrazone prodrugs useful for treating FGF-modulated diseases or injuries are synthesized from commercially available aldehydes 1a-z and commercially available hydrazine reagents 14a-z using the method shown in Scheme 10. The list of aldehydes 1a-z are provided in Table 1. The list of hydrazine reagents 14a-z and corresponding products 15a-z are provided in Table 8.

### Scheme 10: General Method for the Synthesis of Hydrazone prodrugs

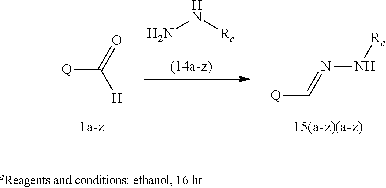

5 To a solution of aldehyde 1a-z (1 molar equivalents) in ethanol is added hydrazine reagents 14a-z (1 molar equivalents). The reaction mixture is stirred overnight while under N₂ atmosphere. After reaction shows completion by disappearance of the staring material on TLC, the crude reaction mixture is diluted with ethanol and the precipitated solid is filtered over a fritted funnel which affords the hydrazone prodrugs 15(a-z)(a-z) (Table 8). If needed, the crude product is purified by flash silica column chromatography on a CombiFlash NextGen 300+ purification system.

TABLE 8
Hydrazine reagents and hydrazone prodrug products

| # | Structure of Hydrazine Reagent | # | Product of Hydrazine-Aldehyde 1o Condensation |
|---|---|---|---|
| 14a | 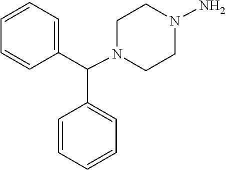 [SMILES: NN1CCN(C(c2ccccc2)c2ccccc2)CC1] | 15oa | 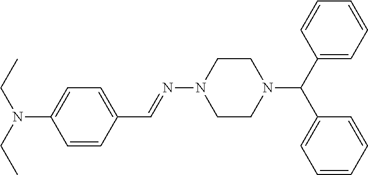 [SMILES: CCN(CC)c1ccc(/C=N/N2CCN(C(c3ccccc3)c3ccccc3)CC2)cc1] |
| 14b | 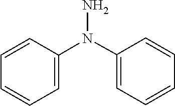 [SMILES: NN(c1ccccc1)c1ccccc1] | 15ob | 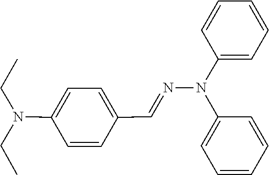 [SMILES: CCN(CC)c1ccc(/C=N/N(c2ccccc2)c2ccccc2)cc1] |
| 14c | 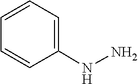 | 15oc | 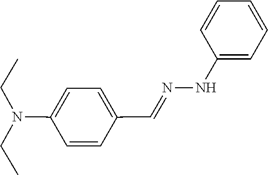 [SMILES: CCN(CC)c1ccc(/C=N/Nc2ccccc2)cc1] |
| 14d | 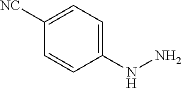 | 15od | 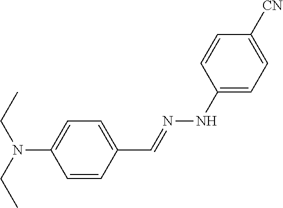 [SMILES: CCN(CC)c1ccc(/C=N/Nc2ccc(C#N)cc2)cc1] |
| 14e | 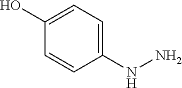 | 15oe | 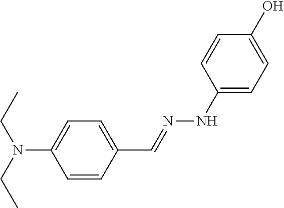 [SMILES: CCN(CC)c1ccc(/C=N/Nc2ccc(O)cc2)cc1] |
| 14f | 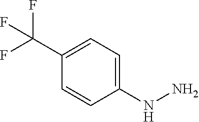 [SMILES: NNc1ccc(C(F)(F)F)cc1] | 15of | 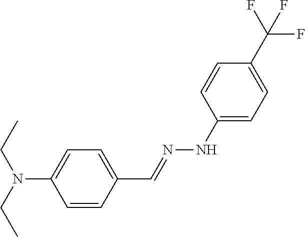 [SMILES: CCN(CC)c1ccc(/C=N/Nc2ccc(C(F)(F)F)cc2)cc1] |
| 14g | 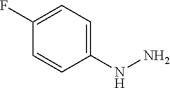 | 15og | 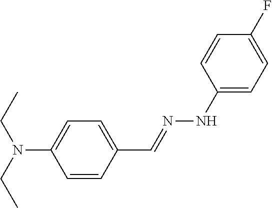 [SMILES: CCN(CC)c1ccc(/C=N/Nc2ccc(F)cc2)cc1] |
| 14h | 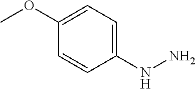 | 15oh | 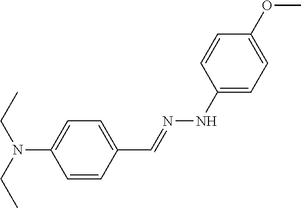 [SMILES: CCN(CC)c1ccc(/C=N/Nc2ccc(OC)cc2)cc1] |
| 14i | 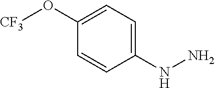 [SMILES: NNc1ccc(OC(F)(F)F)cc1] | 15oi | 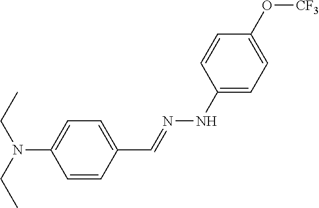 [SMILES: CCN(CC)c1ccc(/C=N/Nc2ccc(OC(F)(F)F)cc2)cc1] |
| 14j | 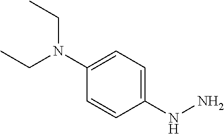 | 15oj | 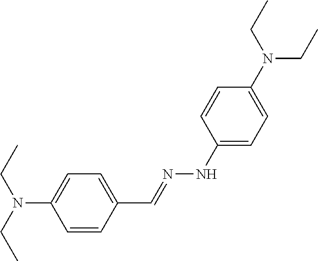 [SMILES: CCN(CC)c1ccc(/C=N/Nc2ccc(N(CC)CC)cc2)cc1] |
| 14k | 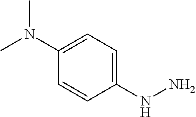 | 15ok | 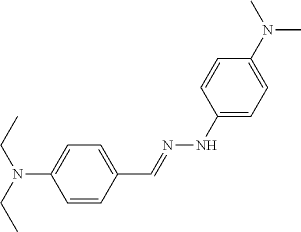 [SMILES: CCN(CC)c1ccc(/C=N/Nc2ccc(N(C)C)cc2)cc1] |
| 14l | 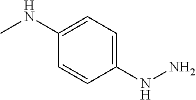 | 15ol | 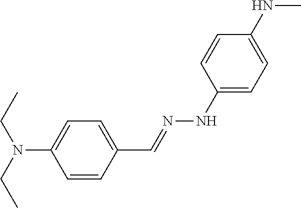 [SMILES: CCN(CC)c1ccc(/C=N/Nc2ccc(NC)cc2)cc1] |
| 14m | 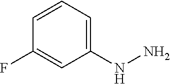 | 15om | 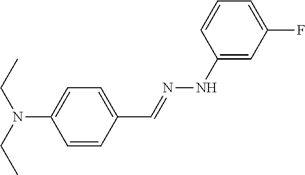 [SMILES: CCN(CC)c1ccc(/C=N/Nc2cccc(F)c2)cc1] |
| 14n | 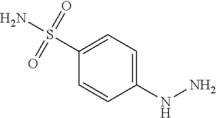 [SMILES: NNc1ccc(S(N)(=O)=O)cc1] | 15on | 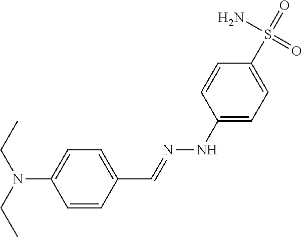 [SMILES: CCN(CC)c1ccc(/C=N/Nc2ccc(S(N)(=O)=O)cc2)cc1] |
| 14o | 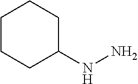 | 15oo | 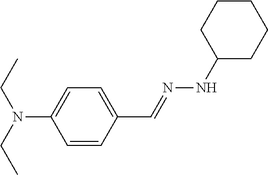 [SMILES: CCN(CC)c1ccc(/C=N/NC2CCCCC2)cc1] |
| 14p | 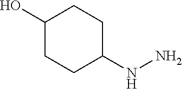 | 15op | 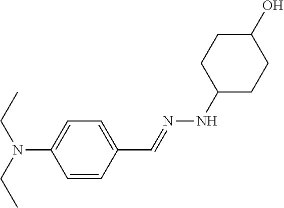 [SMILES: CCN(CC)c1ccc(/C=N/NC2CCC(O)CC2)cc1] |
| 14q | 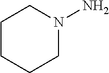 | 15oq | 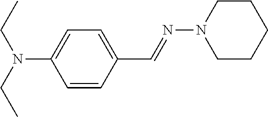 [SMILES: CCN(CC)c1ccc(/C=N/N2CCCCC2)cc1] |
| 14r | 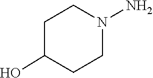 | 15or | 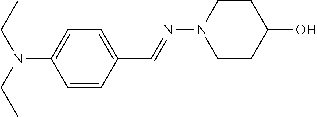 [SMILES: CCN(CC)c1ccc(/C=N/N2CCC(O)CC2)cc1] |
| 14s | 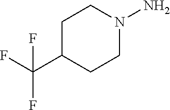 [SMILES: NN1CCC(C(F)(F)F)CC1] | 15os | 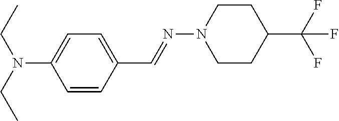 [SMILES: CCN(CC)c1ccc(/C=N/N2CCC(C(F)(F)F)CC2)cc1] |
| 14t | 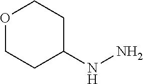 | 15ot | 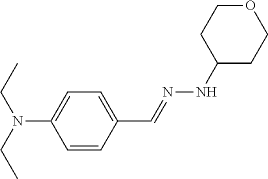 [SMILES: CCN(CC)c1ccc(/C=N/NC2CCOCC2)cc1] |
| 14u | 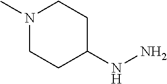 | 15ou | 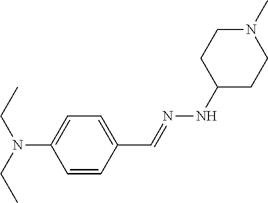 [SMILES: CCN(CC)c1ccc(/C=N/NC2CCN(C)CC2)cc1] |
| 14v | 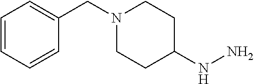 [SMILES: NNC1CCN(Cc2ccccc2)CC1] | 15ov | 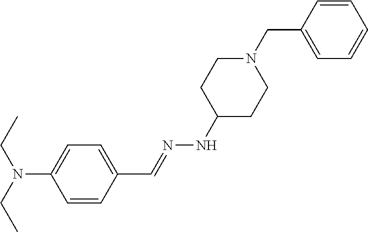 [SMILES: CCN(CC)c1ccc(/C=N/NC2CCN(Cc3ccccc3)CC2)cc1] |
| 14w | 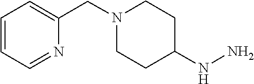 [SMILES: NNC1CCN(Cc2ccccn2)CC1] | 15ow | 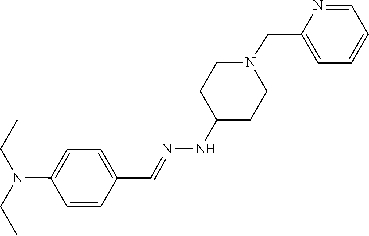 [SMILES: CCN(CC)c1ccc(/C=N/NC2CCN(Cc3ccccn3)CC2)cc1] |
| 14x | 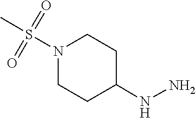 | 15ox | 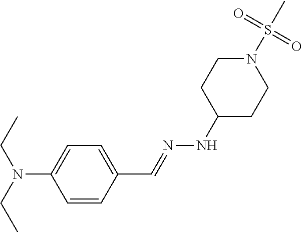 [SMILES: CCN(CC)c1ccc(/C=N/NC2CCN(S(C)(=O)=O)CC2)cc1] |
| 14y | 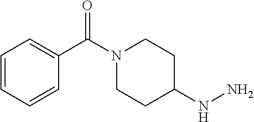 [SMILES: NNC1CCN(C(=O)c2ccccc2)CC1] | 15oy | 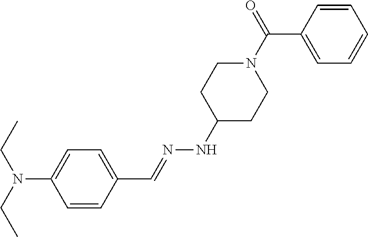 [SMILES: CCN(CC)c1ccc(/C=N/NC2CCN(C(=O)c3ccccc3)CC2)cc1] |
| 14z | 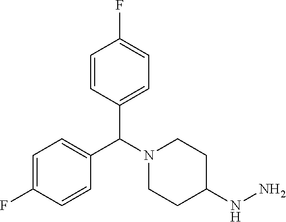 [SMILES: NNC1CCN(C(c2ccc(F)cc2)c2ccc(F)cc2)CC1] | 15oz | 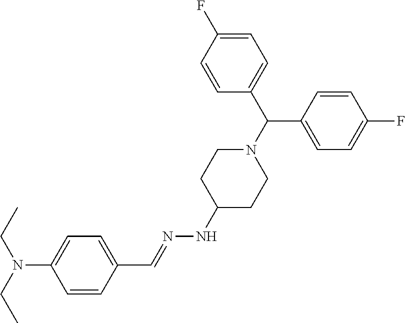 [SMILES: CCN(CC)c1ccc(/C=N/NC2CCN(C(c3ccc(F)cc3)c3ccc(F)cc3)CC2)cc1] |

### Preparation of (E)-4-(((4-benzhydrylpiperazin-1-yl)imino)methyl)-N,N-diethylaniline, (Compound 15oa)

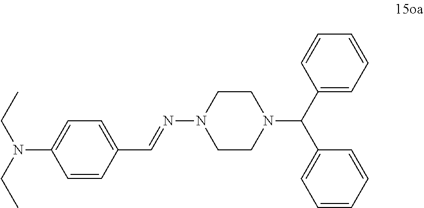 [SMILES: CCN(CC)c1ccc(/C=N/N2CCN(C(c3ccccc3)c3ccccc3)CC2)cc1]

To a solution of 4-diethylaminobenzaldehyde, 10 (Alfa Aesar, 0.840 g, 3.1 mmol) in ethanol (3 mL) was added 4-(diphenylmethyl)piperazin-1-amine, 14a (Enamine, 0.567 g, 3.1 mmol). The reaction mixture was stirred overnight at room temperature while under N₂ atmosphere. After reaction shows completion by disappearance of the staring material on TLC, the crude reaction mixture was diluted with ethanol (2 mL) and water (5 mL) and the precipitated solid was filtered over a fritted funnel which affords the title compound as white solid (1.15 g, 2.69 mmol, 86% yield); R_{f} 0.42 with 30:70 v/v ethyl acetate-heptane (UV 254 nM); ¹H-NMR (400 MHZ; DMSO-d₆) δ 7.50 (s, 1H), 7.4-7.5 (m, 4H) 7.3-7.4 (m, 6H), 7.1-7.2 (m, 2H), 6.62 (d, 2H,

J=9.0 Hz), 4.34 (s, 1H), 3.3-3.4 (m, 4H), 3.03 (br s, 4H), 2.4-2.5 (m, 4H), 1.05 (t, 6H, J=7.0 Hz); MS (ESI+) m/z 427.25 (M+1); HPLC UV purity, Rt =12.173 min, 98.35%; melting point 124.5-126.4° C.

Preparation of (E)-4-(2-(4-(diethylamino)benzylidene)hydrazineyl)benzonitrile, (Compound 15od)

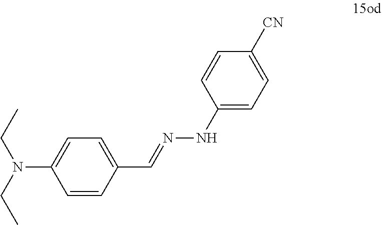 [SMILES: CCN(CC)c1ccc(/C=N/Nc2ccc(C#N)cc2)cc1]

To a solution of 4-diethylaminobenzaldehyde, 10 (Alfa Aesar, 0.134 g, 0.75 mmol) in ethanol (3 mL) was added 4-hydrazinylbenzonitrile, 14d (Bepharm Scientific, 0.100 g, 0.75 mmol). The reaction mixture was stirred overnight at room temperature while under N₂ atmosphere. After reaction shows completion by disappearance of the staring material on TLC, the crude reaction mixture was diluted with ethanol (2 mL) and water (5 mL) and the precipitated solid was filtered over a fritted funnel which affords the title compound as yellow solid (189 mg, 0.65 mmol, 86% yield); R_{f} 0.42 with 30:70 v/v ethyl acetate-heptane (UV 254 nM); ¹H-NMR (400 MHZ; DMSO-d₆) δ 10.98, 10.60 (s, 1H), 7.83 (s, 1H), 7.55 (d, 2H, J=8.3 Hz) 7.46 (d, 2H, J=8.7 Hz), 7.06 (d, 2H, J=8.7 Hz), 6.67 (d, 2H, J=9.2 Hz), 3.3-3.4 (m, 4H), 1.0-1.1(m, 6H); MS (ESI+) m/z 293.1 (M+1); HPLC UV purity, Rt=11.96 min, 99.71%; melting point 155-157° C.

### Example 2. Thermal Shift Assay (TSA)

TSA was utilized to biophysically characterize the recombinant human FGFR1/FGF2 complex in the presence or absence of selected compounds of formula (I), Compounds 1, 2a-2f, 20, 5, 60, and 8-13. Compound 1 was prepared according to the procedures described in Example 1, and the other compounds were obtained from commercial sources. The assay functions by protein denaturation over a temperature gradient. During protein unfolding, exposed hydrophobic regions bind a dye and fluoresce due to solvent relaxation effects. Changes in the melting temperature of the protein complex in the presence of each compound were monitored and compounds were screened/ranked using this method.

### FGFR₁ Protein Expression and Purification

One Shot BL21 (DE3) Star Escherichia coli competent cells (Thermo Fisher) were transformed with the relevant FGFR₁ plasmid and inoculated onto Ampicillin Luria Broth/Agar plates. Two hundred milliliter portions of Terrific Broth starter cultures were used to inoculate 9 L cultures with ampicillin at a concentration of 100 μg/mL. Cultures were grown to an O.D.600 near 1.0 at 37° C. and induced with isopropyl β-D-1-thiogalactopyranoside (IPTG) for 5 hours at 37° C. The cells were then harvested by centrifugation using a F9-6×1000 LEX rotor at 6000 rpm for 10 min at 4° C. in a Sorvall Lynx 6000 centrifuge (Thermo Scientific). Bacterial pellets were stored at −80° C. until use.

Cell pellets were thawed and resuspend in 100 mL of FGFR₁ Lysis Buffer per 9 g of pellet (20 mM Tris-HCl PH 8.0, 500 mM NaCl, 1 mM dithiothreitol) by stirring at 4° C. for 1 hour. Cells were lysed in 3 cycles on/off for 3 minutes each at 4° C. via sonication followed by centrifugation for 30 minutes at 16,000 RPM in rotor F20 at 4° C., after which the supernatant was discarded. This process was then repeated twice. The pellets were resuspended in 150 mL FGFR₁ solubilization buffer (8 M urea, 20 mM Tris-HCL pH 8.0, 150 mM NaCl, 1 mM dithiothreitol) by stirring for 1 hour at 4° C., and the solution was subjected to centrifugation for 30 minutes at 16,000 RPM in rotor F20 at 4° C. The pellets were discarded, and the supernatant was filtered through a 0.45 μM polyethersylfone (PES) filter. After filtration, the supernatant was added dropwise to 1 L FGFR₁ refolding buffer (20 mM Tris-HCl PH 8.0, 150 mM NaCl, 0.5 M L-arginine, 25 mM MgCl₂) using a glass column. Protein was concentrated by tangential flow from 1 L to 100 mL and dialyzed against 1 L of FGFR₁ Dialysis Buffer (20 mM Tris-HCl pH 8.0, 150 mM NaCl, 25 mM MgCl₂) for 2 hours at 4° C., and the dialysis step was repeated with fresh buffer for an additional 2 hours at 4° C. The material thus obtained was then centrifuged at 4000 RPM in Eppendorf tabletop centrifuge for 5 minutes and loaded onto 2×5mL heparin columns. The columns were washed extensively (20 CV) using FGFR₁ Heparin Buffer A (20 mM Tris-HCl PH 8.0, 150 mM NaCl, 25 mM MgCl₂) and then eluted using FGFR₁ Heparin Buffer B (20 mM Tris-HCl PH 8.0, 1.5 M NaCl, 25 mM MgCl₂). A large peak was recovered that was >95% pure by SDS-PAGE analysis gel (Expected Mw: 25 KDa). The protein was collected and diluted in 20 mM Tris-HCl PH 8.0, 25 mM MgCl₂ buffer in order to reach a NaCl concentration of 150 mM. The FGFR₁ thus obtained was concentrated and stored at −80° C.

### FGF2 Protein Expression and Purification

One Shot BL21 (DE3) Star Escherichia coli competent cells (Thermo Fisher) were transformed with a relevant FGF2 plasmid and inoculated onto Ampicillin Luria Broth/Agar plates. Two hundred milliliter portions of Terrific Broth starter cultures were used to inoculate 9 L cultures with ampicillin at a concentration of 100 μg/mL. Cultures were grown to an O.D.600 near 1.0 at 37° C., and induced with IPTG overnight at 18° C. The cells were harvested at 7000 RPM in rotor 6000 for 5 min at 4° C. and stored at −80° C. Bacterial pellets were resuspended in 25 mM Hepes-NaOH, pH 7.5, 250 mM NaCl, and the cells were lysed in 3 cycles on/off for 3 minutes each at 4° C. via sonication. After centrifugation for 30 minutes at 16,000 RPM at 4° C. , the isolated pellets were discarded, and the supernatant was filtered supernatant through a 0.45 M PES filter using 100 mL superloop. The lysate was purified over a 5 mL S column by washing the column with Lysis buffer for 5 CV then eluting using gradient from 250 mM to 1 M NaCl over 20 CV. The fractions containing FGF2 were identified via SDS-PAGE gel (Expected Mw: 15.2 KDa). The protein was collected and diluted in 20 mM Tris-HCl PH 8.0, 25 mM MgCl₂ buffer in order to reach a NaCl concentration of 150 mM. The purified FGF2 was concentrated and stored at −80° C.

### FGFR1/FGF-2 Complex Formation and TSA protocol

Thawed aliquots of purified FGF2 (1.0 mg/mL) and FGFR₁ (1.6 mg/mL) proteins were mixed in a 1:1 molar ratio (64 uM: 64 μM) on ice for 30 min at 4° C. and plated prior to the thermal shift assay (TSA).

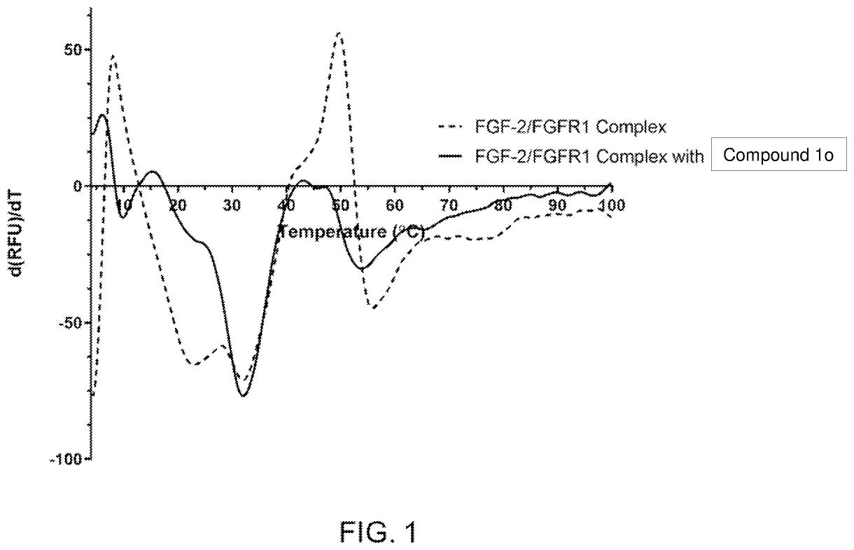

Complex formation was verified by loading the complexed material on a size exclusion column (superdex 10 300GL S200) and observing the monodisperse peak corresponding to the FGF2/FGFR₁ complex (˜ 40 kDa). Compounds described herein were screened in dose response format (0-100 μM) with the FGF2/FGFR₁ complex in triplicates. FGF2/ FGFR1/compound complexes were mixed in a 1000:1 ratio with Sypro Orange dye (Sigma-Aldrich). The samples were processed using a Bio-Rad CFX C₉₆ Touch quantitative polymerase chain reaction and run using the FRET assay settings with a heating ramp of 0.3° C./s cycling from 4 to 100° C. Data analysis was performed using the Bio-Rad CFX Manager Software (version 3.1, Bio-Rad) and changes in the melting temperature (Tm) of the complex in the presence of each compound were monitored. The results are shown in Table 9 below and also in FIG. 1 (for Compound 10).

FIG. 1 shows a thermal stability assay (TSA) of the purified FGF-2/FGFR₁ complex with and without Compound 10. The curve of the complex alone (dotted line) shows two positive peaks, one corresponding to FGF-2 (left) and one to FGFR₁ (right). In the presence of 25 μM Compound 10 (solid line), the TSA shows a shift of the melting curve, in effect moving the peaks closer together. This indicates binding of Compound 10 and increased stability of the complex.

TABLE 9
TSA Results for Selected Compounds of Formula (I)

| | | ΔTm (° C.) | | ΔTm (° C.) | | Commercial |
|---|---|---|---|---|---|---|
| Cmpd | Structure | −FGFR1 | | −FGF2 | | Source |
| 1a | 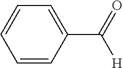 | +0.5 | (100 μM) | 0 | (100 μM) | Sigma Aldrich |
| 1b | 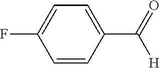 | 0 | (100 μM) | +1.0 | (100 μM) | Oakwood |
| 1c | 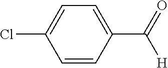 | −2.2 | (10 μM) | +2.0 | (10 μM) | Combi-Blocks |
| 1d | 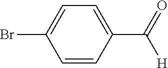 | −2.1 | (25 μM) | +1.6 | (25 μM) | ChemImpex |
| 1e | 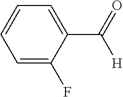 | 0 | (100 μM) | +0.3 | (100 μM) | AK Scientific |
| 1f | 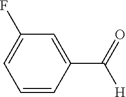 | +4.0 | (50 μM) | −0.4 | (50 μM) | Alfa Aesar |
| 1o | 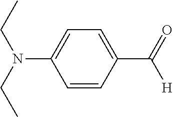 | +5.0 | (25 μM) | −2.0 | (25 μM) | TCI |
| 4on | 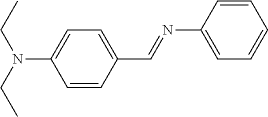 [SMILES: CCN(CC)c1ccc(/C=N/c2ccccc2)cc1] | +1.5 | (25 μM) | −0.5 | (25 μM) | Aldrich |
| 4ow | 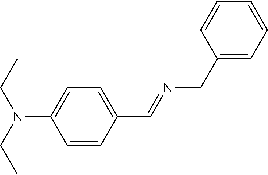 | +4.0 | (25 μM) | −2.0 | (25 μM) | — |
| 4oy | 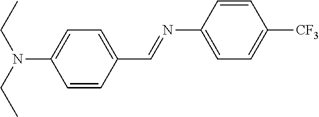 [SMILES: CCN(CC)c1ccc(/C=N/c2ccc(C(F)(F)F)cc2)cc1] | +7.8 | (50 μM) | −4.5 | (50 μM) | — |
| 5o | 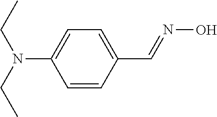 | +3.5 | (2 μM) | −1.0 | (2 μM) | Enamine |
| 8om | 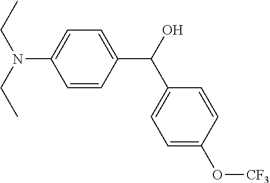 [SMILES: CCN(CC)c1ccc(C(O)c2ccc(OC(F)(F)F)cc2)cc1] | +0.3 | (10 μM) | 0 | (10 μM) | — |
| 9af | 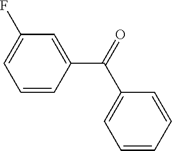 [SMILES: O=C(c1ccccc1)c1cccc(F)c1] | +9.8 | (10 μM) | −6.0 | (10 μM) | Oakwood |
| 9oa | 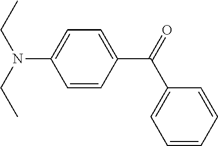 [SMILES: CCN(CC)c1ccc(C(=O)c2ccccc2)cc1] | +6.0 | (10 μM) | −0.3 | (10 μM) | Toronto Research |
| 9ob | 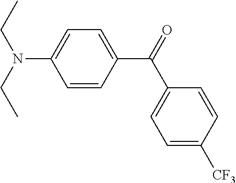 [SMILES: CCN(CC)c1ccc(C(=O)c2ccc(C(F)(F)F)cc2)cc1] | +1.2 | (50 μM) | +1.5 | (50 μM) | — |
| 9oc | 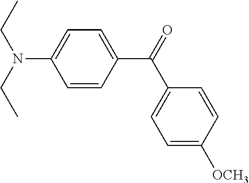 [SMILES: CCN(CC)c1ccc(C(=O)c2ccc(OC)cc2)cc1] | +9.5 | (2 μM) | −5.6 | (2 μM) | — |
| 9om | 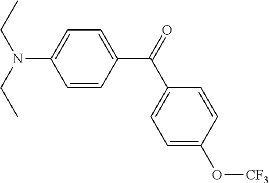 [SMILES: CCN(CC)c1ccc(C(=O)c2ccc(OC(F)(F)F)cc2)cc1] | +1.2 | (100 μM) | +1.8 | (100 μM) | — |
| 10o | 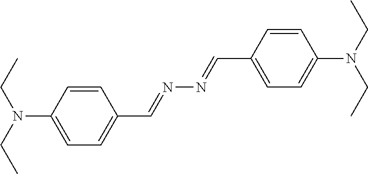 [SMILES: CCN(CC)c1ccc(/C=N/N=C/c2ccc(N(CC)CC)cc2)cc1] | +11.0 | (2 μM) | −5.2 | (2 μM) | — |
| 11o | 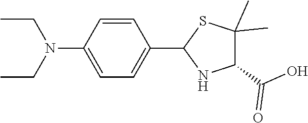 [SMILES: CCN(CC)c1ccc(C2N[C@@H](C(=O)O)C(C)(C)S2)cc1] | +2.0 | (50 μM) | −4.2 | (50 μM) | — |
| 13oa | 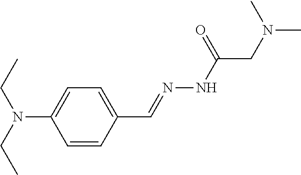 [SMILES: CCN(CC)c1ccc(/C=N/NC(=O)CN(C)C)cc1] | 0 | (50 μM) | −2.1 | (50 μM) | — |
| 13ob | 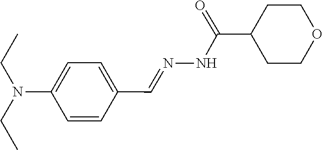 [SMILES: CCN(CC)c1ccc(/C=N/NC(=O)C2CCOCC2)cc1] | +1.5 | (25 μM) | −2.7 | (25 μM) | — |
| 13oe | 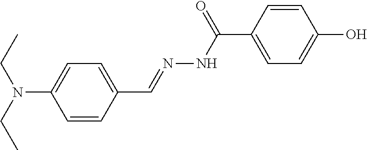 [SMILES: CCN(CC)c1ccc(/C=N/NC(=O)c2ccc(O)cc2)cc1] | −1.5 | (10 μM) | −0.5 | (10 μM) | Cayman |
| 13oh | 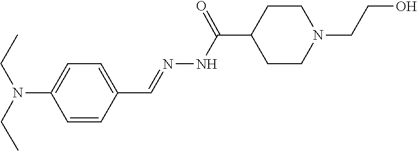 [SMILES: CCN(CC)c1ccc(/C=N/NC(=O)C2CCN(CCO)CC2)cc1] | +0.6 | (10 μM) | 0 | (10 μM) | — |
| 15oa | 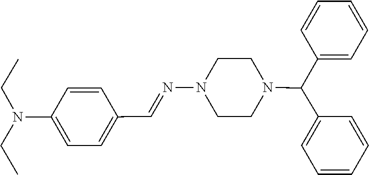 [SMILES: CCN(CC)c1ccc(/C=N/N2CCN(C(c3ccccc3)c3ccccc3)CC2)cc1] | +2.0 | (100 μM) | −1.5 | (100 μM) | — |
| 15ob | 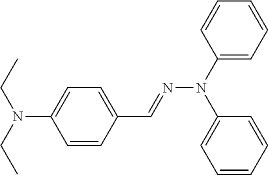 [SMILES: CCN(CC)c1ccc(/C=N/N(c2ccccc2)c2ccccc2)cc1] | 0 | (25 μM) | −1.0 | (25 μM) | TCI |
| 15oc | 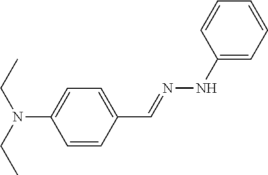 [SMILES: CCN(CC)c1ccc(/C=N/Nc2ccccc2)cc1] | +4.3 | (2 μM) | −2.4 | (2 μM) | Aldrich |
| 15od | 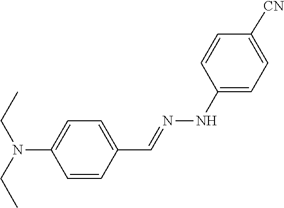 [SMILES: CCN(CC)c1ccc(/C=N/Nc2ccc(C#N)cc2)cc1] | 0 | (10 μM) | −1.0 | (10 μM) | — |
| 16 | 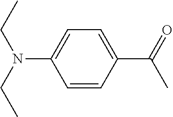 | +0.2 | (25 μM) | 0 | (25 μM) | Alfa Aesar |
| 17 | 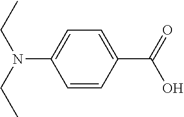 | +2.5 | (100 μM) | 0 | (100 μM) | Oakwood |

### Example 3. Effects of Compound 10 on the Phosphorylation of FGFR1

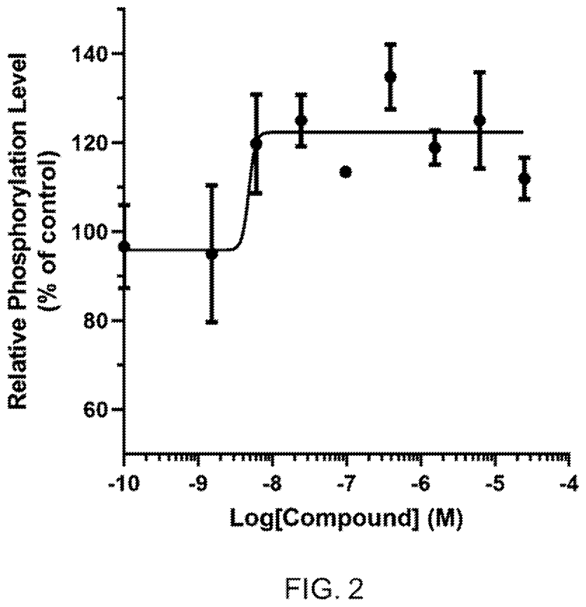

Cells expressing FGFR₁ were exposed to increasing concentrations of Compound 10 in the presence of a submaximal concentration of FGF-2. Cells were then lysed, and the relative phosphorylation of FGFR₁ was assessed using antibodies to non-phosphorylated and phosphorylated FGFR1. The results are shown in FIG. 2, which is a graph showing the phosphorylation of FGFR₁ in the presence of increasing concentrations of Compound 10. The inflection point on the curve shows the concentration of Compound 10 at which it increases FGFR₁ phosphorylation. The data indicates that Compound 10 augmented the effects of FGF-2.

### Example 4. Stroke Recovery in vivo (Compound 10 Given on Day 1, 2, and 3 After Stroke)

Compound 10 was tested for its effectiveness in a rodent model of stroke recovery. Twenty male Sprague Dawley Rats (Charles River Laboratories) each weighing 300-400 g were used in this experiment. First, anesthesia was induced in an induction chamber with 2-3% isoflurane in N₂O:O₂ (2:1) and maintained with 1-1.5% isoflurane via face mask. Adequate depth of anesthesia was assessed by lack of withdrawal to hindlimb pinch and loss of eyeblink reflex. Once anesthetized, animals received cefazolin sodium (40 mg/kg, i.p.) and buprenorphine SR (0.9-1 mg/kg, s.c.). Cefazolin was used as a prophylactic antibiotic. A veterinary ophthalmic ointment (Sodium Chloride hypertonicity ophthalmic ointment (Muro 128 Sterile Ophthalmic 5% Ointment)) was applied to the eyes.

A small focal stroke (infarct) was made on the right side of the surface of the brain (cerebral cortex) by middle cerebral artery occlusion (MCAO). The stroke becomes fixed in size and location within 24 hours after the MCAO. The stroke results in impaired sensorimotor function of the contralateral (left) limbs that recover slowly and incompletely over time.

For stroke surgery, the right side of the head was shaved with electric clippers (patch of approximately 3 cm by 5 cm between eye and ear). The region was carefully cleaned with Hibiclens and alcohol. Using aseptic technique, an incision was made midway between the eye and eardrum canal.

The temporalis muscle was isolated, bisected, and reflected. A small window of bone was removed via drill and rongeurs (subtemporal craniectomy) to expose the MCA. Care was taken not to remove the zygomatic arch or to transect the facial nerve that would impair the ability of the animal to chew after surgery. Using a dissecting microscope, the dura was incised, and the MCA was electrocoagulated from just proximal to the olfactory tract to the inferior cerebral vein (taking care not to rupture this vein), using microbipolar electrocauterization. The MCA was then transected. The temporalis muscle was then repositioned, and the incision was closed subcutaneously with sutures. The skin incision was closed with surgical staples (2-3 required). Throughout the procedure, body temperature was maintained at 37.0°±1° C. using a self-regulating heating pad connected to a rectal thermometer. Following surgery, animals remained on a heating pad until they woke up from anesthesia. They were returned to clean home cages. The animals were housed 2 per cage before and after surgery, unless severe aggression was displayed, or death of cage mate(s). They were observed frequently on the day of MCAO surgery (Day 0) and at least once daily thereafter.

The rats were randomly assigned into two groups of ten each. Each group was injected intravenously (i.v.) with 2 ml/kg Compound 10 at 10 mg/kg or vehicle (18% Cremophor RH40 and 10% DMSO in 5% dextrose solution (D5W)) on Day 1, 2, and 3 after MCAO. Day 0 is the day of the MCAO, and the days after the MCAO are numbered consecutively (Day 1, Day 2, Day 3, etc.) D-pre represents the day prior to the MCAO.

Behavioral evaluations of sensorimotor function were done by investigators blinded to treatment assignment. Limb placing tests were done on Day Pre (one day pre-MCAO operation), Day 1, Day 3, Day 4, Day 7, Day 14, and Day 21. The limb placing tests were divided into forelimb and hindlimb tests. For the forelimb-placing test, the examiner held the rat close to a tabletop and scored the rat's ability to place the forelimb on the tabletop in response to whisker, visual, tactile, or proprioceptive stimulation. Similarly, for the hindlimb placing test, the examiner assessed the rat's ability to place the hindlimb on the tabletop in response to tactile and proprioceptive stimulation. Separate sub-scores were obtained for each mode of sensory input and added to give total scores (for the forelimb placing test: 0=normal, 12=maximally impaired; for the hindlimb placing test: 0=normal; 6=maximally impaired). Scores were given in half-point increments (see below).
Forelimb placing test (0-12):
   whisker placing (0-2);
   visual placing (forward (0-2), sideways (0-2))
   tactile placing (dorsal (0-2), lateral (0-2))
   proprioceptive placing (0-2).
Hindlimb placing test (0-6):
   tactile placing (dorsal (0-2), lateral (0-2))
   proprioceptive placing (0-2).
   For each subtest, animals are scored as followed:
      0.0=immediate response
      0.5=response within 2 seconds
      1.0=response of 2-3 seconds
      1.5=response of >3 seconds
      2.0=no response

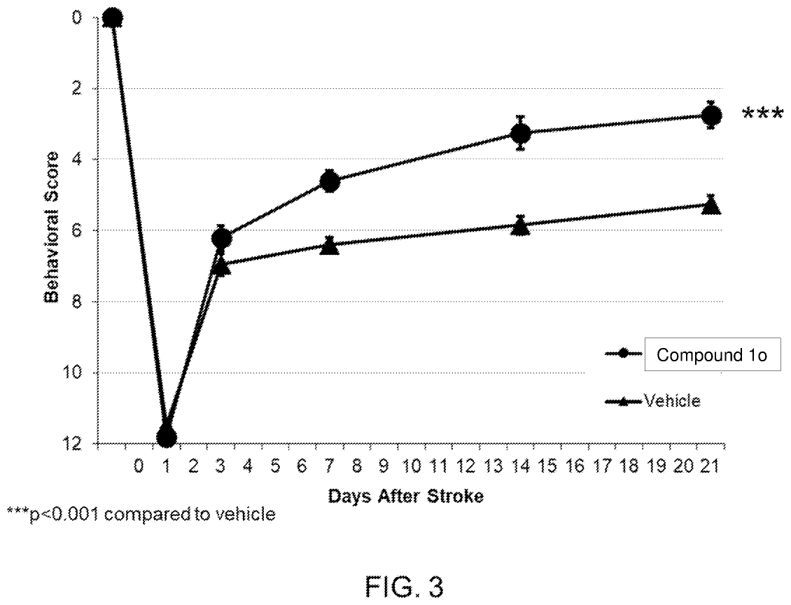

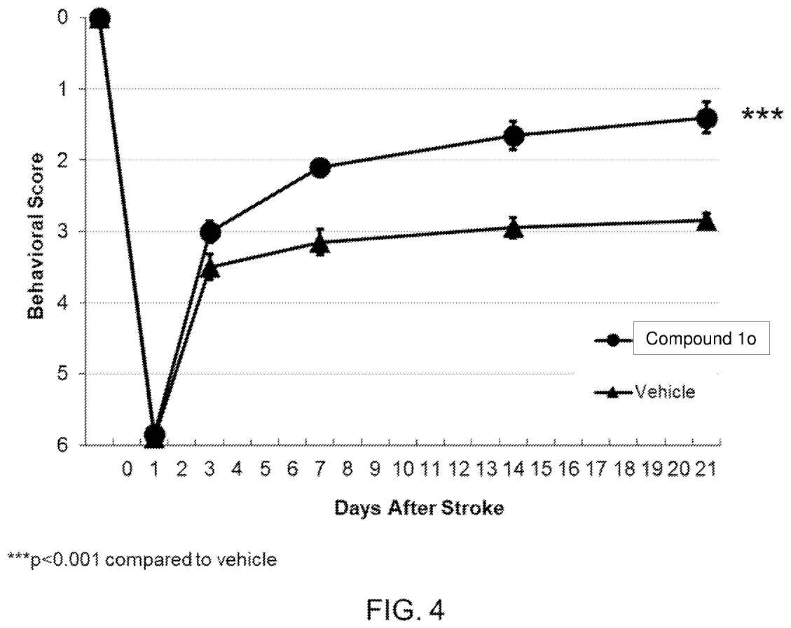

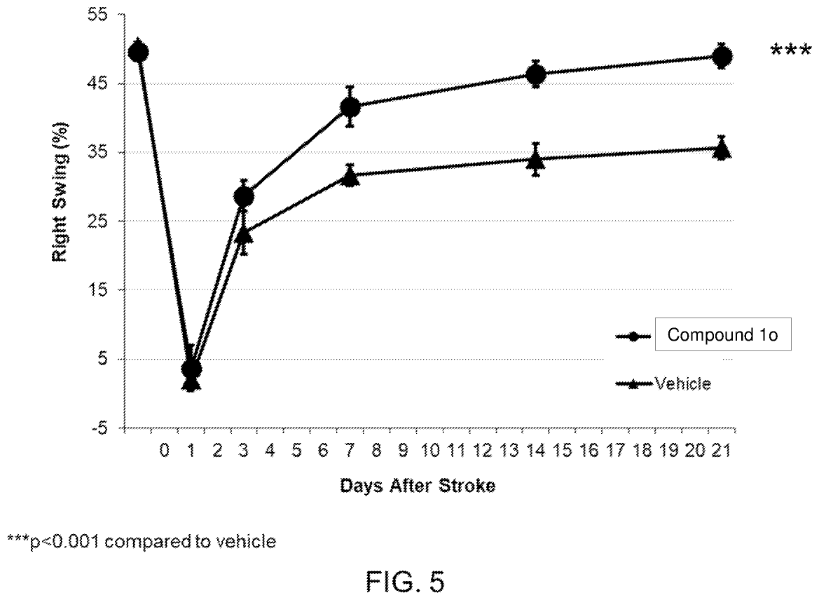

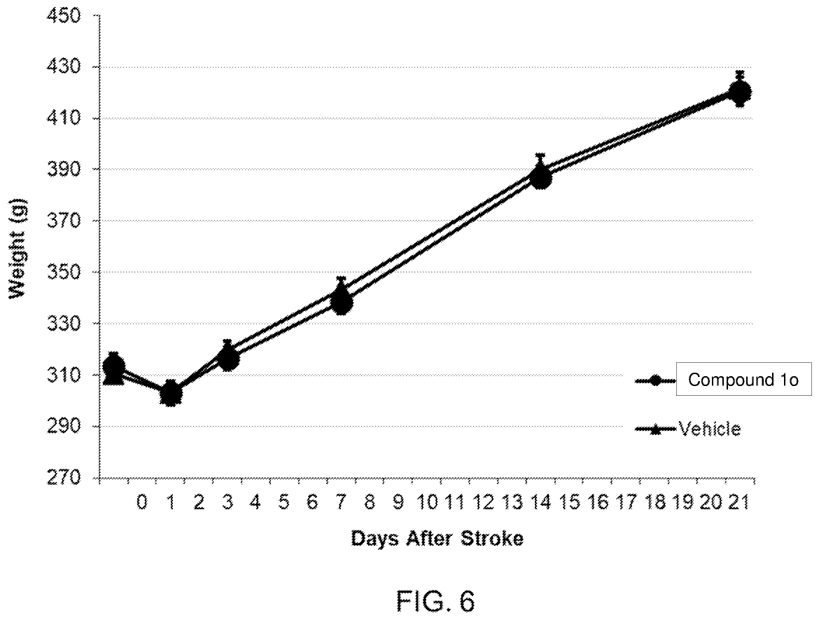

The results from limb placing tests, body swing tests, and body weight pre- and post-MCAO are shown in FIGS. 3-6.

Typically, after an initial rapid rise, there is a continued slow, steady, and partial improvement in sensorimotor function (as measured by forelimb and hindlimb placing and body swing tests) during the first three weeks after stroke. Previous studies indicate that recovery plateaus at this time and does not change thereafter. Animals treated with Compound 10 showed a clear and significant augmentation of sensorimotor recovery on all three measures compared to vehicle-treated animals (p<0.001 by two-way repeated-measures ANOVA). The normal rise in body weight following surgery was not affected by treatment with Compound 10.

Treatment with Compound 10 was initiated at one day after stroke, at a time when infarct size and location is fixed. This indicates that Compound 10 does not promote enhanced recovery by reduction of infarct size, but rather through a separate recovery-promoting mechanism.

### Example 5. Anti-Coronavirus Activity

Compound 10 was evaluated for its ability to reduce human coronavirus 229E induced cellular toxicity in HAP1 cells with and without the addition of a low concentration of FGF-2. HAP1 cells were seeded at a density of 1×10⁴ cells/well in a volume of 100 μL in DMEM supplemented with 10% FBS. Following a 24-hour incubation at 37° C./5% CO₂ the cells were pre-incubated with and without (media only) exogenous FGF-2 (1 ng/ml) and Compound 10 (0.002 μM, 0.008 μM, 0.04 μM, 0.2 μM, or 1 μM; plated in triplicate) for 24 hours prior (D-1) to the addition of human coronavirus 229E at a pre-determined titer. On the day of viral infection (D0), and one and two days thereafter (D1 and D2), freshly prepared FGF-2 and Compound 10 were added. The cultures were incubated for 4 days at 37° C./5% CO₂, after which the cells were stained for cell survival with the tetrazolium dye XTT. Compound 10 and FGF-2 had no effect on cell survival in the absence of the virus.

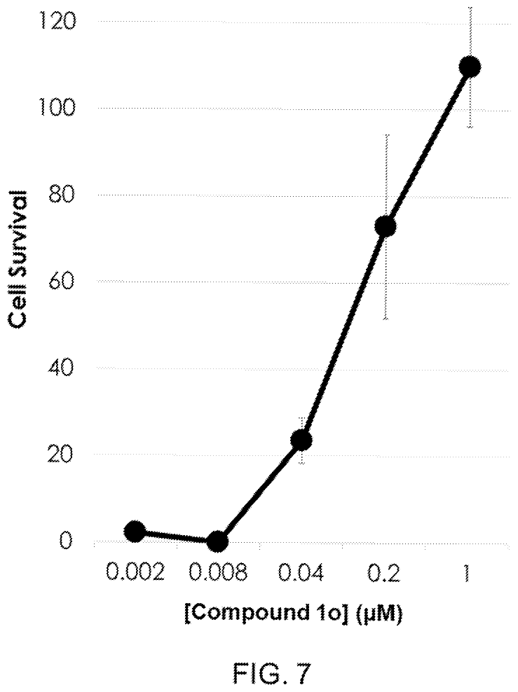

As shown in FIG. 7, the combination of FGF-2 and Compound 10 increases cell survival in HAP1 cells infected with human coronavirus 229E.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of treating a subject having a disease or injury comprising administering to the subject a therapeutically effective amount of a compound, wherein the compound is a compound of formula (I): 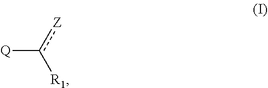
or a pharmaceutically acceptable salt or a tautomer thereof, wherein
Q is optionally substituted C₆-C₁₀ aryl or optionally substituted 6- to 10-membered heterocyclyl;
R₁ is H, OH, optionally substituted C₁-C₆ alkyl, optionally substituted C₆-C₁₆ aryl, or optionally substituted 6- to 12-membered heteroaryl; and
Z is O or NR_{c} and [symbol] is a double bond,
wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein Rₐ and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, form an optionally substituted 6- to 10-membered heterocyclyl, or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol]
is a single bond, and R₁ and Z, together with the carbon atom to which they are attached, form an optionally substituted oxazolidinyl or optionally substituted thiazolidinyl; or [symbol]
is a single bond, and Z is OH.

2. The method of claim 1, wherein the disease or injury is stroke; congenital hypogonadotropic hypogonadism; cerebral hemorrhage; traumatic brain injury (TBI); spinal cord injury (SCI); peripheral vascular disease (PVD); wounds; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection.

3. The method of claim 2, wherein the disease or injury is stroke, provided that:
when Q is optionally substituted C₆-C₁₀ aryl, Rᵢ is H, Z is NR_{c}, and R_{c} is NR_{f}R_{g}, R_{f} and R_{g}, together with the nitrogen atom to which they are attached, do not form optionally substituted piperazinyl;
when Z is NR_{c}, and R_{c} is NR_{f}R_{g}, one of R_{f} and R_{g} is H, and the other of R_{f} and R_{g} is C₁-C₆ alkyl substituted with one oxo, R_{g} is not further substituted with unsaturated heterocyclyl; piperazinyl; aryl; oxo;
OR^{k}, wherein Rₖ is aryl or heterocyclyl; or NHR_{I}, wherein Rᵢ is aryl, cycloalkyl, or alkyl substituted with oxo; and
when Q is optionally substituted C₆-C₁₀ aryl and Z is O, R₁ not C₁-C₆ alkyl substituted with NHRₘ, wherein Rₘ is aryl.

4. The method of claim 3, wherein the stroke is acute stroke.

5. The method of claim 3, wherein the stroke is in a recovery phase.

6. The method of claim 2, wherein the disease or injury is congenital hypogonadotropic hypogonadism.

7. The method of claim 6, wherein the congenital hypogonadotropic hypogonadism is Kallmann Syndrome.

8. The method of claim 2, wherein the disease or injury is viral infection.

9. A method of increasing spermatogenesis in a subject comprising administering to a subject a therapeutically effective amount of a compound, wherein the compound is a compound of formula (I): 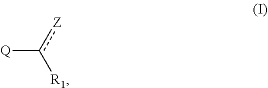
or a pharmaceutically acceptable salt or a tautomer thereof, wherein
Q is optionally substituted C₆-C₁₀ aryl or optionally substituted 6- to 10-membered heterocyclyl;
R₁ is H, OH, optionally substituted C₁-C₆ alkyl, optionally substituted C₆-C₁₆ aryl, or optionally substituted 6- to 12-membered heteroaryl; and
Z is O or NR_{c} and [symbol] is a double bond,
wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein Rₐ and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, form an optionally substituted 6- to 10-membered heterocyclyl, or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol]
is a single bond, and R₁ and Z, together with the carbon atom to which they are attached, form an optionally substituted oxazolidinyl or optionally substituted thiazolidinyl; or [symbol]
is a single bond, and Z is OH.

10. The method of any one of claims 1-9, wherein the compound is a compound of formula (la): 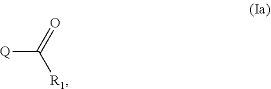
or a pharmaceutically acceptable salt thereof.

11. The method of claim 10, wherein R₁ is H.

12. The method of claim 10, wherein R₁ is C₁-C₆ alkyl.

13. The method of claim 10, wherein R₁ is optionally substituted C₆-₁₆ aryl.

14. The method of claim 13, wherein R₁ is optionally substituted phenyl.

15. The method of claim 13, wherein R₁ is

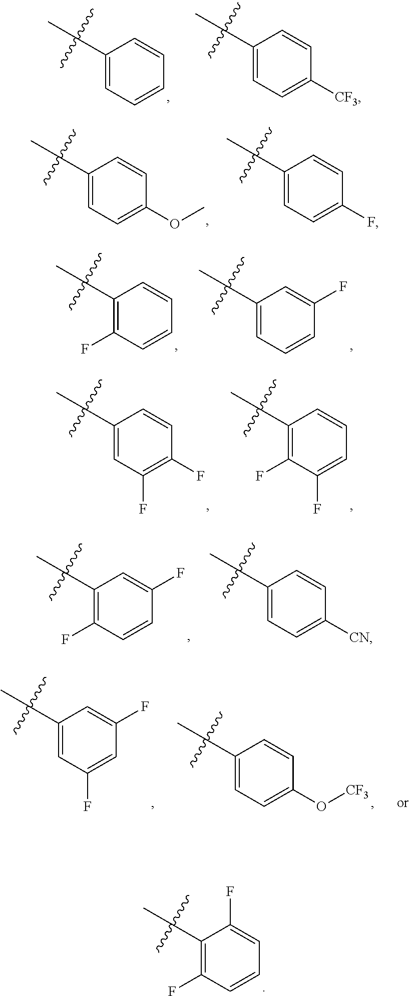

16. The method of claim 10, wherein R₁ is optionally substituted 6- to 12-membered heteroaryl.

17. The method of claim 16, wherein R₁ is

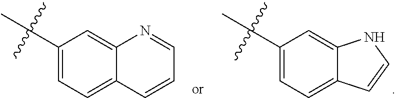 [SMILES: *c1ccc2cccnc2c1 ; *c1ccc2cc[nH]c2c1]

18. The method of any one of claims 1-9, wherein the compound is a compound of formula (Ib): 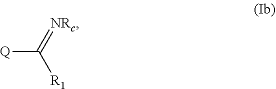
or a pharmaceutically acceptable salt or a tautomer thereof.

19. The method of claim 18, wherein R₁ is H.

20. The method of claim 18 or 19, wherein R_{c} is OR_{d}.

21. The method of claim 20, wherein R_{c} is OH.

22. The method of claim 18 or 19, wherein R_{c} is optionally substituted C₁-C₆ alkyl.

23. The method of claim 22, wherein R_{c} is methyl substituted with one or two optionally substituted C₆-C₁₆ aryl or C₁-C₁₅ heterocyclyl.

24. The method of claim 16, wherein R_{c} is

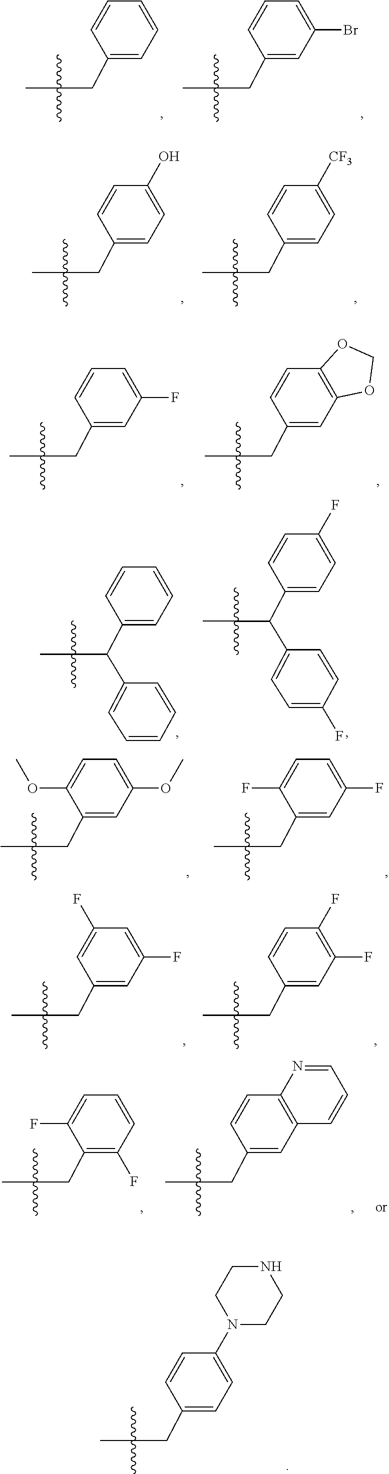

25. The method of claim 18 or 19, wherein R_{c} is optionally substituted C₆-C₁₆ aryl.

26. The method of claim 25, wherein R_{c} is

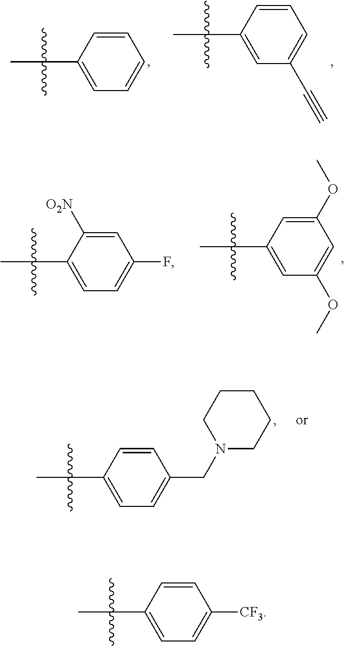

27. The method of claim 18 or 19, wherein R_{c} is optionally substituted C₁-C₁₅ heterocyclyl.

28. The method of claim 27, wherein R_{c} is

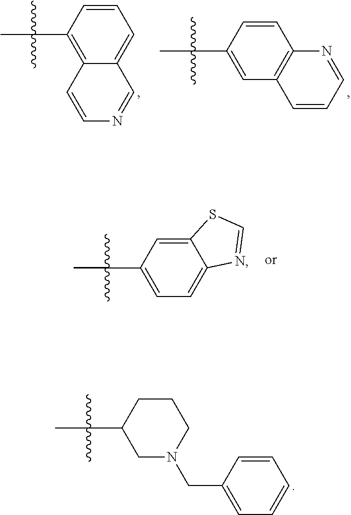 [SMILES: *c1cccc2cnccc12 ; *c1ccc2ncccc2c1 ; *c1ccc2ncsc2c1 ; "or" ; *C1CCCN(Cc2ccccc2)C1]

29. The method of claim 18 or 19, wherein R_{c} is optionally substituted C₄-C₁₃ cycloalkenyl.

30. The method of claim 29, wherein R_{c} is

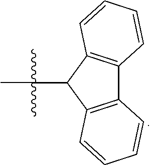 [SMILES: *C1c2ccccc2-c2ccccc21]

31. The method of claim 18 or 19, wherein R_{c} is NR_{f}R_{g}.

32. The method of claim 31, wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl.

33. The method of claim 32, wherein R_{c} is NH₂.

34. The method of claim 31, wherein R_{f} and R_{g} are independently H or optionally substituted C₈-C₁₈ aryl, wherein at least one of R_{f} and R_{g} is optionally substituted C₆-C₁₆ aryl.

35. The method of claim 34, wherein R_{c} is

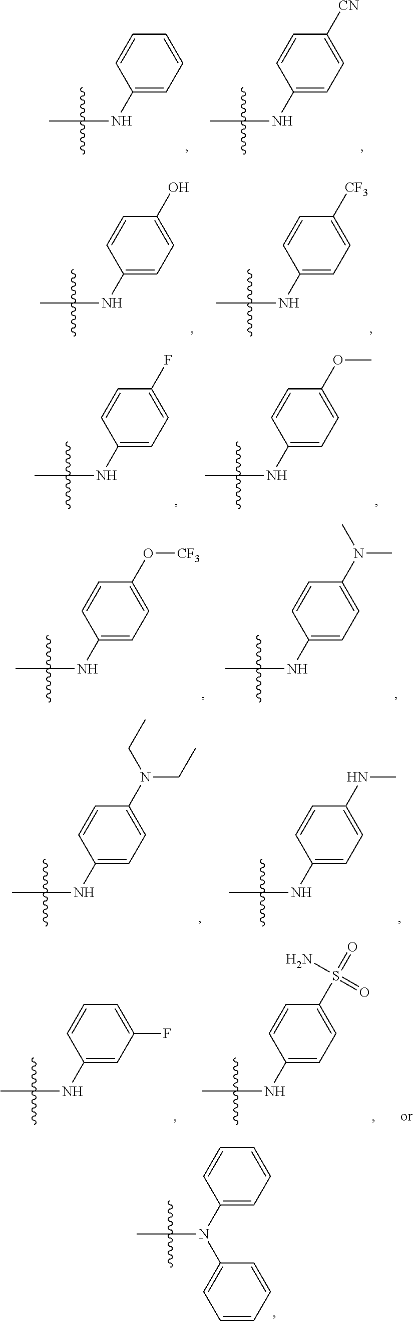

36. The method of claim 31, wherein R_{f} and R_{g} are independently H or optionally substituted C₁-C₆ alkyl, wherein at least one of R_{f} and R_{g} is optionally substituted C₁-C₆ alkyl.

37. The method of claim 36, the compound is a compound of formula (Ib-2): 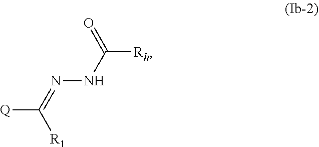
or a pharmaceutically acceptable salt thereof, wherein Rₕ is optionally substituted C₁-C₆ alkyl, optionally substituted C₃-C₈ cycloalkyl, optionally substituted C₆-C₁₆ aryl, or optionally substituted C₁-C₁₅ heterocyclyl.

38. The method of claim 37, wherein Rₕ is optionally substituted C₁-C₆ alkyl.

39. The method of claim 38, wherein Rₕ is CH₂N(CH₃)₂.

40. The method of claim 37, wherein Rₕ is optionally substituted C₃-C₈ cycloalkyl.

41. The method of claim 40, wherein Rₕ is

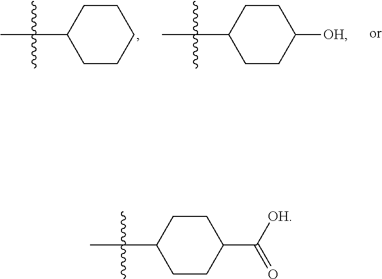 [SMILES: *C1CCCCC1 ; *C1CCC(O)CC1 ; "or" ; *C1CCC(C(=O)O)CC1]

42. The method of claim 37, wherein Rₕ is optionally substituted C₆-C₁₄ aryl.

43. The method of claim 42, wherein Rₕ is

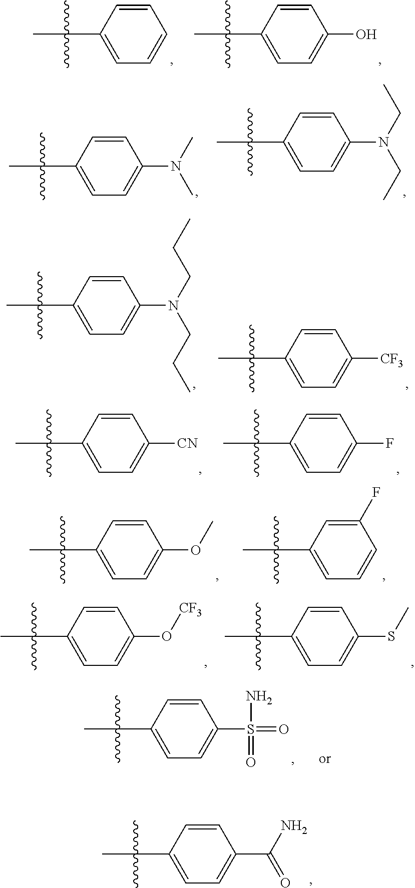

44. The method of claim 37, wherein Rₕ is optionally substituted C₁-C₁₅ heterocyclyl.

45. The method of claim 44, wherein Rₕ is

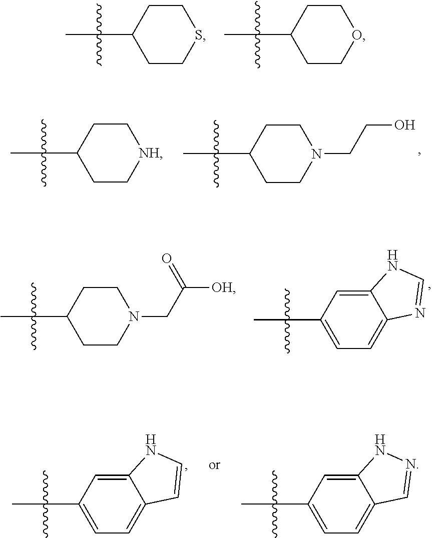

46. The method of claim 31, wherein R_{f} and R_{g} are independently H or optionally substituted C₃-C₈ cycloalkyl, wherein at least one of R_{f} and R_{g} is optionally substituted C₃-C₈ cycloalkyl.

47. The method of claim 46, wherein R_{c} is

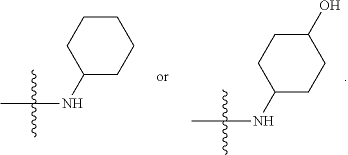 [SMILES: *NC1CCCCC1 ; *NC1CCC(O)CC1]

48. The method of claim 31, wherein R_{f} and R_{g} are independently H or optionally substituted C₁-C₁₅ heterocyclyl, wherein at least one of R_{f} and R_{g} is optionally substituted C₁-C₁₅ heterocyclyl.

49. The method of claim 48, wherein R_{c} is

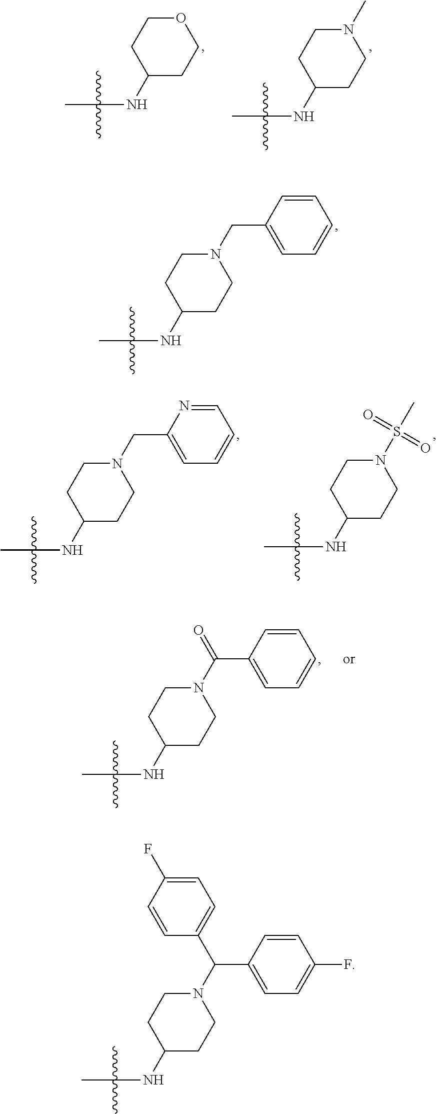

50. The method of claim 31, wherein R_{f} and R_{g}, together with the nitrogen atom to which they are attached, forms an optionally substituted 6- to 10-membered heterocyclyl.

51. The method of claim 50, wherein R_{c} is

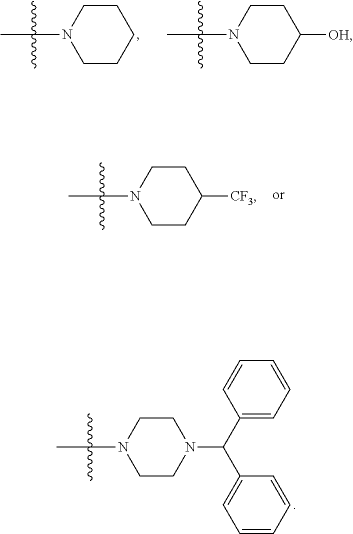 [SMILES: *N1CCCCC1 ; *N1CCC(O)CC1 ; *N1CCC(C(F)(F)F)CC1 ; "or" ; *N1CCN(C(c2ccccc2)c2ccccc2)CC1]

52. The method of claim 18 or 19, wherein R_{c} is N═C(R₁′)Q′.

53. The method of claim 52, wherein R₁′ is H.

54. The method of claim 52 or 53, wherein Q′ and Q are identical.

55. The method of any one of claims 1 to 9, wherein [symbol] is a single bond, and Rᵢ and Z, together with the carbon atom to which they are attached, form an optionally substituted oxazolidinyl or optionally substituted thiazolidinyl.

56. The method of claim 55, wherein R₁ and Z, together with the carbon atom to which they are attached, form an optionally substituted thiazolidinyl.

57. The method of claim 56, wherein R₁ and Z, together with the carbon atom to which they are attached, form

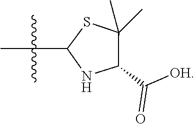 [SMILES: *C1N[C@@H](C(=O)O)C(C)(C)S1]

58. The method of any one of claims 1 to 57, wherein Q is 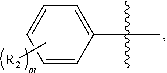
wherein each R₂ is independently halo or NRₐR_{b}, wherein Rₐ and R_{b} are independently H; optionally substituted C₁-C₆ alkyl; optionally substituted C₈-C₁₈ aryl; or SO₂Rᵢ, wherein Rᵢ is H or C₁-C₆ alkyl; or Rₐ and R_{b}, together with the nitrogen atom to which they are attached, forms an optionally substituted 5- to 10-membered heterocyclyl; and m is 0 to 5.

59. The method of claim 58, wherein m is 0.

60. The method of claim 58, wherein m is 1.

61. The method of claim 60, wherein Q is

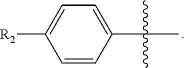

62. The method of claim 60, wherein Q is

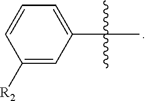

63. The method of claim 60, wherein Q is

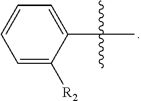

64. The method of any one of claims 60 to 63, wherein R₂ is halo.

65. The method of any one of claims 60 to 63, wherein R₂ is NRₐR_{b}.

66. The method of claim 65, wherein Rₐ and R_{b} are independently H or optionally substituted C₁-C₆ alkyl.

67. The method of claim 66, wherein R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂.

68. The method of claim 67, wherein R₂ is N(CH₂CH₃)₂.

69. The method of claim 65, wherein Rₐ and R_{b}, together with the nitrogen atom to which they are attached, forms an optionally substituted 5- to 10-membered heterocyclyl.

70. The method of claim 69, wherein R₂ is

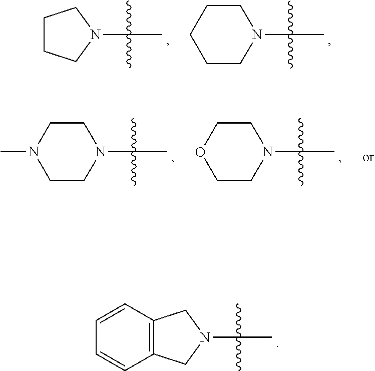

71. The method of claim 65, wherein Rₐ and R_{b} are independently H or optionally substituted c₆-C₁₆ aryl.

72. The method of claim 71, wherein R₂ is

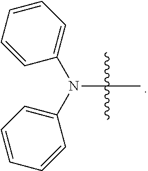 [SMILES: *N(c1ccccc1)c1ccccc1]

73. The method of claim 65, wherein R₂ is NH(SO₂CH₃).

74. The method of claim 58, wherein m is 2.

75. The method of claim 74, wherein Q is

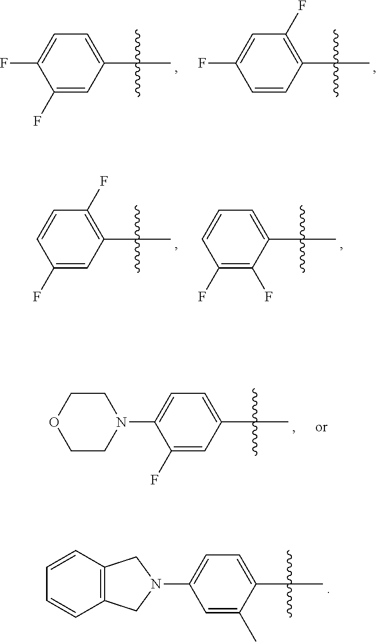 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1 ; *c1ccc(N2Cc3ccccc3C2)cc1C]

76. The method of any one of claims 1-57, wherein Q is optionally substituted 6- to 10-membered heterocyclyl.

77. The method of claim 76, wherein Q is

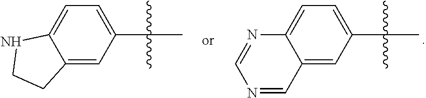 [SMILES: *c1ccc2c(c1)CCN2 ; *c1ccc2ncncc2c1]

78. The method of any one of claims 1 to 9, wherein the compound is: 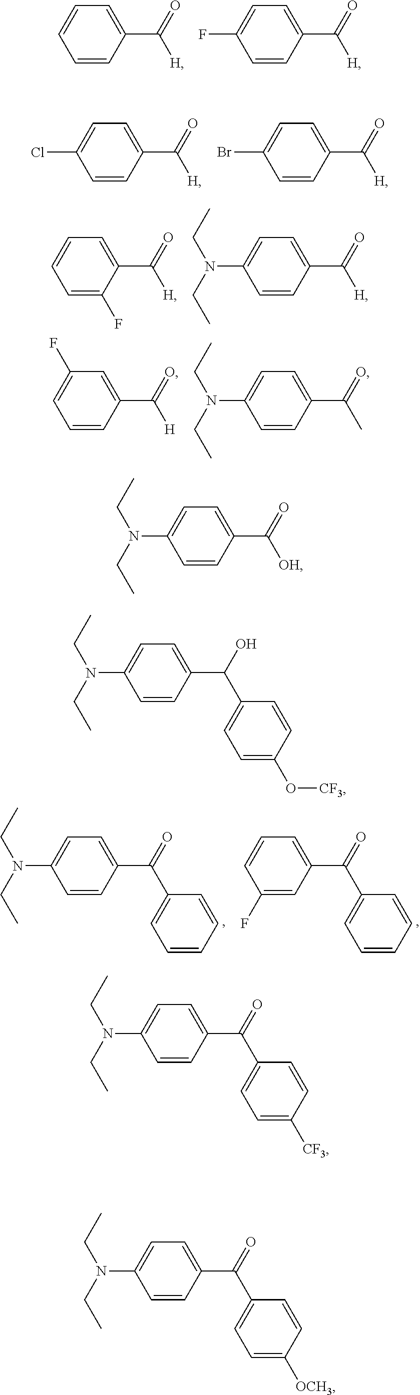 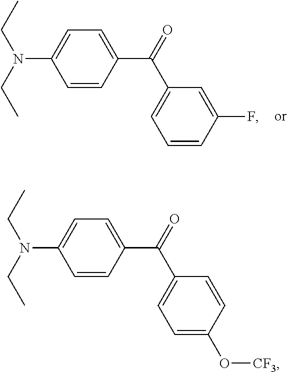 [SMILES: CCN(CC)c1ccc(C(=O)c2cccc(F)c2)cc1 ; CCN(CC)c1ccc(C(=O)c2ccc(OC(F)(F)F)cc2)cc1]
or a pharmaceutically acceptable salt thereof.

79. The method of any one of claims 1 to 9, wherein the compound is: 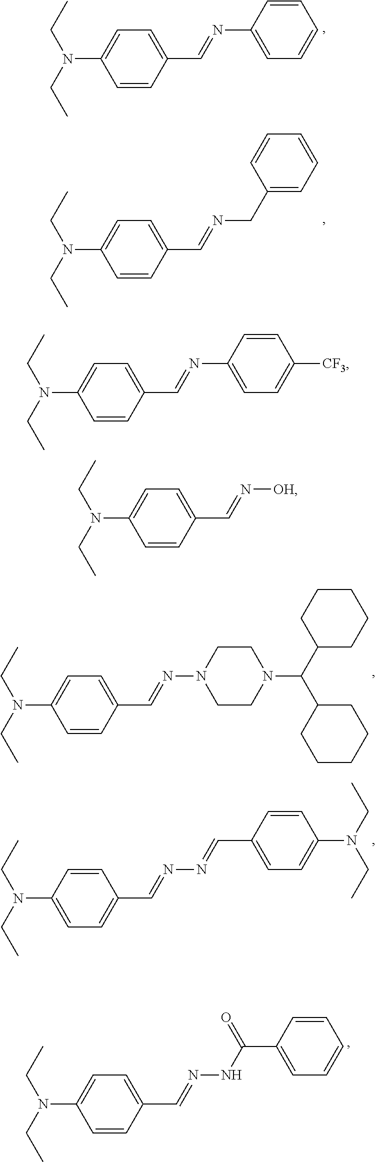 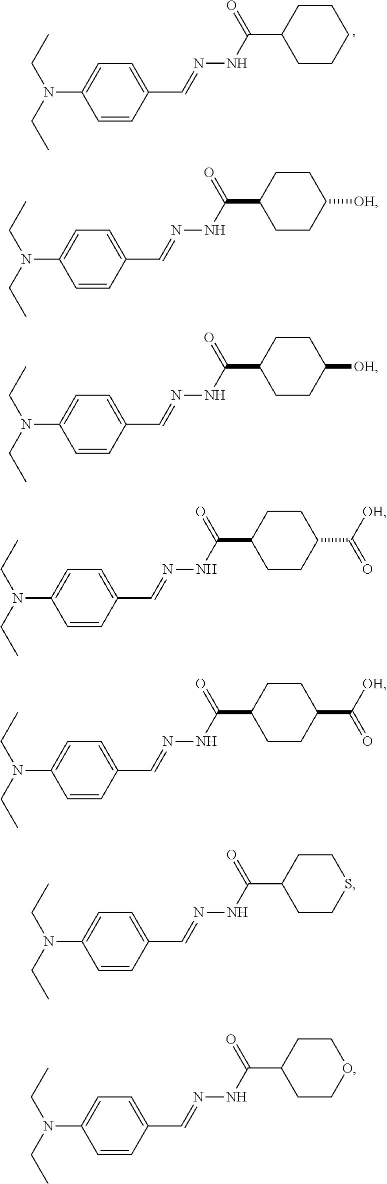 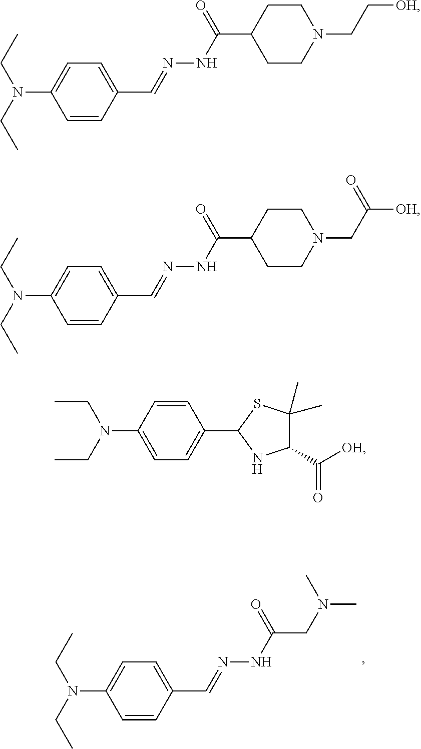 [SMILES: CCN(CC)c1ccc(/C=N/NC(=O)C2CCN(CCO)CC2)cc1 ; CCN(CC)c1ccc(/C=N/NC(=O)C2CCN(CC(=O)O)CC2)cc1 ; CCN(CC)c1ccc(C2N[C@@H](C(=O)O)C(C)(C)S2)cc1 ; CCN(CC)c1ccc(/C=N/NC(=O)CN(C)C)cc1] 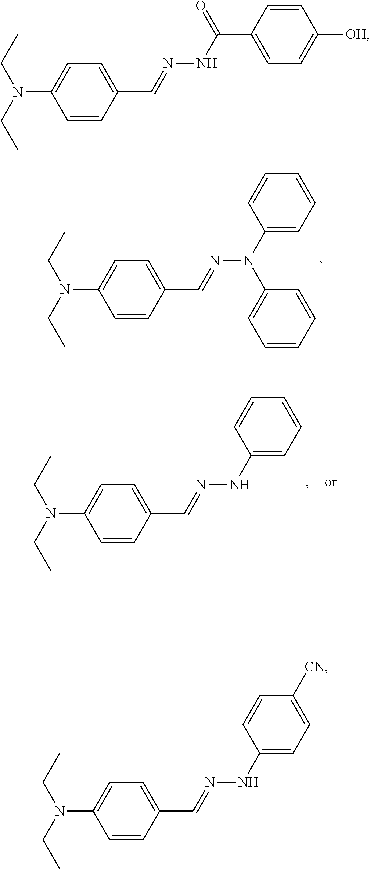 [SMILES: CCN(CC)c1ccc(/C=N/NC(=O)c2ccc(O)cc2)cc1 ; CCN(CC)c1ccc(/C=N/N(c2ccccc2)c2ccccc2)cc1 ; CCN(CC)c1ccc(/C=N/Nc2ccccc2)cc1 ; CCN(CC)c1ccc(/C=N/Nc2ccc(C#N)cc2)cc1]
or a pharmaceutically acceptable salt thereof.

80. A compound of formula (I′): 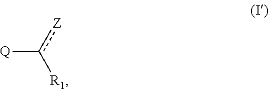
or a pharmaceutically acceptable salt or a tautomer thereof, wherein
Q is optionally substituted C₆-C₁₀ aryl or optionally substituted 6- to 10-membered heterocyclyl;
R₁ is H; and
Z is NR_{c} and [symbol] is a double bond,
wherein R_{c} is a group of formula: 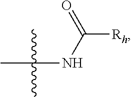
wherein Rₕ is substituted C₃-C₈ cycloalkyl or optionally substituted C₁-C₁₅ heterocyclyl; or
R_{c} is a group of formula N═C(R₁′)Q′, wherein R₁′ is H and Q′ is optionally substituted C₆-C₁₀ aryl or optionally substituted 6- to 10-membered heterocyclyl; or
R_{c} is a group of formula: 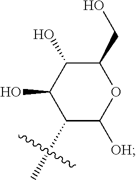
or [symbol]
is a single bond, and R₁ and Z, together with the carbon atom to which they are attached, form an optionally substituted oxazolidinyl or optionally substituted thiazolidinyl.

81. The compound of claim 80, wherein the compound is a compound of formula (Ib′): 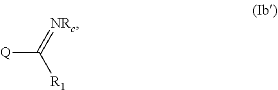
or a pharmaceutically acceptable salt or a tautomer thereof.

82. The compound of claim 81, wherein the compound is a compound of formula (Ib′-2): 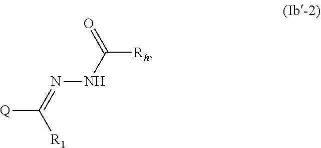
or a pharmaceutically acceptable salt thereof.

83. The compound of claim 82, wherein Rₕ is C₃-C₈ cycloalkyl having at least one substituent.

84. The compound of claim 83, wherein Rₕ is

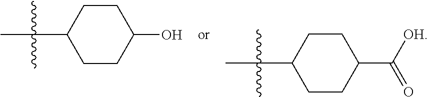 [SMILES: *C1CCC(O)CC1 ; *C1CCC(C(=O)O)CC1]

85. The compound of claim 82, wherein Rₕ is optionally substituted C₁-C₁₅ heterocyclyl.

86. The compound of claim 85, wherein Rₕ is

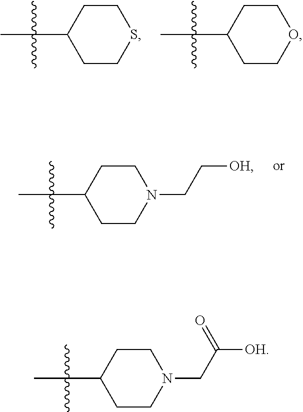 [SMILES: *C1CCSCC1 ; *C1CCOCC1 ; *C1CCN(CCO)CC1 ; "or" ; *C1CCN(CC(=O)O)CC1]

87. The compound of claim 81, wherein R_{c} is N═C(R₁′)Q′.

88. The compound of claim 87, wherein Q′ and Q are identical.

89. The compound of claim 80, wherein Rᵢ and Z, together with the carbon atom to which they are attached, form an optionally substituted oxazolidinyl or optionally substituted thiazolidinyl.

90. The compound of claim 89, wherein R₁ and Z, together with the carbon atom to which they are attached, form an optionally substituted thiazolidinyl.

91. The compound of claim 90, wherein R₁ and Z, together with the carbon atom to which they are attached, form

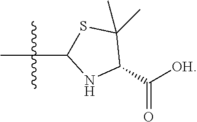 [SMILES: *C1N[C@@H](C(=O)O)C(C)(C)S1]

92. The compound of any one of claims 80 to 91, wherein Q is 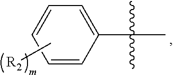
wherein each R₂ is independently halo or NRₐR_{b}, wherein Rₐ and R_{b} are independently H; optionally substituted C₁-C₆ alkyl; optionally substituted C₆-C₁₆ aryl; or SO₂Rᵢ, wherein Rᵢ is H or C₁-C₆ alkyl; or Rₐ and R_{b}, together with the nitrogen atom to which they are attached, form an optionally substituted 5- to 10-membered heterocyclyl; and m is 0 to 5.

93. The compound of claim 92, wherein m is 0.

94. The compound of claim 92, wherein m is 1.

95. The compound of claim 94, wherein Q is

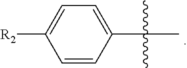

96. The compound of claim 94, wherein Q is

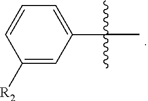

97. The compound of claim 94, wherein Q is

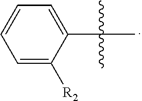

98. The compound of any one of claims 94 to 97, wherein R₂ is halo.

99. The compound of any one of claims 94 to 97, wherein Rz is NRₐR_{b}.

100. The compound of claim 99, wherein Rₐ and R_{b} are independently H or optionally substituted C₁-C₆ alkyl.

101. The compound of claim 100, wherein R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂.

102. The compound of claim 101, wherein R₂ is N(CH₂CH₃)₂.

103. The compound of claim 99, wherein Rₐ and R_{b}, together with the nitrogen atom to which they are attached, form an optionally substituted 5- to 10-membered heterocyclyl.

104. The compound of claim 103, wherein R₂ is

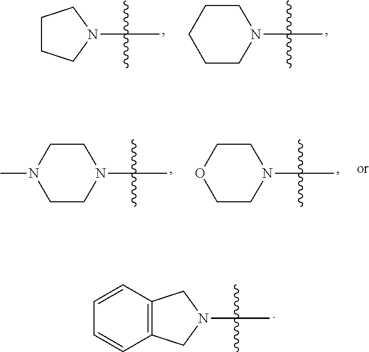

105. The compound of claim 99, wherein Rₐ and R_{b} are independently H or optionally substituted C₆-C₁₆ aryl.

106. The compound of claim 105, wherein R₂ is

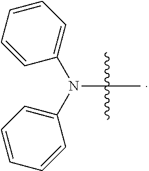 [SMILES: *N(c1ccccc1)c1ccccc1]

107. The compound of claim 99, wherein R₂ is NH(SO₂CH₃).

108. The compound of claim 92, wherein m is 2.

109. The compound of claim 108, wherein Q is

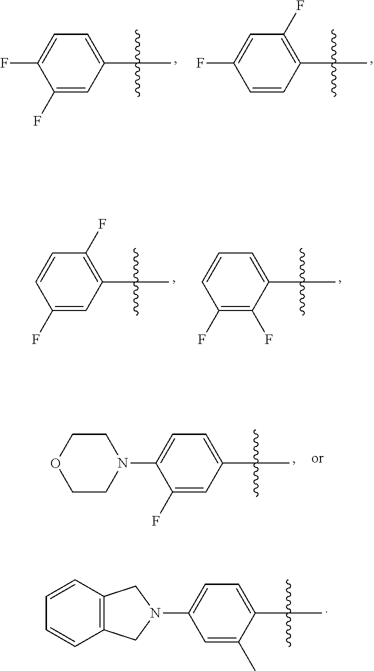 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1 ; *c1ccc(N2Cc3ccccc3C2)cc1C]

110. The compound of any one of claims 80-91, wherein Q is optionally substituted 6- to 10-membered heterocyclyl.

111. The compound of claim 110, wherein Q is

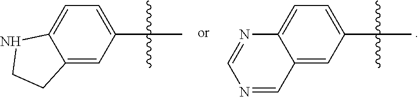 [SMILES: *c1ccc2c(c1)CCN2 ; *c1ccc2ncncc2c1]

112. The compound of claim 80, wherein the compound is: 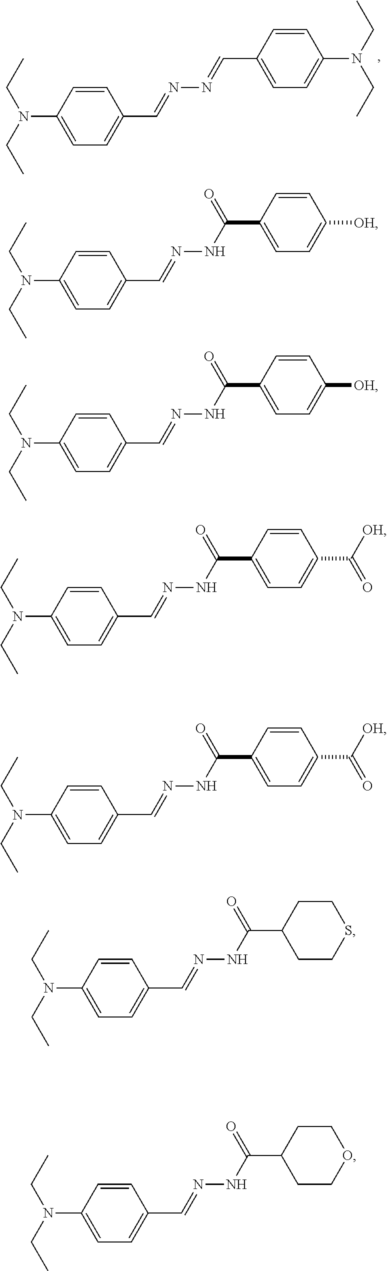 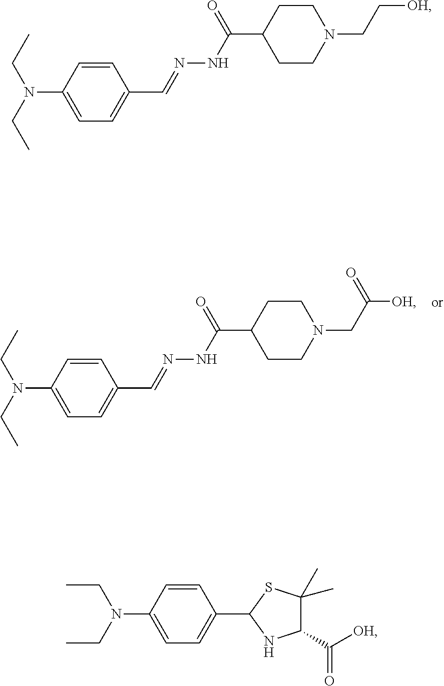 [SMILES: CCN(CC)c1ccc(/C=N/NC(=O)C2CCN(CCO)CC2)cc1 ; CCN(CC)c1ccc(/C=N/NC(=O)C2CCN(CC(=O)O)CC2)cc1 ; CCN(CC)c1ccc(C2N[C@@H](C(=O)O)C(C)(C)S2)cc1]
or a pharmaceutically acceptable salt thereof.

113. A pharmaceutical composition comprising a compound of any one of claims 80 to 112 or a pharmaceutically acceptable salt thereof, and a pharmaceutically acceptable excipient.

114. A pharmaceutical composition comprising a compound of formula (I): 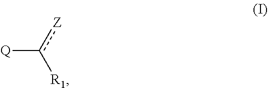
wherein
Q is optionally substituted C₆-C₁₀ aryl, or optionally substituted 6- to 10-membered heterocyclyl;
R₁ is H, OH, optionally substituted C₁-C₆ alkyl, optionally substituted C₆-C₁₆ aryl or optionally substituted 6- to 12-membered heteroaryl; and
Z is O or NR_{c}, and [symbol] is a double bond,
wherein R_{c} is H; optionally substituted C₁-C₆ alkyl; optionally substituted C₂-C₆ alkenyl; optionally substituted C₂-C₆ alkynyl; optionally substituted C₃-C₈ cycloalkyl; optionally substituted C₄-C₁₃ cycloalkenyl; optionally substituted C₁-C₁₅ heterocyclyl; optionally substituted C₆-C₁₆ aryl; OR_{d}; SRₑ; or NR_{f}R_{g}, wherein Rₐ and Rₑ are independently H or C₁-C₆ alkyl and wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₆-C₁₆ aryl, or R_{f} and R_{g}, together with the nitrogen atom to which they are attached, forms an optionally substituted 6- to 10-membered heterocyclyl, or or R_{f} and R_{g}, together with the nitrogen atom to which they are attached, form N═C(R₁′)Q′, wherein R₁′ is H, OH, optionally substituted C₁-C₆ alkyl, optionally substituted C₆-C₁₆ aryl, or optionally substituted 6- to 12-membered heteroaryl and Q′ is optionally substituted C₆-C₁₀ aryl or optionally substituted 6- to 10-membered heterocyclyl; or [symbol]
is a single bond, and R₁ and Z, together with the carbon atom to which they are attached, form an optionally substituted oxazolidinyl or optionally substituted thiazolidinyl,
or a pharmaceutically acceptable salt or a tautomer thereof, and a pharmaceutically acceptable excipient.

115. The pharmaceutical composition of claim 114, wherein the compound is a compound of formula (la): 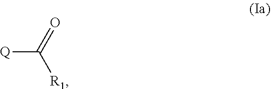
or a pharmaceutically acceptable salt thereof.

116. The pharmaceutical composition of claim 115, wherein R₁ is H.

117. The pharmaceutical composition of claim 115, wherein R₁ is C₁-C₆ alkyl.

118. The pharmaceutical composition of claim 115, wherein R₁ is optionally substituted C₈-16 aryl

119. The pharmaceutical composition of claim 118, wherein R₁ is optionally substituted phenyl.

120. The pharmaceutical composition of claim 119, wherein R₁ is

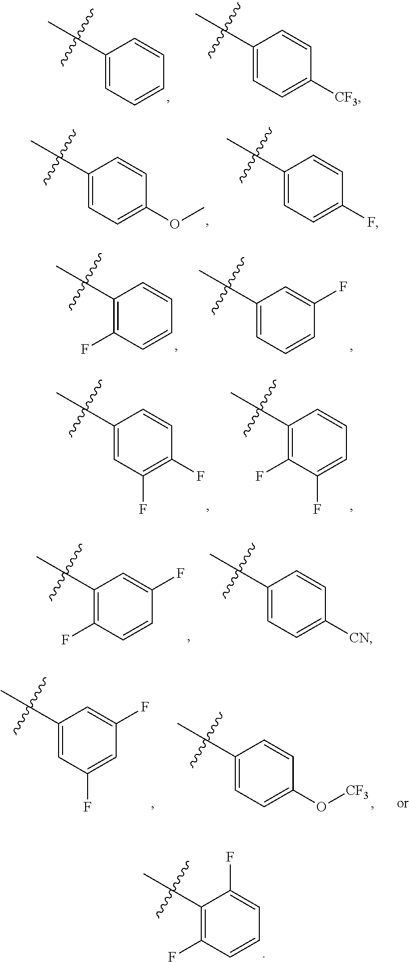

121. The pharmaceutical composition of claim 115, wherein R₁ is optionally substituted 6- to 12-membered heteroaryl.

122. The pharmaceutical composition of claim 121, wherein R₁ is

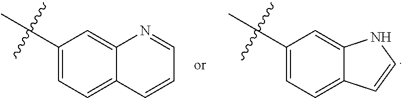 [SMILES: *c1ccc2cccnc2c1 ; *c1ccc2cc[nH]c2c1]

123. The pharmaceutical composition of claim 114, wherein the compound is a compound of formula (Ib): 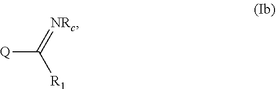
or a pharmaceutically acceptable salt or a tautomer thereof.

124. The pharmaceutical composition of claim 123, wherein R₁ is H.

125. The pharmaceutical composition of claim 123 or 124, wherein R_{c} is OR_{d}.

126. The pharmaceutical composition of claim 125, wherein R_{c} is OH.

127. The pharmaceutical composition of claim 123 or 124, wherein R_{c} is optionally substituted C₁-C₆ alkyl.

128. The pharmaceutical composition of claim 127, wherein R_{c} is methyl substituted with one or two optionally substituted C₆-C₁₆ aryl or C₁-C₁₅ heterocyclyl.

129. The pharmaceutical composition of claim 128, wherein R_{c} is

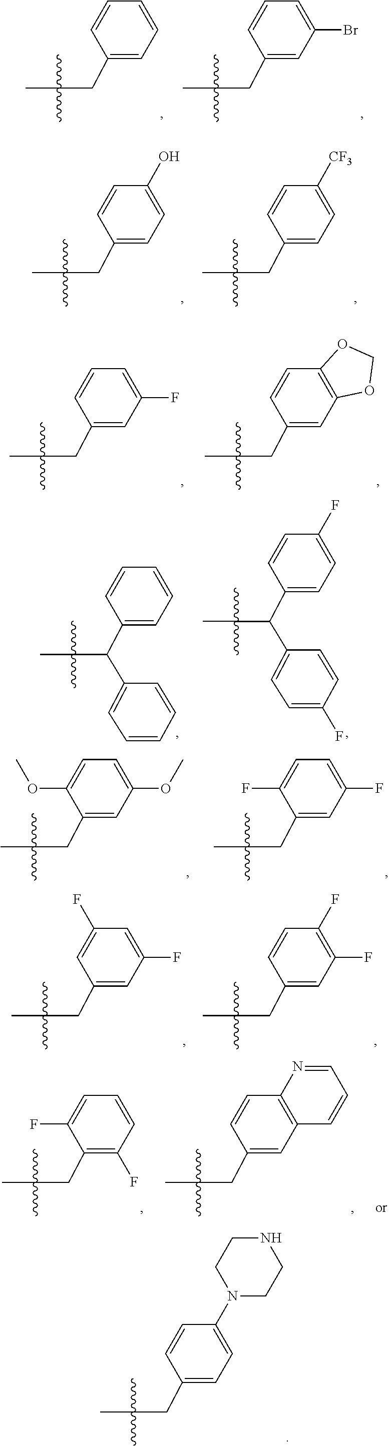

130. The pharmaceutical composition of claim 123 or 124, wherein R_{c} is optionally substituted C₆-C₁₆ aryl.

131. The pharmaceutical composition of claim 130, wherein R_{c} is

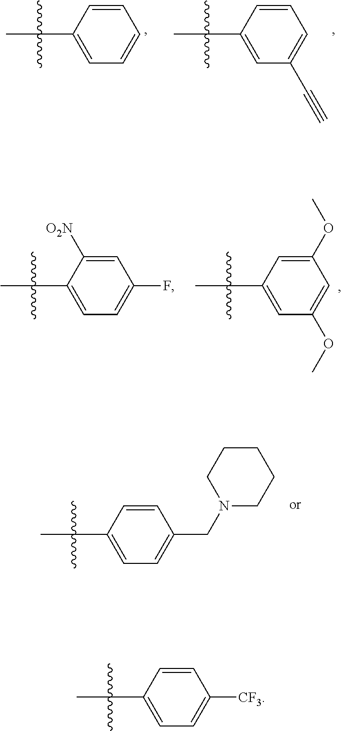

132. The pharmaceutical composition of claim 123 or 124, wherein R_{c} is optionally substituted C₁-C₁₅ heterocyclyl.

133. The pharmaceutical composition of claim 132, wherein R_{c} is

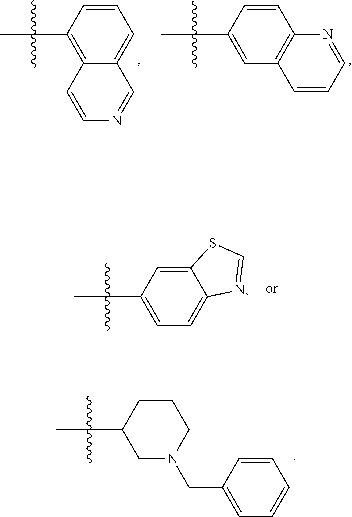 [SMILES: *c1cccc2cnccc12 ; *c1ccc2ncccc2c1 ; *c1ccc2ncsc2c1 ; "or" ; *C1CCCN(Cc2ccccc2)C1]

134. The pharmaceutical composition of claim 123 or 124, wherein R_{c} is optionally substituted C₄-C₁₃ cycloalkenyl.

135. The pharmaceutical composition of claim 134, wherein R_{c} is

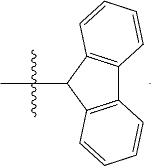 [SMILES: *C1c2ccccc2-c2ccccc21]

136. The pharmaceutical composition of claim 123 or 124, wherein R_{c} is NR_{f}R_{g}.

137. The pharmaceutical composition of claim 136, wherein R_{f} and R_{g} are independently H, optionally substituted C₁-C₆ alkyl, optionally substituted C₃-C₈ cycloalkyl, optionally substituted 6- to 10-membered heterocyclyl, or optionally substituted C₈-C₁₆ aryl.

138. The pharmaceutical composition of claim 137, wherein R_{c} is NH₂.

139. The pharmaceutical composition of claim 136, wherein R_{f} and R_{g} are independently H or optionally substituted C₈-C₁₆ aryl, wherein at least one of R_{f} and R_{g} is optionally substituted C₈-C₁₈ aryl.

140. The pharmaceutical composition of claim 139, wherein R_{c} is

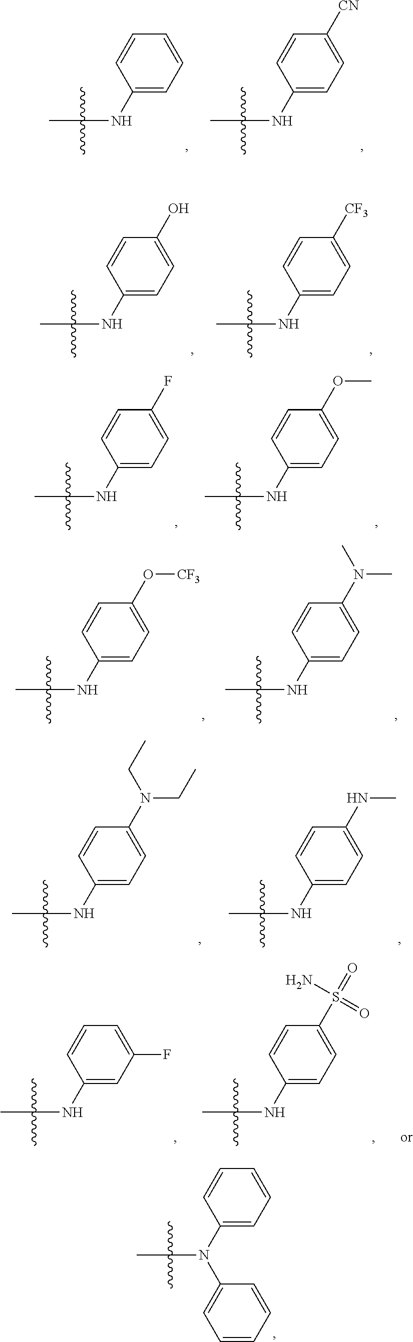

141. The pharmaceutical composition of claim 136, wherein R_{f} and R_{g} are independently H or optionally substituted C₁-C₆ alkyl, wherein at least one of R_{f} and R_{g} is optionally substituted C₁-C₆ alkyl.

142. The pharmaceutical composition of claim 141, the compound is a compound of formula (Ib-2): 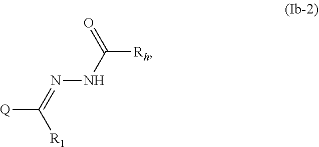
or a pharmaceutically acceptable salt thereof, wherein Rₕ is optionally substituted C₁-C₆ alkyl, optionally substituted C₃-C₈ cycloalkyl, optionally substituted C₆-C₁₆ aryl, or optionally substituted C₁-C₁₅ heterocyclyl.

143. The pharmaceutical composition of claim 142, wherein Rₕ is optionally substituted C₁-C₆ alkyl.

144. The pharmaceutical composition of claim 143, wherein Rₕ is CH₂N(CH₃)2.

145. The pharmaceutical composition of claim 142, wherein Rₕ is optionally substituted C₃-C₈ cycloalkyl.

146. The pharmaceutical composition of claim 145, wherein Rₕ is

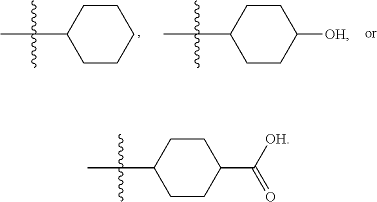 [SMILES: *C1CCCCC1 ; *C1CCC(O)CC1 ; "or" ; *C1CCC(C(=O)O)CC1]

147. The pharmaceutical composition of claim 142, wherein Rₕ is optionally substituted C₆-C₁₄ aryl.

148. The pharmaceutical composition of claim 147, wherein Rₕ is

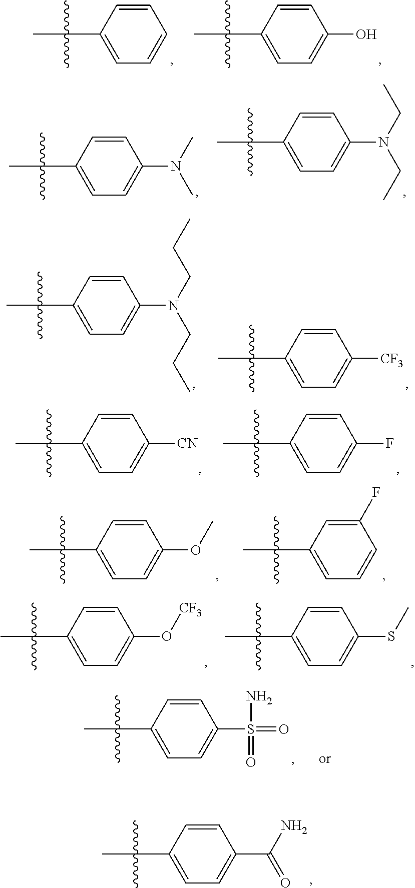

149. The pharmaceutical composition of claim 142, wherein Rₕ is optionally substituted C₁-C₁₅ heterocyclyl.

150. The pharmaceutical composition of claim 149, wherein Rₕ is

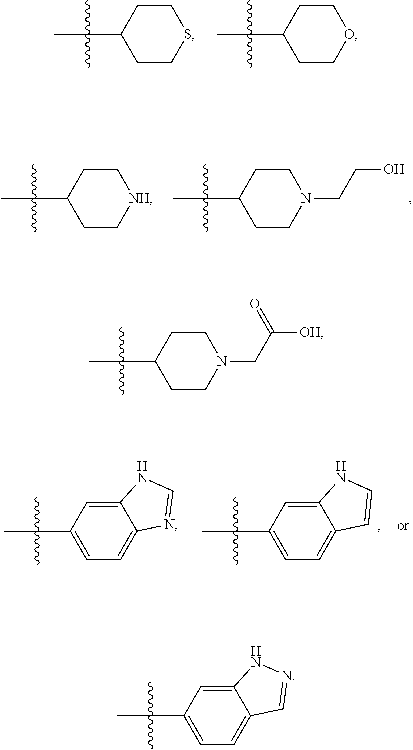

151. The pharmaceutical composition of claim 136, wherein R_{f} and R_{g} are independently H or optionally substituted C₃-C₈ cycloalkyl, wherein at least one of R_{f} and R_{g} is optionally substituted C₃-C₈ cycloalkyl.

152. The pharmaceutical composition of claim 151, wherein R_{c} is

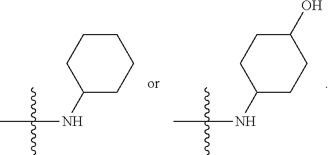 [SMILES: *NC1CCCCC1 ; *NC1CCC(O)CC1]

153. The pharmaceutical composition of claim 136, wherein R_{f} and R_{g} are independently H or optionally substituted C₁-C₁₅ heterocyclyl, wherein at least one of R_{f} and R_{g} is optionally substituted C₁-C₁₅ heterocyclyl.

154. The pharmaceutical composition of claim 153, wherein R_{c} is

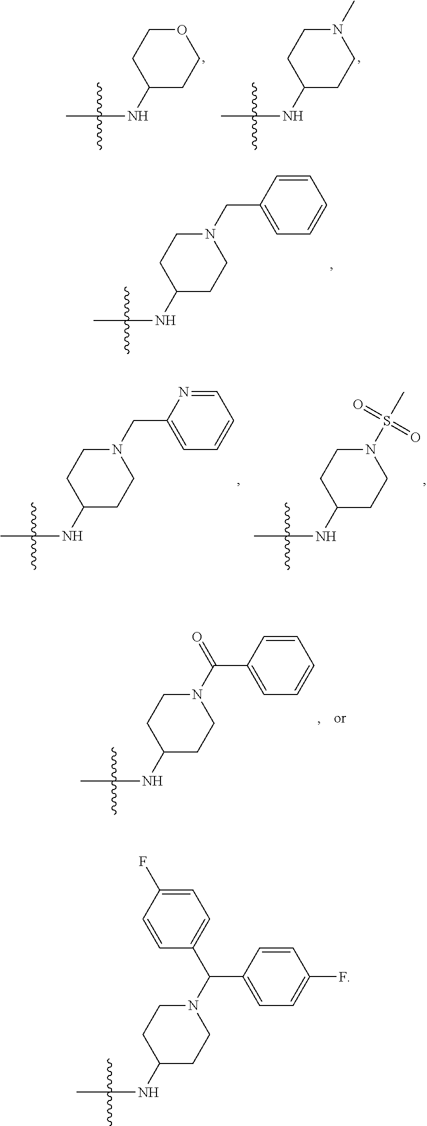

155. The pharmaceutical composition of claim 136, wherein R_{f} and R_{g}, together with the nitrogen atom to which they are attached, forms an optionally substituted 6- to 10-membered heterocyclyl.

156. The pharmaceutical composition of claim 155, wherein R_{c} is

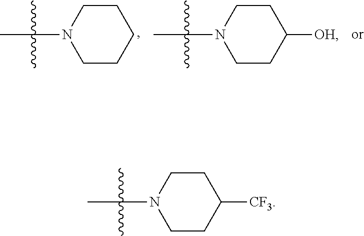 [SMILES: *N1CCCCC1 ; *N1CCC(O)CC1 ; *N1CCC(C(F)(F)F)CC1]

157. The pharmaceutical composition of claim 123 or 124, wherein R_{c} is N═C(R₁′)Q′.

158. The pharmaceutical composition of claim 157, wherein R₁′ is H.

159. The pharmaceutical composition of claim 157 or 158, wherein Q′ and Q are identical.

160. The pharmaceutical composition of claim 114, wherein [symbol] is a single bond, and R₁ and Z, together with the carbon atom to which they are attached, form an optionally substituted oxazolidinyl or optionally substituted thiazolidinyl.

161. The pharmaceutical composition of claim 160, wherein R₁ and Z, together with the carbon atom to which they are attached, form an optionally substituted thiazolidinyl.

162. The pharmaceutical composition of claim 161, wherein R₁ and Z, together with the carbon atom to which they are attached, form

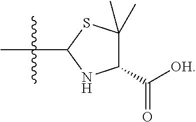 [SMILES: *C1N[C@@H](C(=O)O)C(C)(C)S1]

163. The pharmaceutical composition of any one of claims 114 to 162, wherein Q is 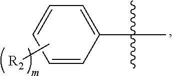
wherein each R₂ is independently halo or NRₐR_{b}, wherein Rₐ and R_{b} are independently H; optionally substituted C₁-C₆ alkyl; optionally substituted C₆-C₁₆ aryl; or SO₂Rᵢ, wherein Rᵢ is H or C₁-C₆ alkyl; or Rₐ and R_{b}, together with the nitrogen atom to which they are attached, forms an optionally substituted 5- to 10-membered heterocyclyl; and m is 0 to 5.

164. The pharmaceutical composition of claim 163, wherein m is 0.

165. The pharmaceutical composition of claim 163, wherein m is 1.

166. The pharmaceutical composition of claim 165, wherein Q is

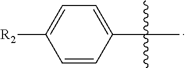

167. The pharmaceutical composition of claim 165, wherein Q is

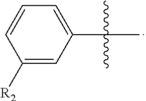

168. The pharmaceutical composition of claim 165, wherein Q is

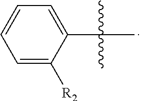

169. The pharmaceutical composition of any one of claims 165 to 168, wherein R₂ is halo.

170. The pharmaceutical composition of any one of claims 165 to 168, wherein R₂ is NRₐR_{b}.

171. The pharmaceutical composition of claim 170, wherein Rₐ and R_{b} are independently H or optionally substituted C₁-C₆ alkyl.

172. The pharmaceutical composition of claim 171, wherein R₂ is NH₂, NH(CH₃), NH(CH₂CH₃), N(CH₃)₂, N(CH₂CH₃)₂, N(CH₂CH₂CH₃)₂, or N(CH₂CH₂CH₂CH₃)₂.

173. The pharmaceutical composition of claim 172, wherein R₂ is N(CH₂CH₃)₂.

174. The pharmaceutical composition of claim 170, wherein Rₐ and R_{b}, together with the nitrogen atom to which they are attached, forms an optionally substituted 5- to 10-membered heterocyclyl.

175. The pharmaceutical composition of claim 174, wherein R₂ is

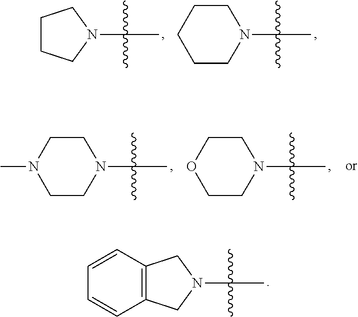

176. The pharmaceutical composition of claim 170, wherein Rₐ and R_{b} are independently H or optionally substituted C₆-C₁₆ aryl.

177. The pharmaceutical composition of claim 176, wherein R₂ is

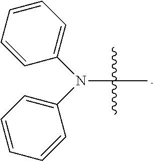 [SMILES: *N(c1ccccc1)c1ccccc1]

178. The pharmaceutical composition of claim 170, wherein R₂ is NH(SO₂CH₃).

179. The pharmaceutical composition of claim 163 wherein m is 2.

180. The pharmaceutical composition of claim 179, wherein Q is

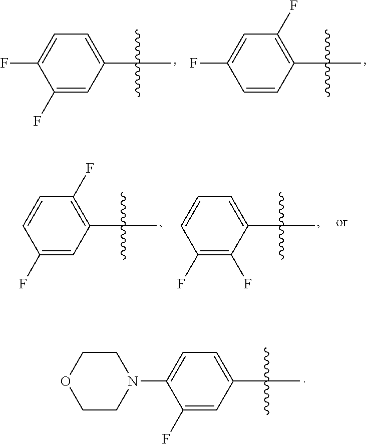 [SMILES: *c1ccc(F)c(F)c1 ; *c1ccc(F)cc1F ; *c1cc(F)ccc1F ; *c1cccc(F)c1F ; *c1ccc(N2CCOCC2)c(F)c1]

181. The pharmaceutical composition of any one of claims 114 to 162, wherein Q is optionally substituted 6- to 10-membered heterocyclyl.

182. The pharmaceutical composition of claim 181, wherein Q is

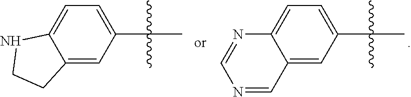 [SMILES: *c1ccc2c(c1)CCN2 ; *c1ccc2ncncc2c1]

183. The pharmaceutical composition of claim 114, wherein the compound is: 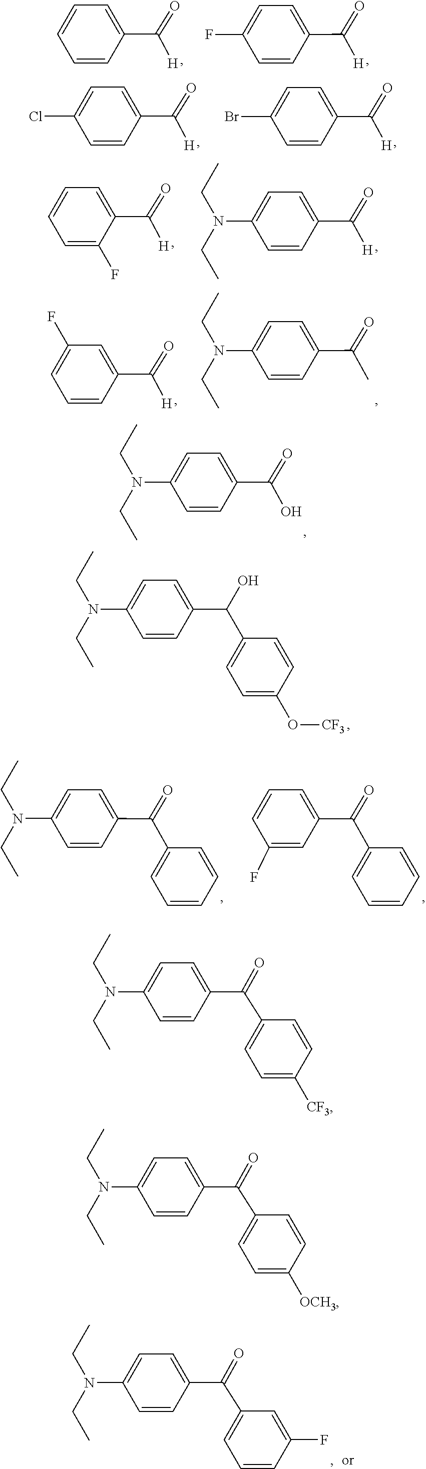 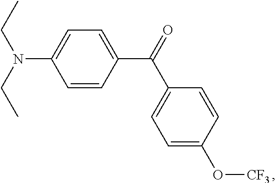 [SMILES: CCN(CC)c1ccc(C(=O)c2ccc(OC(F)(F)F)cc2)cc1]
or a pharmaceutically acceptable salt thereof.

184. The pharmaceutical composition of claim 114, wherein the compound is: 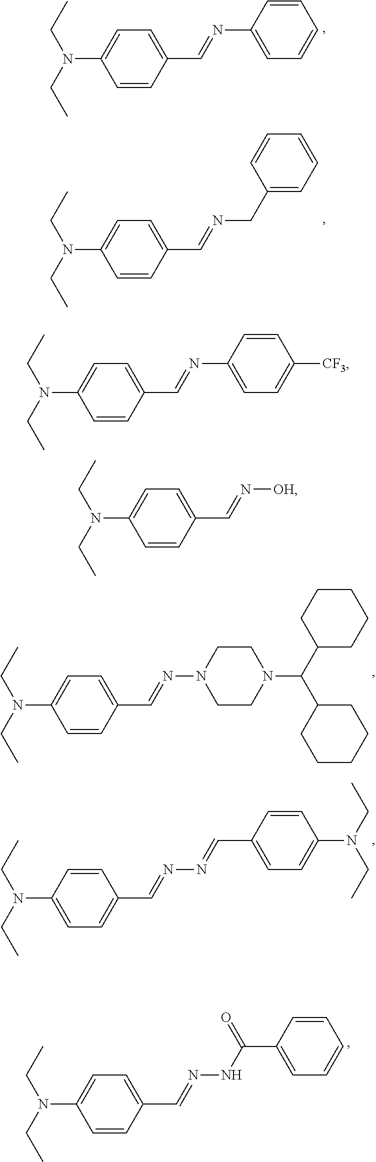 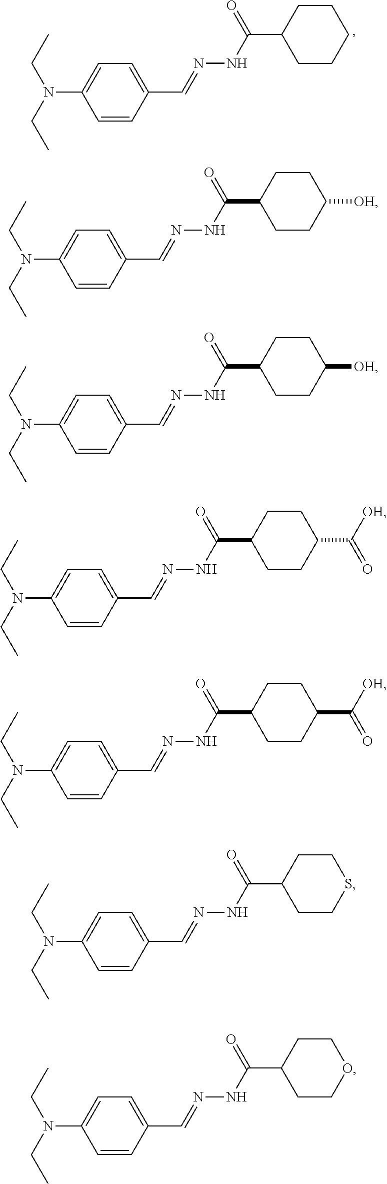 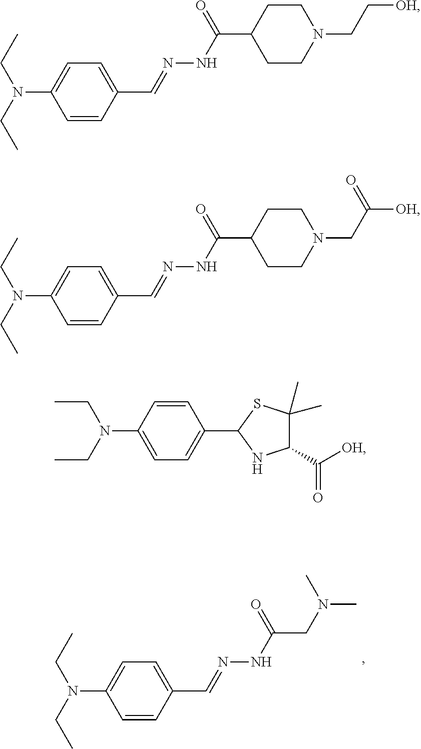 [SMILES: CCN(CC)c1ccc(/C=N/NC(=O)C2CCN(CCO)CC2)cc1 ; CCN(CC)c1ccc(/C=N/NC(=O)C2CCN(CC(=O)O)CC2)cc1 ; CCN(CC)c1ccc(C2N[C@@H](C(=O)O)C(C)(C)S2)cc1 ; CCN(CC)c1ccc(/C=N/NC(=O)CN(C)C)cc1] 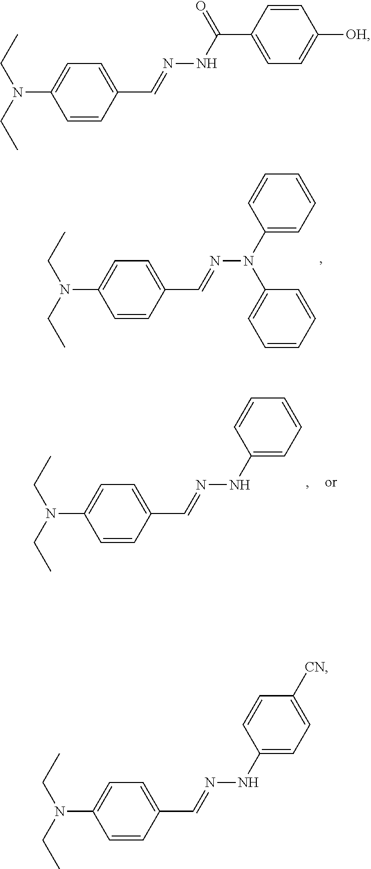 [SMILES: CCN(CC)c1ccc(/C=N/NC(=O)c2ccc(O)cc2)cc1 ; CCN(CC)c1ccc(/C=N/N(c2ccccc2)c2ccccc2)cc1 ; CCN(CC)c1ccc(/C=N/Nc2ccccc2)cc1 ; CCN(CC)c1ccc(/C=N/Nc2ccc(C#N)cc2)cc1]
or a pharmaceutically acceptable salt thereof.

185. The pharmaceutical composition of any one of claims 114 to 184 for use in the treatment of a disease or injury in a subject.

186. The pharmaceutical composition of claim 185, wherein the disease or injury is stroke; congenital hypogonadotropic hypogonadism; cerebral hemorrhage; traumatic brain injury (TBI); spinal cord injury (SCI); peripheral vascular disease (PVD); wounds; bone or cartilage injury; hearing loss; depression; anxiety; post-traumatic stress disorder (PTSD); substance abuse; peripheral nerve injury; hematopoietic disorders; amyotrophic lateral sclerosis (ALS); Alzheimer's disease; Parkinson's disease; heart disease; non-arteritic ischemic optic neuropathy (NAION); retinal artery occlusion; bronchopulmonary dysplasia, muscular dystrophy, anosmia, aging, memory disturbance, or viral infection.

187. The pharmaceutical composition of claim 186, wherein the disease or injury is stroke, provided that:
when Q is optionally substituted C₆-C₁₀ aryl, R₁ is H, Z is NR_{c}, and R_{c} is NRR_{g}, R_{f} and R_{g}, together with the nitrogen atom to which they are attached, do not form optionally substituted piperazinyl;
when Z is NR_{c}, and R_{c} is NR_{f}R_{g}, one of R_{f} and R_{g} is H, and the other of R_{f} and R_{g} is C₁-C₆ alkyl substituted with one oxo, R_{g} is not further substituted with unsaturated heterocyclyl; piperazinyl; aryl; oxo;
OR^{k}, wherein Rₖ is aryl or heterocyclyl; or NHR_{I}, wherein Rᵢ is aryl, cycloalkyl, or alkyl substituted with oxo; and
when Q is optionally substituted Ce-C₁₀ aryl and Z is O, R₁ not C₁-C₆ alkyl substituted with NHRₘ, wherein Rₘ is aryl.

188. The pharmaceutical composition of claim 187, wherein the stroke is acute stroke.

189. The pharmaceutical composition of claim 187, wherein the stroke is in a recovery phase.

190. The pharmaceutical composition of claim 186, wherein the disease or injury is congenital hypogonadotropic hypogonadism.

191. The pharmaceutical composition of claim 190, wherein the congenital hypogonadotropic hypogonadism is Kallmann Syndrome.

192. The pharmaceutical composition of claim 186, wherein the disease or injury is viral infection.

193. The pharmaceutical composition of any one of claims 114 to 184 for use in increasing spermatogenesis in a subject.

194. The method of claim 22, wherein the compound is a compound of formula (Ib-1): 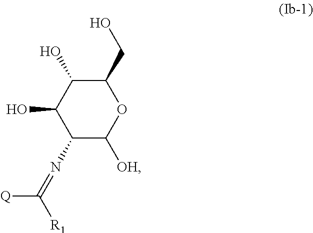
or a pharmaceutically acceptable salt or a tautomer thereof.

195. The compound of claim 81, wherein the compound is a compound of formula (Ib′-1): 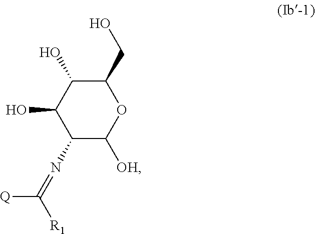
or a pharmaceutically acceptable salt or a tautomer thereof.

196. The pharmaceutical composition of claim 127, wherein the compound is a compound of formula(Ib-1): 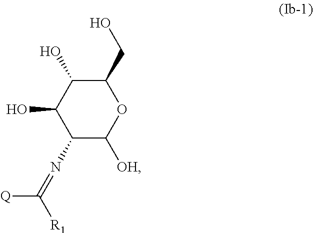
or a pharmaceutically acceptable salt or a tautomer thereof.
